(19) United States
(12) Patent Application Publication
(10) Pub. No.: US 2009/0248004 A1
(43) Pub. Date: Oct. 1, 2009

(54) SYSTEMS AND METHODS FOR TREATMENT OF SOFT TISSUE

(75) Inventors: Gregory B. Altshuler, Lincoln, MA (US); Steve Armstrong, Danvers, MA (US); Andrei V. Erofeev, North Andover, MA (US); Christopher Gaal, Mansfield, MA (US); Thomas McNall, Wakefield, MA (US); Paul Wiener, Ridgewood, NJ (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 12/396,239

(22) Filed: Mar. 2, 2009

Related U.S. Application Data
(60) Provisional application No. 61/067,712, filed on Feb. 28, 2008.
(60) Provisional application No. 61/072,939, filed on Apr. 2, 2008.

(51) Int. Cl. A61B 18/04

(52) U.S. Cl. 606/33

(57) ABSTRACT

Methods and systems for treatment of tissue (e.g., the dermal-hypodermal region) with one or more wavelength are disclosed. Electromagnetic radiation devices and methods for lypolysis and/or coagulation of blood and/or treatment of water and/or treatment of skin are disclosed.

### RELATED APPLICATIONS

This application claims the benefit of and priority to and is the non provisional application of U.S. Ser. No. 61/067,712 filed Feb. 28, 2008 entitled “System and Methods for treatment of soft tissue” and U.S. Ser. No. 61/072,939 filed Apr. 2, 2008 entitled “Systems and method for treatment of soft tissue.”

### TECHNICAL FIELD

Methods and apparatus for utilizing electromagnetic radiation, especially radiation with wavelengths between 300 nm and 100 μm, to treat cosmetic and health conditions are described. More particularly, methods and apparatus for treatment of soft tissue and tissue adjacent the dermal-hypodermal junction are described.

### BACKGROUND

In conventional liposuction localized fat is removed by a combination of a suction device, a cannula and a forceful longitudinal motion. In conventional liposuction an incision is made, the cannula tip is passed beneath the skin surface into a region of fat, the suction device is activated and tissue in the region of fat is drawn into the lumen of the cannula. The physician's forceful longitudinal motion removes the tissue in the region of that fat by combining suction and a ripping action. Liposuction is traumatic and results in trauma to the tissue in the region of the fat, blood loss, swelling, bruising, and postoperative pain. The required forceful longitudinal motion places physical demands on the physician exerting the motion to accomplish the procedure. Conventional liposuction is imprecise and corrective secondary procedures are sometimes required to provide acceptable results (e.g., to smooth out “lumpy” or “dented” surfaces).

Conventional liposuction does not treat the skin adjacent the region of fat tissue. Certain treatment areas (e.g., under the arms) and some patient's skin (e.g., the skin of older patient's) do not readily tighten around a patient's post liposuction shape. Conventional liposuction is an effective procedure for body contouring. Despite the drawbacks including, lack of precision, the physical trauma to the patient's body, and the potential for loose skin remaining post treatment, conventional liposuction is one of the most popular cosmetic surgery procedures.

Liposuction methods and devices that increase precision and reduce patient trauma could improve the outcome and reduce downtime for thousands of patients who undergo this popular cosmetic surgery procedure each year.

### SUMMARY OF THE INVENTION

In one aspect, a method for treatment of tissue with a photocosmetic device includes inserting a waveguide into a soft tissue adjacent the dermal-hypodermal junction and irradiating the soft tissue with at least one wavelength range. The waveguide is substantially rod shaped and delivers a power density ranging from about 25 W/cm² to about 1 MW/cm². In one embodiment, the waveguide has a Young's modulus that ranges from about 20 GPa to about 300 GPa, from about 65 GPa to about 150 GPa, or about 71 GPa.

In another aspect, a method for treatment of tissue with a photocosmetic device includes: inserting a treatment device into a soft tissue adjacent the dermal-hypodermal junction, irradiating the soft tissue with a first wavelength range, and irradiating the soft tissue with a second wavelength range. In one embodiment, the first wavelength range is generated prior to the second wavelength range.

Optionally, at least one of the first wavelength and the second wavelength is an optical radiation wavelength. In one embodiment, the first wavelength range is suitable for liquefying adipose tissue and the second wavelength range is suitable for treating dermis tissue. In an embodiment where the first wavelength range is suitable for liquefying adipose tissue the method also includes suctioning liquefied adipose tissue with an aspiration device.

Optionally, the second wavelength range is suitable for coagulating dermis tissue and/or for heating dermis tissue. In another embodiment, a least one of the first wavelength range and the second wavelength range tightens skin tissue. Skin tissue may be tightened by, for example, coagulating dermis tissue. The first wavelength range can be from about 910 nm to about 930 nm and the second wavelength range can be from about 970 nm to about 990 nm, for example. Other suitable wavelength ranges include 915 nm, 970 nm, 1060 nm, 1208 nm, 1440 nm, 1715 nm, and 1890 nm.

The method of treatment of tissue with the photocosmetic device can also include a third wavelength range suitable for coagulation of blood vessels. The third wavelength range can be from about 400 nm to about 700 nm. The third wavelength range includes 630 nm, for example. In one embodiment, the method of treatment of tissue employs a third wavelength range in the visible light range. The wavelength range in the visible light range can be employed as an aiming beam to assist the practitioner conducting the method of treatment.

The method of treatment of tissue with the photocosmetic device can also include measuring infrared radiation at or near an end of the waveguide or measuring infrared radiation at or near the soft tissue. Infrared radiation may be measured in single band of wavelengths or in multiple bands of wavelengths. The method of treatment of tissue can also include controlling irradiation as a function of the measured infrared radiation. The temperature of a portion of the thermal device, the soft tissue, or a combination of the portion of the thermal device and the soft tissue may be estimated using the measured infrared radiation. In one embodiment, irradiation is controlled as a function of the estimated temperature. In another embodiment, irradiation is controlled as a function of the derivative of, the integral of, or the power spectrum of the thermal radiation detected or any combination thereof. In another embodiment, irradiation is controlled as a function of specific frequency components or one or more range of specific frequency components of the power spectrum of the thermal radiation detected. Finally, the irradiation can be controlled as a function of power. In another embodiment, irradiation is controlled as a function of the intensity and/or power of infrared radiation.

The method can also include: moving a portion of the treatment device within the soft tissue and/or translating a portion of the treatment device within the soft tissue.

A portion of the treatment device adjacent the soft tissue can deliver a power density ranging from about 25 W/cm² to about 1 MW/cm². In another embodiment, a portion of the treatment device has a diameter ranging from about 200 μm to about 2 mm and a length that ranges from about 0.5 inch to about 12 inches and the ratio of the length to the diameter ranges from about 3 to about 150, or from about 50 to about 100.

In another aspect, a method for treatment of tissue with a photocosmetic device includes inserting a waveguide into a soft tissue adjacent the dermal-hypodermal junction and irradiating the soft tissue with at least one wavelength range. The waveguide is substantially rod shaped and has a Young's modulus of at least 50 GPa. In one embodiment, the waveguide has a Young's modulus that ranges from about 20 GPa to about 300 GPa, from about 65 GPa to about 150 GPa, or about 71 GPa.

In one embodiment, the waveguide is capable of emitting multiple wavelength ranges. In another embodiment, a first wavelength range is suitable for liquefying adipose tissue and a second wavelength range is suitable for treating dermis tissue. Optionally, a first optical radiation wavelength is generated prior to a second optical radiation wavelength.

In one embodiment, a method for treatment of tissue with a photocosmetic device includes inserting a waveguide into a soft tissue adjacent the dermal-hypodermal junction, irradiating the soft tissue with at least one wavelength range, and measuring infrared radiation at or near an end of the waveguide or measuring infrared radiation at or near the soft tissue. The method for treatment of tissue can also include controlling irradiation as a function of the measured infrared radiation. Optionally, the method for treatment of tissue includes using the measured infrared radiation to estimate the temperature of the end, the soft tissue, or a combination of the end and the soft tissue. In one embodiment, irradiation is controlled as a function of the estimated temperature.

The method can include moving the waveguide within the soft tissue or translating the waveguide within the region of soft tissue. The portion of the waveguide adjacent the soft tissue can deliver a power density ranging from about 25 W/cm² to about 1 MW/cm². In one embodiment, the waveguide has a diameter ranging from about 200 μm to about 2 mm and a length that ranges from about 0.5 inch to about 12 inches and the ratio of the length to the diameter ranges from about 3 to about 150, or from about 50 to about 100. In one embodiment, the end of the waveguide, e.g., the end of the waveguide that contacts the soft tissue, is configured to avoid degradation. For example, the end of the waveguide that contacts the soft tissue can be coated with a protective coating or a cap that avoids degradation.

In another embodiment, the method for treatment of tissue with a photocosmetic device includes suctioning at least a portion of the liquefied adipose tissue with an aspiration device. The liquefied adipose tissue can be suctioned simultaneously with or subsequent to irradiation of the soft tissue.

In another aspect, the method for treatment of tissue with a photocosmetic device includes inserting a waveguide into a soft tissue adjacent the dermal-hypodermal junction and irradiating the soft tissue with at least one wavelength range. The waveguide is substantially rod shaped, has a diameter ranging from about 200 μm to about 2 mm and a length that ranges from about 0.5 inch to about 12 inches and the ratio of the length to the diameter ranges from about 3 to about 150, or from about 50 to about 100.

In another aspect, a method for treatment of soft tissue with a photocosmetic device includes inserting a treatment device into a soft tissue adjacent the dermal-hypodermal junction, irradiating dermal tissue, measuring the temperature of an external surface of the dermal tissue and controlling irradiation energy as a function of the temperature. In one embodiment, irradiating the dermal tissue coagulates dermis tissue and results in a tightened appearance on the external surface of the skin. The method can also include irradiating adipose tissue. Optionally, the method further includes removing all or a portion of the liquefied adipose tissue. In one embodiment, liquefied adipose tissue is suctioned via an aspiration device.

In another embodiment, the treatment device is capable of emitting a first wavelength range and a second wavelength range. The soft tissue adjacent the dermal-hypodermal junction is irradiated with a first wavelength range and a second wavelength range. In another embodiment of the method, the treatment device is moved to irradiate multiple regions of tissue. The treatment device may be rotated to irradiate multiple regions of tissue. The method may also include providing a wavelength range visible through the external surface of the soft tissue (e.g., an aiming beam). Irradiating dermal tissue can include providing pulsed radiation or providing a preselected quantity of energy.

For example, in one embodiment, a portion of the treatment device is inserted adjacent the dermal-hypodermal junction, soft tissue is irradiated with a first wavelength range that includes the wavelength 920 nm. At least a portion of the fat in the soft tissue is liquefied upon exposure of the first wavelength range. The treatment device is rotated so that an aiming beam wavelength indicates that the device is directed toward the dermis. Then the dermis tissue is irradiated with a second wavelength range that includes the wavelength 980. The second wavelength range is applied for a length of time and at a power level that coagulates the dermal tissue. Optionally, at least a portion of the liquefied adipose tissue is removed by palpating and/or suctioning the liquefied fat from the treatment area such that it is removed from the subject's body.

In another embodiment, a method for treatment of soft tissue with a photocosmetic device includes placing a planned treatment pattern on the external surface of the dermal tissue. Optionally, irradiation of the dermal tissue may be guided by the planned treatment pattern. The treatment device may be actuated upon detection of the planned treatment pattern. In one embodiment, complementary magnets on the treatment device and on the planned treatment pattern enable detection of the planned treatment pattern by the treatment device.

The method for treatment of soft tissue with a photocosmetic device can also include sensing the soft tissue temperature, imaging the soft tissue thermal profile, sensing dermal tissue coagulation, and/or indicating dermal tissue coagulation.

In another aspect, a method for treatment of soft tissue with a photocosmetic device includes inserting a treatment device into a soft tissue adjacent the dermal-hypodermal junction, positioning the treatment device in a region of a fibrous strand partitioning the hypodermis and irradiating at least a portion of the fibrous strand. In one embodiment, the soft tissue includes cellulite. In one embodiment, the method further includes measuring the temperature of the region and controlling irradiation energy as a function of the temperature. In another embodiment, the method further includes irradiating dermal tissue. Another embodiment of the method includes first irradiating at least a portion of a fibrous strand and then rotating the treatment device and irradiating dermal tissue. In still another embodiment of the method, the temperature of an external surface of the dermal tissue is measured and irradiation energy is controlled as a function of the measured dermal tissue temperature. The method can include determining a treatment plan by cellulite location. In another embodiment, the method includes moving the treatment device to another region of a fibrous strand partitioning the hypodermis, in this way various regions of cellulite can be treated. The method can also include irradiating the soft tissue employing a wavelength range selected to coagulate blood.

In another aspect, a method for treatment of soft tissue with a photocosmetic device includes inserting a treatment device comprising an external waveguide into a soft tissue, irradiating the soft tissue with a wavelength range suitable for liquefying adipose tissue, and evacuating at least a portion of a liquefied adipose tissue through an aperture in the treatment device. Another embodiment of the method includes irradiating the soft tissue with a wavelength range suitable for treating dermis tissue. In another embodiment, a suction device is employed to evacuate at least a portion of the liquefied adipose tissue.

In another aspect, a device for the treatment of tissue adjacent the dermal-hypodermal junction includes a substantially rod shaped waveguide having a distal end configured to emit electromagnetic radiation and a proximal end configured to receive electromagnetic radiation and an interface for connecting the waveguide to a source of electromagnetic radiation. The device can include a support adjacent at least a portion of the waveguide.

The support can be, for example, a drawn wire. The support can include one or more of: a ceramic, a metal, a polymer, and a copolymer, for example. The support can have uniform thickness, non uniform thickness, a flat shape, or a concave shape, for example. The support can be adjacent the exterior surface of the waveguide. In one embodiment, the waveguide has a longitudinal axis and the support extends along at least a portion of the longitudinal axis.

In one embodiment, an adhesive bonds the support to at least a portion of the waveguide. The adhesive can cure upon exposure to ultraviolet light. Optionally, an adhesive bond fails upon exposure to sterilization conditions.

In another embodiment, the support has a Young's modulus greater than the Young's modulus of the waveguide. For example, the support has a Young's modulus that ranges from about 10 GPa to about 500 GPa, or from about 190 GPa to about 210 GPa. At least a portion of the support, e.g., the end of the support closest to the waveguide tip, may be tapered. In one embodiment, the Young's modulus of the composite formed by the waveguide and the support ranges from about 65 GPa to about 210 GPa, or about 150 GPa, for example.

In one embodiment, the distal end of the waveguide is configured to emit multiple wavelengths. For example, the distal end is configured to emit a first optical radiation wavelength range suitable for liquefying adipose tissue and a second optical radiation wavelength range suitable for treating dermis tissue.

In one embodiment, the waveguide has a power density ranging from about 25 W/cm² to about 1 MW/cm². In another embodiment, the waveguide has a diameter ranging from about 200 μm to about 2 mm and a length that ranges from about 0.5 inch to about 12 inches and the ratio of the length to the diameter ranges from about 3 to about 150, or from about 50 to about 100.

In one embodiment, the waveguide has a diameter that ranges from about 0.800 mm to about 2 mm. In one embodiment, the waveguide has a Young's modulus that ranges from about 20 GPa to about 300 GPa, from about 65 GPa to about 150 GPa, or about 71 GPa. The waveguide is substantially rod shaped and has a Young's modulus of at least 50 GPa.

The rod shaped waveguide has a distal end. The waveguide tip can be at, for example, the distal end. In one embodiment, a portion of the distal end is shaped. For example, in one embodiment a portion of the waveguide distal end is tapered. In another embodiment, a portion of the waveguide distal end is bullet shaped.

The waveguide can be fabricated from materials including quartz, sapphire, glass, or any combination of quartz, sapphire, and glass. In addition, the waveguide can be fabricated from any transparent dielectric material or any optical dielectric material. In one embodiment, the waveguide has a core fabricated from quartz, sapphire, glass, or a combination and the core is surrounded by a sheath or a buffer. In one embodiment, a sheath is sized to surround at least a portion of the waveguide and/or at least a portion of the waveguide core. Cladding can be disposed on the exterior surface of the core. The sheath can be referred to as a buffer that can be made from any of a variety of materials such as a polyamide, nylon, acrylic, a metalized coating (e.g., Copper, Gold, Aluminum deposited by for example vapor deposition), fluoropolymer (e.g., Teflon® or Tefzel®). In one embodiment, the sheath includes at least one of sapphire, glass, or quartz. The buffer can be a coating bonded directly to the core. Alternatively, the buffer is a cover that is adjacent the core. A waveguide can include a core, cladding, a buffer and/or a sheath that acts as a jacket separable from the remaining components of the waveguide (e.g., one waveguide can feature a core, cladding, and a buffer another waveguide can have a core, a buffer, and a sheath that acts as a jacket). In still another embodiment, an exterior surface of the waveguide comprises a fluoropolymer. In one embodiment, the buffer provides a safety feature in the event of a waveguide failure. More specifically, the waveguide core material is quartz, sapphire, glass or combination thereof. In use if the core were to break and/or shatter the buffer and/or sheath can maintain the pieces of core material such that they do not get lost in the subject's body.

In one embodiment, a proximal end of the waveguide is disposed in one side of an adaptor and a distal end of a fiber is disposed in the other side of the adaptor such that the proximal end of the waveguide is adjacent to the distal end of a fiber disposed in the other side of the adaptor. The fiber can be, for example, an umbilical fiber. Optionally, the fiber is referred to as an optical fiber. Optionally, the proximal end of the waveguide contacts a distal end of a fiber when both are disposed into an adaptor. An adhesive may be disposed between the proximal end of the waveguide and the distal end of the fiber to form a bond there between. In one embodiment, the adaptor mechanically aligns the waveguide and the fiber. In another embodiment, the adaptor optically aligns the waveguide and the fiber. In still another embodiment, the adaptor mechanically aligns and optically aligns the waveguide and the fiber.

In one embodiment, at least a portion of the fiber within the adaptor is surrounded by a bushing or a ferule. The adaptor may also be referred to as a connector.

The adaptor may be disposed within a hand piece (e.g., within the handle of the hand piece). Alternatively, the adaptor may be external to the hand piece handle. In one embodiment, an end of the fiber couples to a base unit that is a source of electromagnetic radiation. The end of the fiber can have a first connector adapted to connect to a second connector disposed on a base unit housing a source of electromagnetic radiation. The end of the fiber can have a first connector adapted to connect to a second connector disposed on a cluster connector housing a source of optical energy and/or a source of electromagnetic radiation.

In one embodiment of the device, the interface for connecting the waveguide to a source of electromagnetic radiation is a first connector adapted to connect to a second connector disposed on a base unit housing a source of electromagnetic radiation. In one embodiment of the device, the interface for connecting the waveguide to a source of electromagnetic radiation is a first connector adapted to connect to a second connector disposed on a cluster connector and the cluster connector attaches to a base unit housing a source of electromagnetic radiation.

Optionally, the first connector includes a material that degrades when coupled multiple times. In one embodiment, the first connector includes a material that degrades when the first connector and the second connector are decoupled. The first connector can have a locking mechanism adapted to prevent waveguide replacement or adapted to prevent coupling the first connector multiple times. In one embodiment, the waveguide has a control tag (e.g., an RFID tag) adapted to limit the length of time the waveguide may be used. In another embodiment, the waveguide has a control tag adapted to control actuation of the source of electromagnetic radiation.

The device for treatment of tissue adjacent the dermal-hypodermal junction can also include a controller. The controller can be capable of measuring a property, estimating a property, calibrating the device, controlling electromagnetic radiation, or any combination thereof. More specifically, the controller can be capable of: measuring infrared radiation at or near an end of the waveguide, measuring infrared radiation at or near the region of soft tissue, estimating the temperature at or near an end of the waveguide, estimating the temperature in the region of soft tissue, controlling optical radiation generation as a function of the measured infrared radiation, controlling optical radiation generation as a function of the estimated temperature, controlling optical radiation generation as a function of an emissive temperature at an end of the waveguide, calibrating the waveguide, controlling optical radiation generation as a function of thermal activity at the interface of an end of the waveguide and the region of soft tissue, or any combination thereof. The controller may include Si, InAs, Ge, InGaAs, two or more diodes or any combination thereof. The controller may alternatively or in addition include thermal radiation detectors (i.e., visible, near, mid or far components of infrared radiation detectors).

In another aspect, a device for treatment of soft tissue from within a region of adipose tissue includes a waveguide having a distal end configured to emit electromagnetic radiation, a support adjacent at least a portion of an exterior surface of the waveguide, and a coating. The coating surrounds at least a portion of the support and at least a portion of the waveguide and the coating binds the support to the waveguide. In one embodiment, the distal end is configured to emit multiple wavelength ranges. In another embodiment, the distal end is configured to emit a first optical radiation wavelength range suitable for liquefying adipose tissue and a second optical radiation wavelength range suitable for treating dermis tissue.

In another embodiment, the support has a Young's modulus greater than the Young's modulus of the waveguide. For example, the support has a Young's modulus that ranges from about 10 GPa to about 500 GPa, or from about 190 GPa to about 210 GPa. At least a portion of the support, e.g., the end of the support closest to the waveguide tip, may be tapered. In one embodiment, the Young's modulus of the composite formed by the waveguide and the support ranges from about 65 GPa to about 210 GPa, or about 150 GPa, for example. In one embodiment, the waveguide has a longitudinal axis and the support extends along at least a portion of the longitudinal axis.

Optionally, the device for treatment of soft tissue from within a region of adipose tissue includes a second support adjacent at least a second portion of an exterior surface of the waveguide and a third support adjacent at least a third portion of an exterior surface of the waveguide. The coating surrounds at least a portion of the support, at least a portion of the second support, at least a portion of the third support, and at least a portion of the waveguide. In one embodiment, the support, the second support, and the third support combine to have a Young's modulus greater than the Young's modulus of the waveguide. For example, the support, the second support, and the third support combine to have a Young's modulus that ranges from about 10 GPa to about 500 GPa, or from about 190 GPa to about 210 GPa. In one embodiment, the support, the second support, and the third support combine to have an effective stiffness greater than the effective stiffness of the waveguide. For example, the support, the second support, and the third support combine to have an effective stiffness that ranges from about 10 GPa to about 500 GPa, or from about 190 GPa to about 210 GPa. In one embodiment, the waveguide has a longitudinal axis and the support, the second support, and the third support extend along at least a portion of the longitudinal axis.

In one embodiment, the device includes an interface for connecting the waveguide to a source of electromagnetic radiation. The interface can be a first connector adapted to connect to a second connector disposed on a base unit housing a source of electromagnetic radiation. In one embodiment of the device, the interface for connecting the waveguide to a source of electromagnetic radiation is a first connector adapted to connect to a second connector disposed on a cluster connector and the cluster connector attaches to a base unit housing a source of electromagnetic radiation.

Optionally, the first connector includes a material that degrades when coupled multiple times. In one embodiment, the first connector includes a material that degrades when the first connector and the second connector are decoupled. The first connector can have a locking mechanism adapted to prevent waveguide replacement or adapted to prevent coupling the first connector multiple times.

The device can also include a controller. The controller can be capable of measuring a property, estimating a property, calibrating the device, controlling electromagnetic radiation, or any combination thereof.

In another aspect, a photocosmetic device for the treatment of soft tissue from within a region of adipose tissue includes a waveguide and an interface for connecting the waveguide to a source of electromagnetic radiation. The waveguide has a proximal end configured to receive electromagnetic radiation and a distal end configured to emit a first optical radiation wavelength range suitable for liquefying adipose tissue and a second optical radiation wavelength range suitable for treating dermis tissue.

In another aspect, a photocosmetic device for the treatment of soft tissue from within a region of adipose tissue includes a waveguide having a distal end configured to emit electromagnetic radiation, a tube exteriorly surrounding the waveguide, a portion of the tube extends beyond the distal end, and a cap disposed at an end of the tube, and a reflective surface is disposed on a surface of the cap adjacent the distal end and a transparent region is between the distal end and the reflective surface. In one embodiment, the tube includes materials such as quartz, sapphire, glass, metal, or hard plastic, or any combination thereof. In another embodiment, the tube is coupled to at least a portion of the waveguide by adhesive, tension fit, melting, shrink fit, or any combination thereof.

Optionally, the cap includes a heat sink. In one embodiment, at least a portion of the electromagnetic radiation emitted from the distal end of the waveguide is absorbed by the heat sink. In another embodiment, at least a portion of the cap is inserted into an open end of the tube.

The cap can have a shaped surface that is configured to contact the tissue of a subject or a patient. The cap can have a bullet shaped surface that is configured to contact a patient's tissue.

In one embodiment, at least a portion of the electromagnetic radiation emitted from the distal end of the waveguide heats the shaped surface. In another embodiment, at least a portion of the electromagnetic radiation emitted from the distal end of the waveguide refracts through the shaped surface. The shaped surface can have or be made from one or more of gold, copper, or a combination thereof.

In one embodiment, at least a portion of the electromagnetic radiation emitted from distal end of the waveguide reflects off of the reflective surface on the surface of the cap. Optionally, the reflective surface has a shape such as, for example, conical, concave, convex, angled, straight, multiple sided, single sided, curved, curved in multiple directions, or any combination thereof.

In one embodiment, the reflective surface provides a three dimensional distribution. The reflective surface can have materials including gold, copper, mirror, dielectric, multilayer dielectric or any combination thereof.

In one embodiment, the transparent region between the distal end and the reflective surface is optically transparent. The transparent region can include an adhesive gel. Alternatively, the transparent region is a void space.

In another aspect, a photocosmetic device for the treatment of soft tissue includes a tube having a width, a distal end, a proximal end, and a length between the distal end and the proximal end, the proximal end and the distal end are capped and a waveguide configured to emit electromagnetic energy is disposed along the length and at least a portion of the waveguide is inside the tube. The tube can be made of material including quartz, sapphire, glass, metal, plastic or any combination thereof. In one embodiment, a fluid (e.g., a gas such as air) is trapped inside the tube. In another embodiment, the tube is configured to move the electromagnetic energy inside the tube and along the length and the width. The waveguide can be a side firing fiber, for example. Optionally, the electromagnetic energy provides the working area.

In another aspect, a photocosmetic device for the treatment of soft tissue adjacent the dermal-hypodermal junction includes a waveguide having a proximal end configured to receive electromagnetic radiation and a distal end configured to emit electromagnetic radiation and a controller configured to receive a temperature signal. The temperature signal indicates the temperature of an external surface of dermal tissue, and wherein the controller is configured to emit electromagnetic radiation as a function of the temperature signal. In one embodiment, the waveguide is configured to emit a first wavelength range and a second wavelength range. In another embodiment, the waveguide is configured to emit a wavelength range capable of treating dermis tissue. In another embodiment, the waveguide is configured to emit a wavelength range visible through the external surface of the soft tissue.

The photocosmetic device can be configured to provide pulsed radiation. Alternatively or in addition, the photocosmetic device is configured to provide a preselected quantity of energy. In some embodiments, the photocosmetic device is configured to emit radiation according to a planned treatment pattern. The device can be configured to detect a treatment pattern. In one embodiment, the controller is configured to emit electromagnetic radiation as a function of a treatment pattern (e.g., a detected treatment pattern). In another embodiment, the device actuates waveguide emission as a function of a detected treatment pattern.

The photocosmetic device can include a thermal sensor for sensing the soft tissue temperature, a camera for imaging a thermal profile, and/or a sensor for sensing tissue coagulation and an indicator for indicating dermal tissue coagulation.

In another aspect, a photocosmetic device for the treatment of soft tissue includes a tube having a distal end, a proximal end, and a length between the distal end and the proximal end, wherein the distal end is closed and an aperture is disposed in the tube. The aperture defines an edge. A waveguide is disposed on an external surface of the tube along at least a portion of the length, wherein a portion of the waveguide is adjacent a portion of the edge and wherein the waveguide is configured to emit electromagnetic radiation suitable for liquefying adipose tissue.

In one embodiment, the at least a portion of the aperture is at an angle relative to a longitudinal axis of the tube. In another embodiment, at least a portion of the tube comprises an indentation and at least a portion of the waveguide is disposed in the indentation. The tube may be sized to evacuate liquefied adipose tissue. The device can also include a vacuum configured to evacuate at least a portion of the tube. Optionally, the distal end is closed by a cap.

In another aspect, a photocosmetic device for the treatment of soft tissue includes a tube having a distal end, a proximal end, and a length between the distal end and the proximal end, the distal end is closed. An aperture is disposed along the length of the tube. A waveguide is disposed along at least a portion of the length and at least a portion of the waveguide is located substantially opposite the aperture. An end portion of the waveguide is configured to emit electromagnetic radiation and the at least a portion of the waveguide is configured to emit electromagnetic radiation through the aperture. In one embodiment, the tube is sized to evacuate liquefied adipose tissue. In another embodiment, a vacuum is configured to evacuate at least a portion of the tube.

In one embodiment, at least a portion of the waveguide is configured to emit a wavelength range suitable for liquefying adipose tissue. The end portion of the waveguide may be configured to emit a wavelength range suitable for dermis coagulation or for blood coagulation. In one embodiment, the waveguide has a coating and the at least a portion of the waveguide is etched.

In some embodiments, the closed distal end is a heat sink. In other embodiments, the closed distal end is insulated. Optionally, at least a portion of the electromagnetic radiation emitted from the waveguide is absorbed by the closed distal end.

In another aspect, a photocosmetic device for the treatment of soft tissue includes a tube having a distal end, a proximal end, and a length between the distal end and the proximal end. The distal end is chamfered and a waveguide is disposed in a portion of the tube. The waveguide is configured to emit electromagnetic radiation along an inside surface of at least a portion of the tube and the waveguide is configured to emit electromagnetic radiation that reflects off of the distal end. The photocosmetic device may also include a vacuum force configured to evacuate fluid from the distal end toward the proximal end. The distal end can have a beveled shape. The tube can be made from any of a number of materials including sapphire, quartz, glass, metal or a combination thereof. The tube may comprise a metal support member or a metal support layer, for example.

In another aspect, a photocosmetic device for the treatment of soft tissue includes a tube having a distal end, a proximal end, and a length between the distal end and the proximal end, the distal end is closed. An aperture is disposed along the length of the tube. A reflective surface is disposed on an inside surface of the tube on a side substantially opposite the aperture. A waveguide is configured to emit electromagnetic energy and at least a portion of the waveguide is disposed inside the tube. The photocosmetic device can have a vacuum force configured to evacuate fluid from the distal end toward the proximal end. The tube can be made from any of a number of materials including sapphire, quartz, glass, metal or a combination thereof. In one embodiment, the closed distal end is a heat sink. In another embodiment, the closed distal end is insulated. Optionally, at least a portion of the electromagnetic radiation emitted from the waveguide is absorbed by the closed distal end.

In another aspect, a method for treatment of tissue with a device includes inserting a treatment device into a soft tissue on an inside surface of a subject's body. The soft tissue is irradiated with a first wavelength range of electromagnetic radiation and the soft tissue is irradiated with a second wavelength range of electromagnetic radiation. In one embodiment, the first wavelength range is suitable for liberating lipid from adipose tissue and the second wavelength range is suitable for treating at least one of proteins, fibrotic components of adipose tissue, and skin tissue. In another embodiment, the first wavelength range is suitable for melting adipose tissue and the second wavelength range is suitable for coagulating skin tissue or ablating skin tissue.

The portion of the treatment device that is inserted into the subject's body can be the waveguide, for example, a self supporting waveguide. In another embodiment, the treatment device has a side firing waveguide. The method can also include the step of generating electromagnetic radiation. In some embodiments, the method includes initiation of increased absorption of at least one wavelength at or near an end (e.g., the proximal end) of the treatment device prior to insertion. Initiation of increased absorption can be, for example, carbonization.

The method for treatment of tissue can include measuring the infrared radiation at or near an end of the treatment device or measuring infrared radiation at or near the irradiated soft tissue and controlling irradiation as a function of the measured infrared radiation. In one embodiment, the treatment device includes a waveguide forming a tube that is adapted to evacuate at least a portion of irradiated tissue through the tube. The treatment device has an aperture adapted to evacuate at least a portion of irradiated tissue.

In a method for treatment of tissue with a device that includes inserting a treatment device into a soft tissue on an inside surface of a subject's body the soft tissue can be irradiated with a first wavelength range of electromagnetic radiation and the soft tissue can be irradiated with a second wavelength range of electromagnetic radiation. In one embodiment, the first wavelength range is suitable for melting adipose tissue and the second wavelength range is suitable for ablating one or more holes in skin tissue. At least a portion of the melted adipose tissue can be removed through the one or more holes in skin tissue. Alternatively or in addition, one or more of the holes formed in the skin tissue can be filled with adipose tissue. The adipose tissue that fills the one or more holes can be melted adipose tissue or can be non-melted (e.g., intact) adipose tissue. For example, a negative pressure such as a vacuum can be employed to pull the adipose tissue through one or more of the holes. Intact adipose tissue includes at least some stem cells, employing negative pressure to move intact adipose tissue into one or more holes in the skin tissue can deliver a subject's own stem cells into the subject's skin tissue. Optionally, some of the holes can be filled with melted adipose tissue and other of the holes can be filled with intact adipose tissue. In one embodiment, adipose tissue removal is assisted by applying suction to the one or more holes in the skin to thereby to remove the adipose tissue through the one or more holes in the skin tissue (e.g., melted adipose tissue and/or intact adipose tissue can be removed via suction).

In a method for treatment of tissue with a device that includes inserting a treatment device into a soft tissue on an inside surface of a subject's body the soft tissue can be irradiated with a first wavelength range of electromagnetic radiation and the soft tissue can be irradiated with a second wavelength range of electromagnetic radiation. The method can further include removing at least a portion of a pigmented lesion, at least a portion of a vascular lesion, at least a portion of a wrinkle, or at least a portion of a tattoo with a wavelength range of electromagnetic radiation suitable for ablating soft tissue or a wavelength range of electromagnetic radiation suitable for coagulating soft tissue. In one embodiment, the second wavelength range is suitable for coagulating skin tissue. In another embodiment, at least one of the first wavelength range and the second wavelength range tightens skin tissue. In another embodiment, at least one of the first wavelength range and the second wavelength range tightens adipose tissue. Adipose tissue may be tightened when, for example, the adipose tissue is treated with a wavelength suitable for melting and/or liberating lipids from the adipose tissue. When one does a treatment of adipose tissue to melt and/or liberate lipids from the adipose tissue proteins and/or fibrotic tissue are coagulated. The fibrotic tissue dictates, at least in part, the volume of the tissue. By coagulating fibrotic tissue you can get shrinkage of fibrotic tissue which will lead to contraction and/or retraction of adipose tissue (e.g., subcutaneous fat). There are various ways to provide an appearance of shrinking skin, for example, wavelength range(s) of electromagnetic energy can be applied to coagulate and shrink: (1) only skin, (2) only adipose tissue or (3) skin and adipose tissue together. Shrinking adipose tissue (e.g., subcutaneous fat) by coagulating proteins and/or fibrotic tissue improves the tightened appearance at least in part because when the tissue contracts, because skin is elastic, the skin follows the shrunken proteins and/or fibrotic tissue to lead to a more tightened appearance.

In a method for treatment of tissue with a device that includes inserting a treatment device into a soft tissue on an inside surface of a subject's body the soft tissue can be irradiated with a first wavelength range of electromagnetic radiation and the soft tissue can be irradiated with a second wavelength range of electromagnetic radiation. The first wavelength range may be generated prior to the second wavelength range. In addition, the method can also include a third wavelength range suitable for coagulation of blood vessels. In one embodiment, the melted adipose tissue may be suctioned with an aspiration device.

In another aspect, a method for treatment of tissue with a device includes inserting a self supporting waveguide into a soft tissue adjacent on an inside surface of a subject's body and irradiating the soft tissue with at least one wavelength range of electromagnetic radiation. The method can also include, generating electromagnetic radiation. In one embodiment, the waveguide is capable of delivering power with multiple wavelength ranges. In one embodiment, the first wavelength range is suitable for liberating lipid from adipose tissue and a second wavelength range is suitable for treating at least one of proteins, fibrotic components of adipose tissue, and skin tissue. A wavelength range suited to treating skin tissue (e.g., suited to coagulating and/or ablating skin tissue, for example, from about 970 nm to about 990 nm) may be employed to treat at least one of proteins, fibrotic components of adipose tissue. In one embodiment, a first optical radiation wavelength is generated prior to a second optical radiation wavelength. A melted adipose tissue may be suctioned with an aspiration device, for example. The method of treating tissue with a self supporting waveguide can also include moving the waveguide within the soft tissue.

In another aspect, a device for the treatment of tissue inside a subject's body includes a source of electromagnetic radiation, a self supporting waveguide having a distal end configured to deliver electromagnetic radiation and a proximal end configured to receive electromagnetic radiation, and an interface for connecting the waveguide to the source of electromagnetic radiation. At least a portion of the waveguide may have had an initiation of increased absorption of at least one wavelength (e.g., by carbonization). The device can also include a means to measure the infrared radiation at or near an end of the treatment device or a means to measure infrared radiation at or near the irradiated soft tissue and a means to control irradiation as a function of the measured infrared radiation. In one embodiment, the waveguide is adapted to side fire. In one embodiment, one portion of the waveguide is made from a first material and another portion of the waveguide is made from a second material. The first portion of the waveguide may be made from transparent material with refractive index of at least 1.51. Alternatively or in addition, the first portion of the waveguide is made from sapphire. In one embodiment, the waveguide that is adapted to side fire, substantially side fires in one type of tissue and the waveguide fires substantially along the longitudinal axis in another type of tissue.

In one embodiment, of a device for the treatment of tissue inside a subject's body that includes a source of electromagnetic radiation and a self supporting waveguide, the waveguide is hollow. Alternatively or in addition, the waveguide can be a tube. The tube of the waveguide (e.g., the waveguide tube) can include a transparent material. The waveguide tube can optionally include a suction means that pulls tissue from a distal end of the tube to a proximal end of the tube. In some embodiments, the waveguide tube leaks electromagnetic radiation along at least a portion of the inside surface of the tube.

In one embodiment of a device for the treatment of tissue inside a subject's body that includes a source of electromagnetic radiation and a self supporting waveguide a portion of the distal end is shaped. The distal end can be, for example, bullet shaped. In one embodiment, the proximal end of the waveguide is disposed in one side of an adaptor, the proximal end of the waveguide is adjacent to a distal end of a fiber disposed in the other side of the adaptor. In another embodiment, the proximal end of the waveguide is disposed in one side of an adaptor, the proximal end of the waveguide contacts a distal end of a fiber disposed in the other side of the adaptor. The adaptor can mechanically align and/or optically align the waveguide and the fiber. In one embodiment, the adaptor is disposed within a hand piece. An end of the fiber may be coupled to a base unit housing with source of electromagnetic radiation. The interface may be a first connector adapted to connect to a second connector disposed on a base unit housing a source of electromagnetic radiation. The first connector can include a locking mechanism adapted to prevent waveguide replacement. The first connector can include a locking mechanism adapted to prevent coupling the first connector multiple times. The device can also include a control tag adapted to limit the length of time the waveguide may be used. The control tag may also be adapted to control actuation of the source of electromagnetic radiation. In one embodiment, the device includes a sheath sized to surround at least a portion of the waveguide and the sheath is adapted to contain at least a portion of the waveguide when the waveguide breaks.

In some embodiments, the distal end of the waveguide is configured to deliver multiple wavelengths of electromagnetic radiation. In one embodiment, the distal end of the waveguide is configured to deliver a first optical radiation wavelength range suitable for melting, coagulating or inducing apoptosis of adipose tissue and a second optical radiation wavelength range suitable for treating skin tissue. In one embodiment, the device includes a tube adjacent the waveguide. The waveguide may be external to the tube.

In another aspect, a method for treatment of tissue with a device includes inserting a waveguide into a soft tissue adjacent the dermal-hypodermal junction on the inside surface of a subject's body and irradiating the soft tissue with a wavelength range of electromagnetic radiation suitable for ablating skin tissue, thereby forming one or more hole in the skin tissue. In one embodiment, a first end of the one or more hole is on the inside surface of the subject's body and a second end of the one or more hole is on the external surface of the subjects body. In another embodiment, a first end of the one or more hole is on the inside surface of the subject's body and a second end of the one or more hole is on the inside surface of the subject's body from about 0.1 mm to about 1 mm below the external surface of the subject's body.

The method for treatment of tissue may also include generating electromagnetic radiation. The wavelength range may be from about 1400 nm to about 11000 nm. In one embodiment, the one or more hole has the shape of a cone with the first end of the one or more hole having a larger diameter and the second end of the one or more hole having a smaller diameter.

In accordance with the method of treatment of tissue, ablating the one or more holes can: tighten skin tissue, remove at least a portion of a wrinkle in the skin tissue, remove at least a portion of a pigmented lesion in the skin tissue, remove at least a portion of a vascular lesion in the skin tissue, and/or remove at least a portion of a tattoo. In accordance with the method, irradiating the soft tissue with a second wavelength range can include a wavelength range of from about 910 nm to about 935 nm or from about 1200 nm to about 1740 nm. In one embodiment, the method can also include irradiating the soft tissue with a second wavelength range suitable for melting adipose tissue. In some embodiments, at least a portion of the melted adipose tissue may be removed through the one or more holes in the skin tissue. Optionally, suction may be applied to the one or more holes to assist melted adipose tissue removal through the one or more holes in skin tissue. The one or more holes in the skin tissue may be filled with at least a portion of the adipose tissue. The method can include controlling formation of the one or more holes in the skin tissue to provide a desired distance between adjacent holes. The method can also include controlling formation of the one or more holes in the skin tissue to provide areas of untreated skin tissue surrounding the one or more holes formed in the skin tissue.

In another aspect, a device for the treatment of tissue inside a subject's body includes a source of electromagnetic energy, a waveguide having a distal end configured to deliver electromagnetic radiation in a selective direction and a proximal end configured to receive electromagnetic radiation and an interface for connecting the waveguide to a source of electromagnetic radiation. In one embodiment, the distal end is angled thereby to enable the waveguide to deliver a substantial portion of the electromagnetic radiation at an angle of greater than about 10 degrees relative to the waveguide longitudinal axis. In another embodiment, one portion of the waveguide is made from a first material and another portion of the waveguide is made from a second material. In one embodiment, the first portion of the waveguide is made from a material with refractive index of at least 1.51. In another embodiment, the first portion of the waveguide is made from a sapphire material. In still another embodiment, the waveguide substantially side fires in one type of tissue and the waveguide fires substantially along the longitudinal axis in another type of tissue.

In another aspect, a method for treatment of tissue with a device includes inserting a waveguide into an inside surface of a subject's body, wherein the waveguide has a distal end configured to deliver electromagnetic radiation in a selective direction and irradiating a soft tissue with at least one wavelength range of electromagnetic radiation. The method can also include generating electromagnetic radiation. In one embodiment, the distal end substantially side fires in one type of tissue and the distal end fires substantially along the longitudinal axis in another type of tissue. In another embodiment, an angled distal end delivers a substantial portion of the electromagnetic radiation at an angle of greater than 10 degrees relative to the waveguide longitudinal axis. In another embodiment, an angled distal end delivers a substantial portion of the electromagnetic radiation at an angle of greater than 30 degrees relative to the waveguide longitudinal axis. In one embodiment, the at least one wavelength range is suitable for coagulating soft tissue. In another embodiment, the wavelength range releases tension in at least a portion of the soft tissue. In one embodiment, the at least one wavelength range is suitable for ablating soft tissue. In another embodiment, the wavelength range cuts the soft tissue. In one embodiment, the soft tissue is septa tissue. In another embodiment, the soft tissue is fibrous septa. In another embodiment, the soft tissue is skin tissue. In one embodiment, the method can include aligning the soft tissue with the waveguide by employing an aiming beam. In another embodiment, the method can include applying an aiming beam inside the subject's body for treatment feedback control.

In another aspect, a device for the treatment of tissue inside a subject's body includes a source of electromagnetic energy, a waveguide having a distal end configured to deliver electromagnetic radiation and a proximal end configured to receive electromagnetic radiation and a tube having a distal end and a proximal end, the tube comprising a suction means that pulls tissue from the distal end to the proximal end. In one embodiment, the waveguide forms the tube and the waveguide can be, for example, a transparent tube.

In another embodiment, the tube leaks electromagnetic radiation along at least a portion of the inside surface of the tube. In another embodiment, the waveguide is adjacent the tube. The waveguide may be external the tube. In one embodiment, waveguide is adapted to side fire. In another embodiment, the suction means is adapted to pull the tissue that has been irradiated (e.g., a substantial portion of the tissue that has been irradiated or solely the tissue that has been irradiated).

In another aspect, a method for the treatment of tissue inside a subject's body includes inserting a waveguide into an inside surface of a subject's body, the waveguide is configured to deliver electromagnetic radiation, inserting a tube into an inside surface of a subject's body, the tube has a distal end and a proximal end, irradiating a soft tissue with at least one wavelength range of electromagnetic radiation and suctioning irradiated soft tissue from the distal end to the proximal end. The method can also include generating electromagnetic radiation. The power of electromagnetic radiation may be adjusted to melt adipose tissue at a volume rate substantially the same as the rate of suctioning. In another embodiment, the suctioning means is controlled to suction the irradiated soft tissue (e.g., substantially the irradiated tissue or substantially only the irradiated tissue). In one embodiment, the power applied controls at least a portion of the suctioning means. In another embodiment, the applied speed controls at least a portion of the suctioning means. Optionally, the aspiration rate can control at least a portion of the suctioning means.

In another aspect, a device for the treatment of tissue inside a subject's body includes a waveguide having a distal end configured to deliver electromagnetic radiation and a proximal end configured to receive electromagnetic radiation and a spacer. At least a portion of the spacer is adapted to maintain a portion of the spacer and a portion of the waveguide at a substantially constant distance there between. The device can include a source of electromagnetic energy. In one embodiment, the portion of the waveguide is the distal end. In another embodiment, the at least a portion of the spacer is adapted to contact an outside surface of a subject's skin. The at least a portion of the spacer can be at least one of a roller, a skate, a ski, and a ball. The at least a portion of the spacer can be a shape suitable for movement on the external surface of the contact surface. The at least a portion of the spacer can be translucent. In one embodiment, the waveguide is adapted to deliver an aiming beam. Optionally, at least one of the waveguide and the spacer further include a magnet. In another embodiment, the waveguide distal end is adapted to side fire.

In another aspect, a method for the treatment of tissue inside a subject's body includes inserting a waveguide into an inside surface of a subject's body and contacting an external surface of the subject's body with at least a portion of a spacer, the at least a portion of the spacer adapted to maintain a portion of the spacer and a portion of the waveguide at a substantially constant distance there between. The method includes traversing a portion of the inside surface of the subject's body with the waveguide and substantially simultaneously traversing a portion of the outside surface of the subject's body with the at least a portion of the spacer. The method can also include generating electromagnetic radiation. In one embodiment, the method includes irradiating a soft tissue of the subject's body from the inside surface with a first wavelength range of electromagnetic radiation. The first wavelength range can coagulates skin tissue or can ablate skin tissue. In one embodiment, a distal end of the waveguide substantially side fires in one type of tissue and the distal end fires substantially along the longitudinal axis in another type of tissue. In another embodiment, the waveguide has a distal end configured to deliver electromagnetic radiation in a selective direction. Optionally, a soft tissue may be aligned with the waveguide via an aiming beam. In one embodiment, the waveguide melts and coagulates adipose tissue at a depth predetermined by the substantially constant distance.

In another aspect, a method includes generating electromagnetic radiation, inserting a waveguide into a region comprising adipose tissue, and applying radiation with a wavelength range of from about 910 nm to about 935 nm, for a period of time and at a power level to liberate lipid from the adipose tissue and at a temperature of the irradiated adipose tissue below the temperature at which all or a portion of the irradiated adipose tissue vaporizes. The volume percentage of liberated lipid is at least two times the volume percentage of lipid liberated by an applied wavelength range including a wavelength selective to lipid chromophores not including about 910 nm to about 935 nm applied for the period of time, at the power level and below the temperature. The wavelengths selective to lipid chromophores include from about 910 nm to about 935 nm, from about 1200 nm to about 1230 nm, from about 1700 nm to about 1740 nm or from about 2300 nm to about 2320 nm.

### DESCRIPTION OF THE FIGURES

FIG. 1 is a view of the system that delivers electromagnetic radiation; the system includes a cart, a base unit and a hand piece.

FIG. 2 is a front view of the base unit.

FIG. 3A is a view of the cluster connector that connects the hand piece to the base unit, the cluster connector includes a diode laser assembly, a cooling plate, and a fiber coupler assembly.

FIG. 3B is a schematic view of the fiber coupler assembly.

FIG. 3C is a schematic view of a diode laser assembly.

FIG. 3D is another schematic view of a diode laser assembly.

FIG. 3E is an absorption spectra of fat, dermis, vein, and arterial tissue measured in mm⁻¹ versus wavelength measured in nm.

FIG. 3F shows the ratio of temperature rise of subcutaneous fat compared to the temperature rise of dermis as a function of an applied wavelength in nm.

FIG. 3G is an electron micrograph of adipocyte and surrounding tissue.

FIG. 3H is a photomicrograph of unilocular adipose tissue (WAT) showing lipid (L) within the adipocytes (A), the cytoplasm is visible in some areas and the nucleus (N) is seen along the periphery of the adipocyte cell. Also visible is a capillary (C) and venule (V).

FIG. 3I is a representation of the architecture of subcutaneous adipose tissue.

FIG. 3J shows the absorption spectrum, measured by the absorption coefficient in m⁻¹, of human fat under physiologic conditions, of human fat mixed with 15% water, and of human fat mixed with 30% water versus the applied wavelength measured in nm. The samples mixed with water provide the conditions that exist following tumescence.

FIG. 3K shows the Absorption spectrum, measured by the absorption coefficient in m⁻¹, of dermis with 70% water and fat under physiologic conditions versus the applied wavelength measured in nm.

FIG. 3L shows in grayscale the temperature profile at the end of the 1 s pulse around the bullet shaped tip operating in mixed mode at 25 W. Also shown are the liberated lipid and coagulation zones created in the tissue.

FIG. 3M shows the volume percentage of liberated lipid volume (LLV) and the volume percentage of coagulated collagen volume (CCV) in adipose tissue in normal physiologic condition versus the applied wavelength to achieve the volume.

FIG. 3N shows a generalized plot of the volume of fat (e.g., adipose tissue) treated (e.g., melted or lipid liberated) as a function of absorption coefficient.

FIG. 4 is a side view of a hand piece having a handle with a fiber at one end that connects to a base unit, the other end of the handle has a waveguide.

FIG. 5A is a view of a waveguide tip.

FIG. 5B is a side view of a shaped waveguide tip.

FIG. 5C is a side view of a raytrace of the predicted optical energy pattern emitted from the waveguide tip shown in FIG. 5B.

FIG. 5D is a side view of another shaped waveguide tip.

FIG. 5E is a side view of a raytrace of the predicted optical energy pattern emitted from the waveguide tip shown in FIG. 5D.

FIG. 6 is a cross sectional view of the handle of the hand piece, the handle has a fiber at one end that connects to a base unit, the other end of the handle has a waveguide.

FIG. 7 is another cross sectional view of the handle of the hand piece and a cross sectional view of the handle cap that connects a waveguide to the handle.

FIG. 8A is a view of a waveguide held by a handle cap.

FIG. 8B is a view of a side firing waveguide in use inside a subject's body.

FIG. 8C is a view of the surface of a subject's body with ablative holes disposed through the surface.

FIG. 8D is a view of a side firing waveguide in use inside a subject's body to treat the dermis and epidermis.

FIG. 8E is a view of a side firing waveguide in use inside a subject's body to treat the fat tissue.

FIG. 8F is a view of a side firing waveguide applying energy to one or more fiber stents inside a subject's body to improve the appearance of cellulite.

FIG. 8G is a view of a side firing waveguide applying energy inside a subject's body in a region of fat tissue and in a region of to one or more fiber stents.

FIG. 9 is a cross sectional view of an adaptor that connects the fiber to the waveguide in the region of the hand piece (e.g., in the handle of the hand piece).

FIG. 10 is a view of a tube with at least a portion of a waveguide disposed in at least a portion of the tube and all or a portion of an interior surface of the tube is reflective.

FIG. 11 is a view of a waveguide combined with an aspiration tube, the aspiration tube can suction through an aperture in a cover that surrounds the waveguide and the aspiration tube.

FIG. 12 is a cross sectional view of the waveguide combined with an aspiration tube and surrounded by a cover shown in FIG. 11.

FIG. 13 is a cross sectional view of the handle showing the connection of the fiber to the waveguide and showing a waveguide together with an adjacent support.

FIG. 14 is another view of the cross section of the handle showing the connection of the fiber to the waveguide and showing a waveguide together with an adjacent support.

FIG. 15 is a view of the tip of the waveguide and the adjacent support, the waveguide has a core and a buffer.

FIG. 16 is a cross section of the waveguide and an adjacent support.

FIG. 17 is a cross section of the waveguide and an adjacent support.

FIG. 18 is a cross section of a waveguide including a core surrounded by a buffer, with a first support, a second support, and a third support adjacent the waveguide.

FIG. 19 is a cross section of a waveguide including a core surrounded by a buffer, with a first support, a second support, and a third support adjacent the waveguide, a coating surrounds at least a portion of: the waveguide, the first support, the second support and the third support.

FIG. 20 is a view of a waveguide having a core surrounded by a buffer, a first support, a second support, and a third support adjacent the waveguide, a coating surrounds at least a portion of: a first support, a second support, a third support and the waveguide.

FIG. 21 is a view of a waveguide, a first support, a second support, and a third support adjacent the waveguide, a coating surrounds at least a portion of: a first support, a second support, a third support and the waveguide, a cover is disposed at the distal end of the waveguide and a tip at the distal end of the waveguide exits an aperture in the cover.

FIG. 22 is a view of a waveguide, a tube surrounds the waveguide and a cap is disposed at the end of the tube.

FIG. 23 is a view of a tube with at least a portion of a waveguide disposed in at least a portion of the tube and the tube distributes electromagnetic energy to a treatment area.

FIG. 24 is a view of a tube with at least a portion of a waveguide disposed in at least a portion of the tube, at least a portion of the tube is selectively coated with insulative coating and portion of the tube are free from insulative coating and the tube distributes electromagnetic energy to a treatment area

FIG. 25 is a view of a tube with an aperture disposed in the tube and a waveguide is disposed substantially opposite the aperture.

FIG. 26 is a view of a tube, an aperture is disposed in the tube, a waveguide is disposed substantially opposite the aperture, a portion of the waveguide is configured to emit electromagnetic radiation through the aperture and a portion of the waveguide is configured to emit electromagnetic radiation through an end.

FIG. 27 is a view of a waveguide disposed on an external surface of a tube.

FIG. 28A is a view of a waveguide disposed on an external surface of a tube, at least a portion of the waveguide is in an indentation in the tube.

FIG. 28B is a cross section of FIG. 28A.

FIG. 29A is a view of a longitudinal cross section of a tube having a shaped end with a waveguide disposed along at least a portion of the tube.

FIG. 29B is a view of a longitudinal cross section of a waveguide that is a tube.

FIG. 29C is a view of a longitudinal cross section of a waveguide that is a tube having a shaped end.

FIG. 29D is a view of a longitudinal cross section of a waveguide that is a tube having a shaped end for side firing.

FIG. 29E is a view of the side of a tube having a shaped end having one or more apertures a waveguide is disposed inside the tube along its longitudinal axis.

FIG. 29F is a view of the end of the tube having one or more apertures with the waveguide substantially centered in the tube.

FIG. 29G is a view of a cross section of the tube with the waveguide substantially centered in the tube.

FIG. 29H is a view of the end of the tube having one or more apertures with the waveguide located close to the inside surface of the tube.

FIG. 29I is a view of the end of the tube having one aperture with the waveguide substantially centered in the tube.

FIG. 29J is a view of the end of the tube having with the waveguide disposed on the external surface of the tube.

FIG. 29K is a view of the side of a tube with a side firing waveguide adjacent an external surface of the tube.

FIG. 29L view of the side of a tube with a side firing waveguide adjacent an external surface of the tube.

FIG. 30 is a view of a tube with a closed end, a reflective surface is disposed on an inside surface of the tube substantially opposite an aperture, at least a portion of a waveguide is disposed in the tube.

FIG. 31 is a view of a shaped waveguide enclosed by a tube and surrounded by a fluid inside the tube.

FIG. 32 is another view of a shaped waveguide enclosed by a tube and surrounded by a fluid inside the tube.

FIG. 33a is a view of a waveguide having a distal end adjacent an optical sphere, at least a portion of the waveguide and at least a portion of the optical sphere are surrounded by a buffer.

FIG. 33b is a cross sectional view of a waveguide having a distal end adjacent an optical sphere and surrounded at least in part by a buffer and shown in FIG. 33a.

FIG. 34a is perspective view of a hand piece with a waveguide and a spacer.

FIG. 34b is a view of the junction, fork and the roller of the spacer and the waveguide shown in FIG. 34a.

FIG. 34c is a side view of the junction, fork and the roller of the spacer and the waveguide shown in FIG. 34a.

### DETAILED DESCRIPTION

The system includes a light and laser based device for use in general surgery, plastic surgery and in dermatology. The system can include a medical laser device, more specifically, a laser surgical instrument for use in, for example, general surgery, plastic surgery, and in dermatology. The system and/or the device may be employed to treat soft tissue adjacent the dermal-hypodermal junction with precision and with minimal trauma to the patient's body. Optionally, the system and/or the device may be employed to treat the patient's skin, for example, from within the dermal-hypodermal junction (e.g., beneath the dermis). In other embodiments, the system, the device, and/or additional devices may be employed to treat the patient's skin from the inside (e.g., from within the dermal-hypodermal junction) and from the outside (e.g., employing complimentary non-invasive surface treatments) simultaneously or subsequently. The system, the device, methods employing the system or device may be employed, for example, to melt fat tissue, to remove the melted fat tissue from the patient's body, and to treat skin in the dermal-hypodermal region. Additionally, the system, the device, and methods employing the system or device may be employed, for example, to treat veins including leg veins by, for example, coagulation at suitable wavelengths such as 980 nm and 1440 nm, for example.

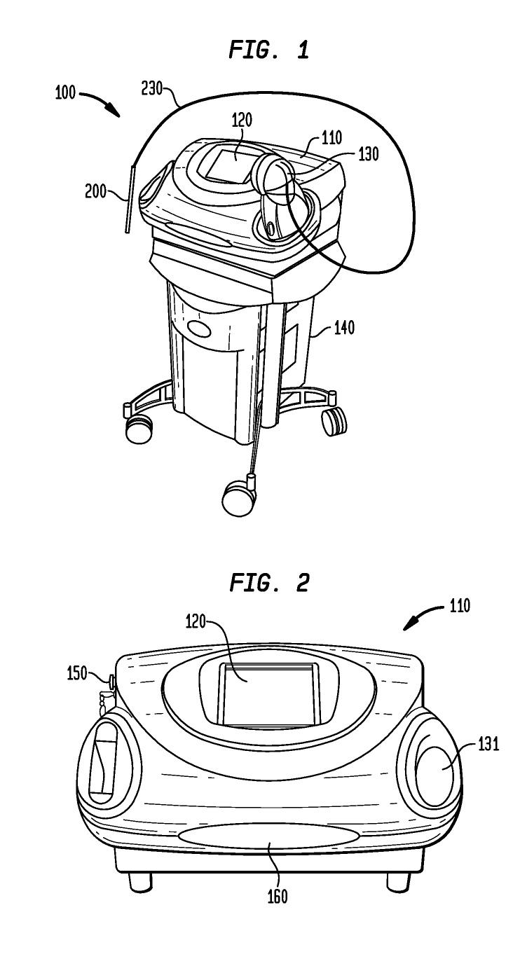

Referring now to FIG. 1, a system 100 is configured to deliver electromagnetic radiation (EMR). The system 100 may be a transportable system that includes one or more of a base unit 110 and a cart 140. The system 100 can include a footswitch (not shown) that actuates EMR delivery. A connection to the actuating footswitch may be located, for example, in the rear portion of the base unit 110. In addition to transporting the system, the cart 140 can optionally provide cooling to the system 100 and/or the base unit 110.

Referring now to FIG. 2, the system's base unit 110 can house a power supply, software, and a user interface panel 120. The base unit 110 includes an emergency stop button 150, a user interface panel 120 (such as, for example, a computer touch screen), a cluster connection site 131, and a software access port 160. A user or practitioner can input the parameters or select from a menu of available parameters (e.g., treatment parameters) on the user interface panel 120.

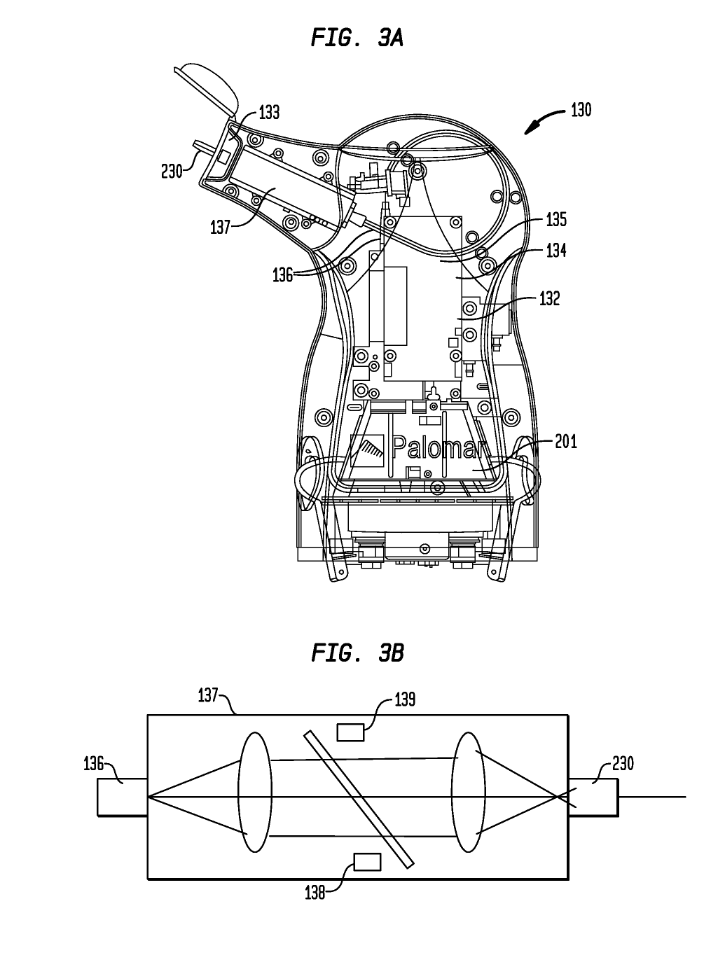

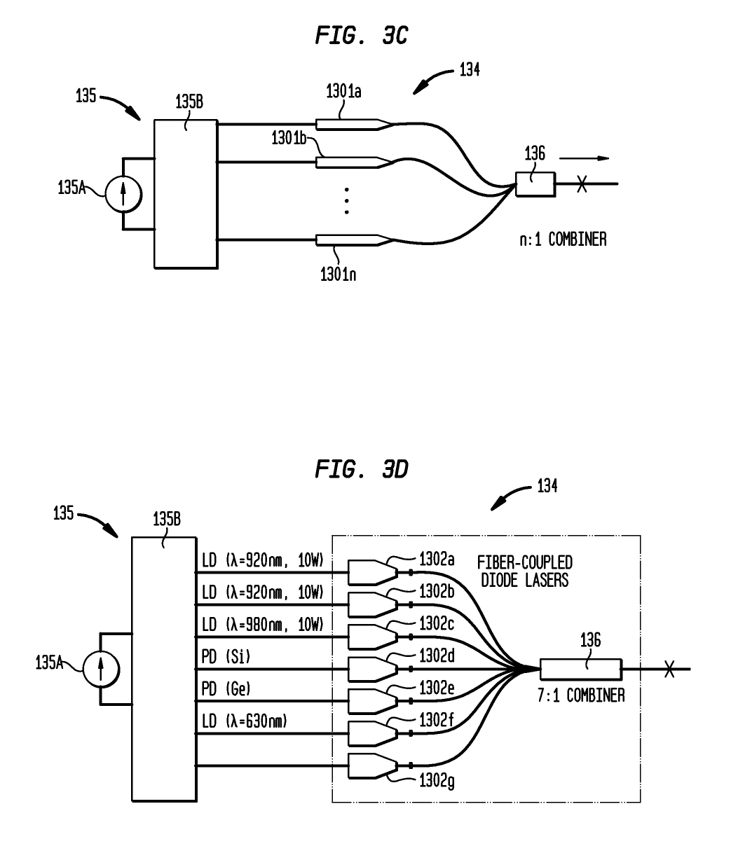

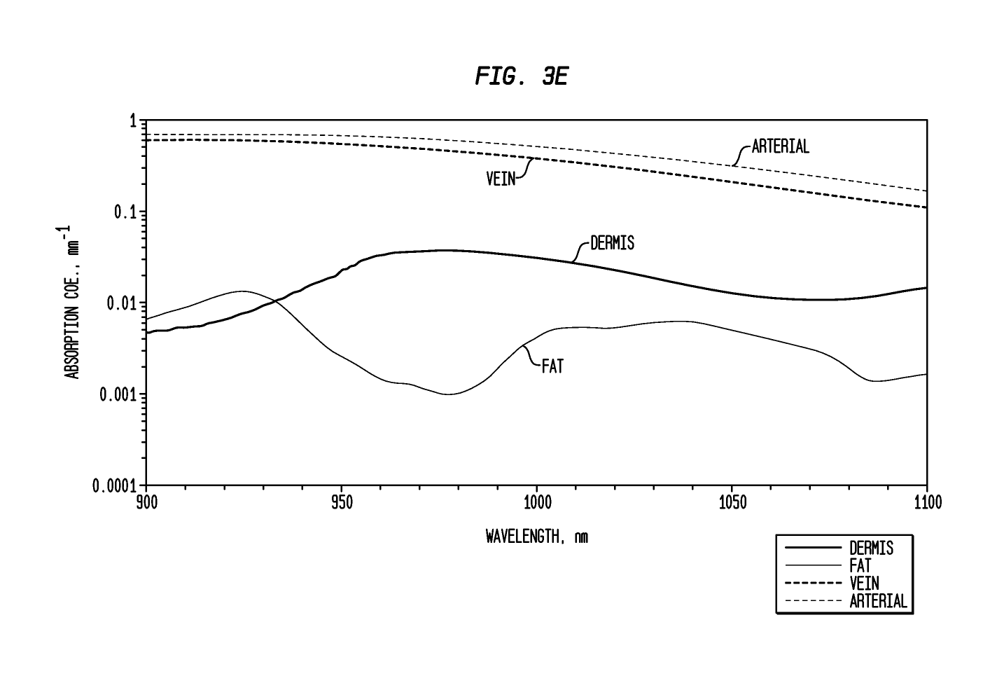

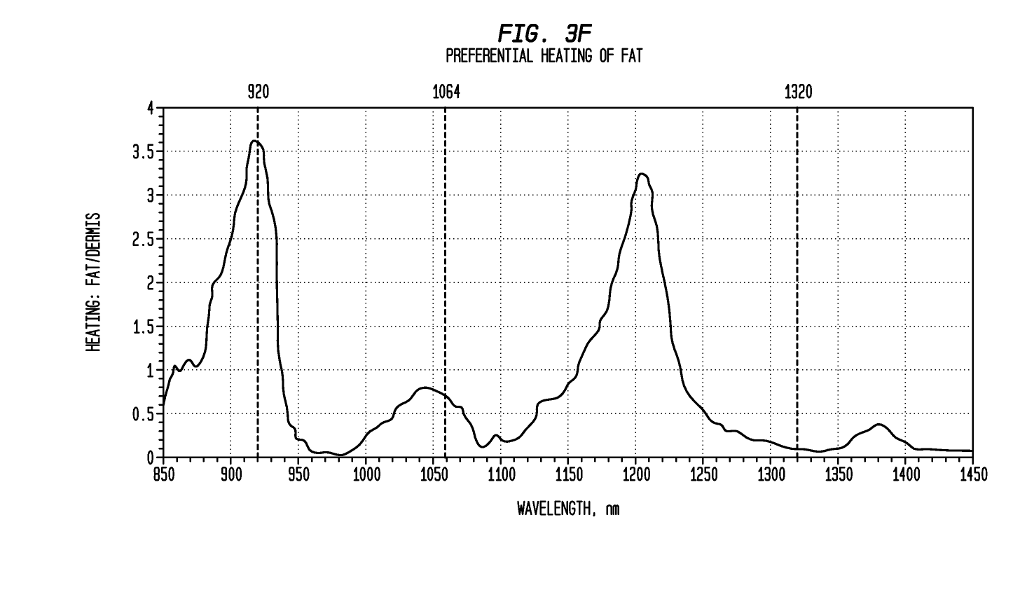

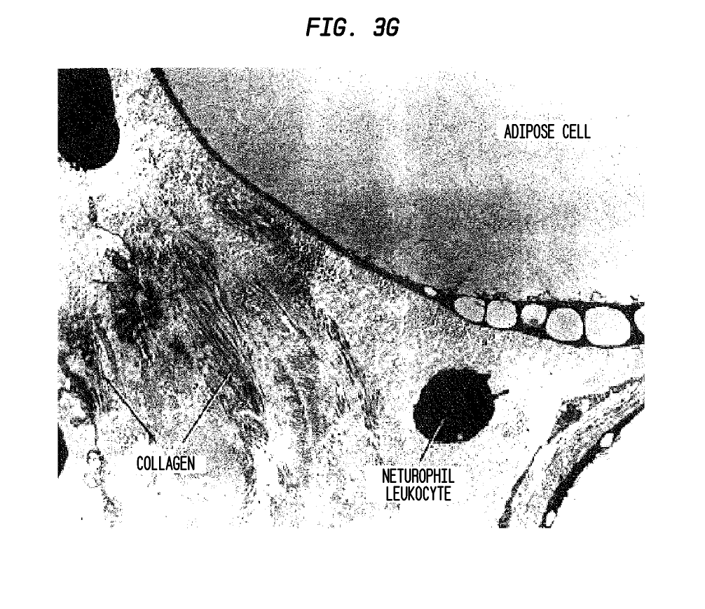

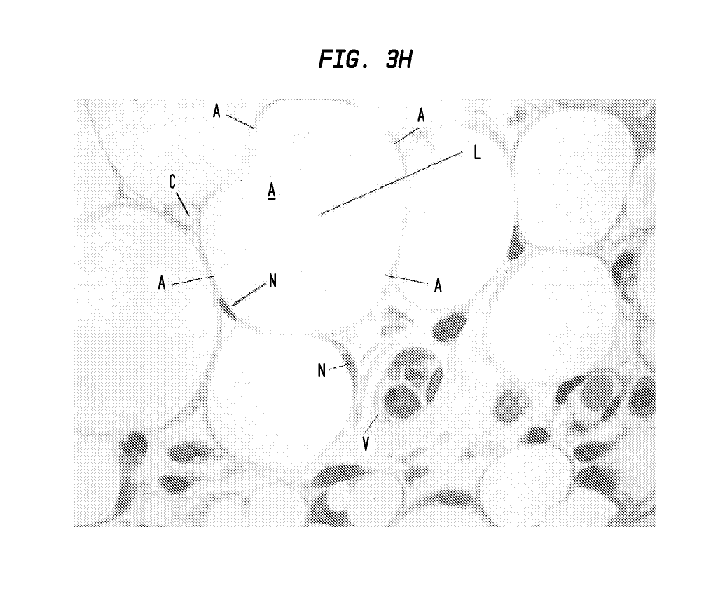

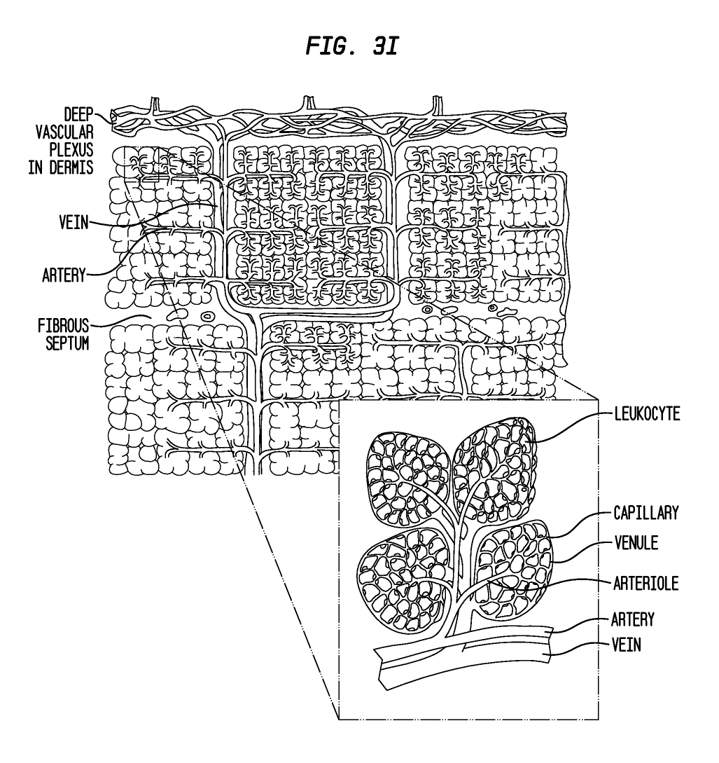

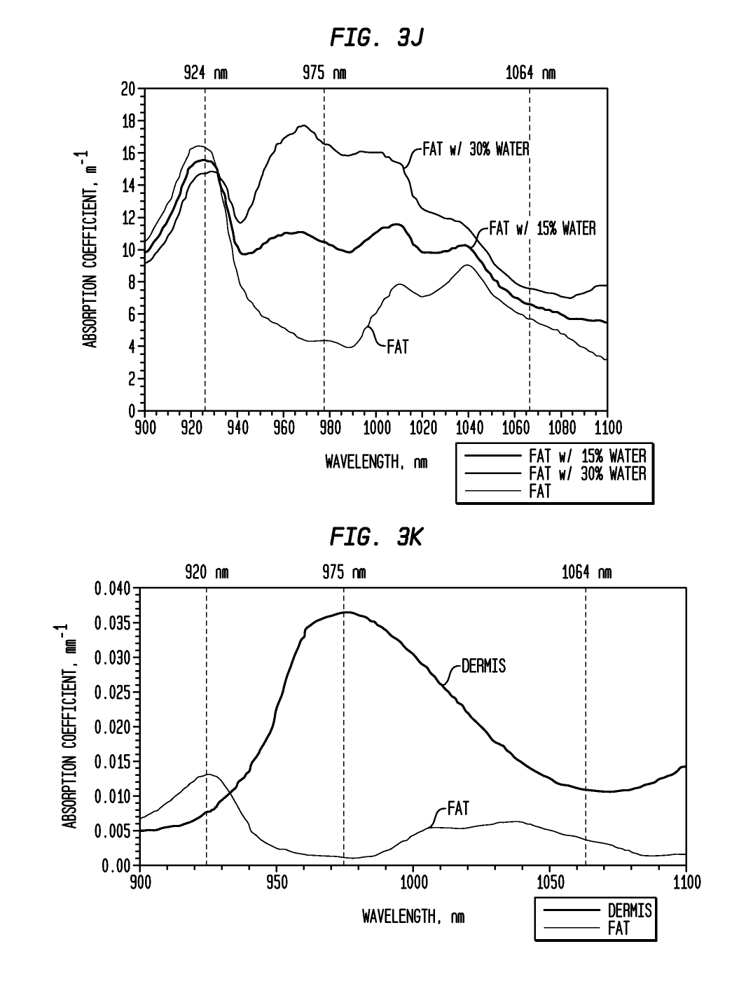

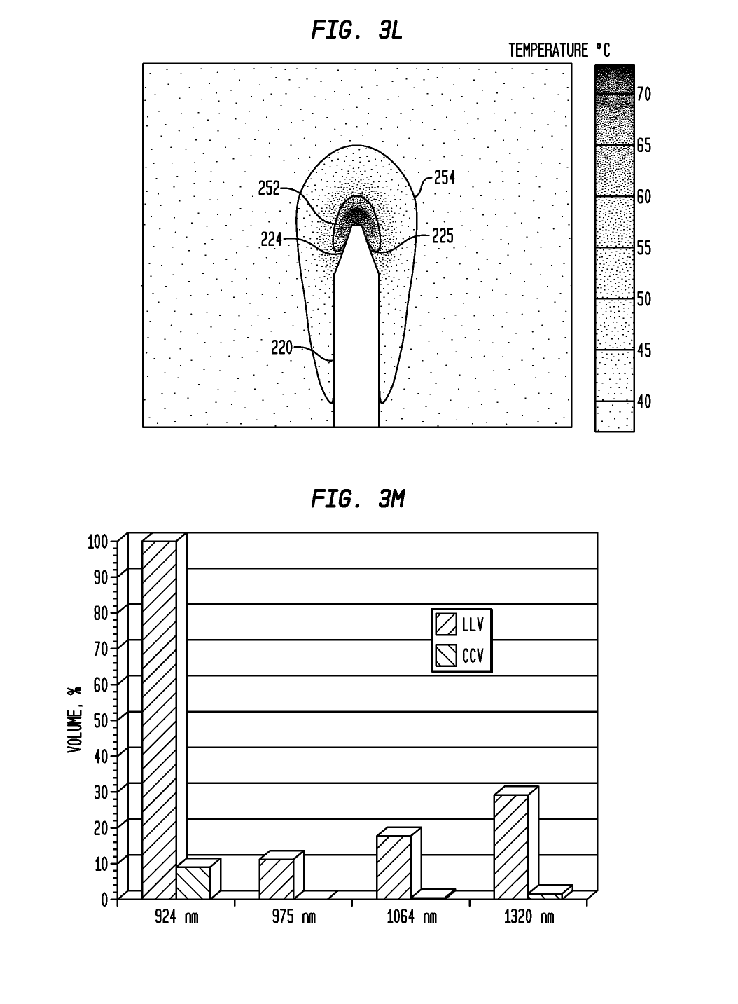

Referring now to FIGS. 1-2, a cluster connector 130 connects to the base unit 110 at the cluster connection site 131. The cluster connector 130 can be removable from the base unit 110. The cluster connector 130 may be made from materials that are commonly found in light and laser systems. Referring now to FIG. 3A, the cluster connector 130 includes an output connector 133 (e.g., an SMA connector) that enables connection of the cluster connector to the hand piece 200 via a fiber 230 (see, e.g., FIG. 1). The output connector 133 can function as an optical fiber connector.

Inside the cluster connector 130 is a source of electromagnetic radiation (EMR), in this case a source of optical radiation such as visible and infrared light (referred to herein as a “light source”). Depending on the treatment to be performed, the light source may be configured to emit a single wavelength, multiple wavelengths, or in one or more wavelength bands. The light source may be a coherent light source, for example, a ruby, alexandrite or other solid state laser, gas laser, diode laser bar or other suitable laser light source. Alternatively, the source may be an incoherent light source for example, an LED, arc lamp, flash lamp, fluorescent lamp, halogen lamp, halide lamp or other suitable lamp. Any of a number of suitable light sources may be employed to provide optical energy to the hand piece 200. Energy from the hand piece 200 exits via the waveguide.

Various light-based devices can be used to deliver doses of EMR to the dermal-hypodermal region. The optical radiation source(s) utilized may provide a power density to the soft tissue of the dermal-hypodermal region of from approximately 25 W/cm² to approximately 1 MW/cm². The power density employed will be such that the targeted therapeutic effect (e.g., adipose tissue liquification, heating of the dermis) can be achieved. The power density will also vary as a function of a number of factors including, but not limited to, the condition being treated, the wavelength or wavelengths employed and the body location where treatment is desired, i.e., the thickness of the skin in the treatment region, the quantity of adipose tissue in the treatment region, the user's skin type, etc. A suitable source may, for example, provide a power of from about 1 W to about 100 W, from about 5 W to about 30 W, or from about 2 W to about 10 W.

Suitable sources include solid state light sources such as:

1. Light Emitting Diodes (LEDs)—these include edge emitting LED (EELED), surface emitting LED (SELED) or high brightness LED (HBLED). The LED can be based on different materials, such as, without limitation, GaN, AlGaN, InGaN, AlInGaN, AlInGaN/AlN, AlInGaN (emitting from 285 nm to 550 nm), GaP, GaP:N, GaAsP, GaAsP:N, AlGaInP (emitting from 550 nm to 660 nm) SiC, GaAs, AlGaAs, BaN, InBaN, (emitting in near infrared and infrared). Another suitable type of LED is an organic LED using polymer as the active material and having a broad spectrum of emission with very low cost.
2. Superluminescent diodes (SLDs)—An SLD can be used as a broad emission spectrum source.
3. Laser diodes (LD)—A laser diode may be the most effective light source (LS). A wave-guide laser diode (WGLD) is very effective but is not optimal due to the difficulty of coupling light into a fiber. A vertical cavity surface emitting laser (VCSEL) may be most effective for fiber coupling for a large area matrix of emitters built on a wafer or other substrate. This can be both energy and cost effective. The same materials used for LED's can be used for diode lasers.

Other laser and non-laser sources may be employed. For example, an Nd:Yag laser may be employed.

In one embodiment, referring to FIG. 3A, a diode laser assembly 134 includes one or more diode laser bar housed within the cluster connector 130. A cooling plate 132 of a heat exchanger is housed in the cluster connector 130 and is made of materials including, for example, copper. The cooling plate 132 maintains a temperature suited to the cluster connector 130 and/or the diode laser assembly 134. A power distribution board 135 can multiplex to select one or more diode laser bar(s) in the diode laser assembly 134 that is powered at a given time. Within the diode laser assembly 134 the diode laser bars convert electrical energy (i.e., in the form of current) into optical energy. Referring now to FIGS. 3A and 3B, the optical energy 136 exits the diode laser assembly 134 and enters a fiber coupler assembly 137. The optical energy 136 is transmitted through the fiber coupler assembly 137. The fiber coupler assembly 137 can include a beam splitter. The optical energy is transmitted through the fiber coupler assembly 137 and the optical energy exits the fiber coupler assembly 137 via the fiber 230, see also FIG. 1.

In one embodiment, the optical energy 136 enters the fiber coupler assembly 137, is reflected off of the beam splitter, and a photodetector 138 receives some of optical energy, converts it to electrical signal thereby enabling communication with the base unit 110 such that the energy generated from the laser source (e.g., the diode laser assembly 134) can be determined. In this way, the energy provided to the hand piece via the cluster connector 130 can be monitored. The fiber coupler assembly 137 can also include a photodetector 139 that monitors one or more temperature at the treatment site thereby enabling feedback control between the hand piece and the cluster connector 130, and/or the base unit to enable energy regulation (e.g., to avoid too little or too much energy) at the treatment site.

Optionally, a controller is disposed in the hand piece 200, the cluster connector, the base unit 110, and/or the system. The controller can be employed independently or in addition to the photodetectors 138 and 139. The controller can be capable of measuring a property, estimating a property, calibrating the device, controlling electromagnetic radiation, or any combination thereof. More specifically, the controller can be capable of: measuring infrared radiation at or near an end of the waveguide, measuring infrared radiation at or near the region of soft tissue, estimating the temperature at or near an end of the waveguide, estimating the temperature in the region of soft tissue, controlling optical radiation generation as a function of the measured infrared radiation, controlling optical radiation generation as a function of the estimated temperature, controlling optical radiation generation as a function of an emissive temperature at an end of the waveguide, calibrating the waveguide, controlling optical radiation generation as a function of thermal activity at the interface of an end of the waveguide and the region of soft tissue, or any combination thereof. The controller may include Ge, InGaAs, two or more diodes or any combination thereof.

Referring now to FIGS. 3A and 3C, the diode laser assembly 134 can include one or more diode laser bar 1301a, 1301b, and 1301n. In one embodiment, the diode laser bars are optically coupled to provide a single fiber of optical energy 136 exiting the diode laser assembly 134. Each diode laser bar, e.g., 1301a, produces a coherent radiation in which all the waves in the individual diode laser bar are at the same frequency (e.g., all the waves are within the same frequency range) when current passes there through. Diode laser bars can have varying wattage limits. For example, the wattage limits can range from about 1 W to about 100 W, or from about 5 W to about 30 W, for example. For example, diode laser bar 1301a can have a frequency of 1060 and a wattage limit of 5 W whereas the diode laser bar 1301b can also having a frequency of 1060 and a wattage limit of 30 W. The number of diode laser bars and the variety of frequencies and wattage levels of the diode laser bars within the diode laser assembly 134 can be selected to enable a variety of treatments using the hand piece.

In one embodiment, referring now to FIG. 1, a user employs the user interface panel 120 to input commands to conduct laser assisted lipolysis. FIG. 3E shows an absorption spectra of fat, dermis, vein, and arterial tissue measured in mm⁻¹ versus wavelength measured in nm. The spectra shows that absorption (e.g., liquefaction or melting) of fat occurs in the range of from about 910 nm to about 940 nm, from about 920 nm to about 930 nm, or about 925 nm. The spectra also show that the absorption (e.g., heating and/or coagulation) of dermis tissue occurs in the range of from about 950 nm to about 1000 nm, from about 960 nm to about 990 nm, or from about 970 nm to about 980 nm. Additionally, vein treatment may be conducted (e.g., to treat leg veins) by coagulation of veins at suitable wavelengths such as 980 nm and 1440 nm, for example. Suitable wavelength for vein treatment may range from about 600 nm to about 1500 nm, for example.

FIG. 3F provides the thermo-optical selectivity of subcutaneous fat compared to dermis. More specifically, the graph compares a ratio of temperature rise of subcutaneous fat to the temperature rise of dermis as a function of a fixed amount of energy at an applied wavelength. In FIG. 3F, the x-axis is the applied wavelength measured in nm and the y-axis is the ratio of the heating of fat (e.g., the temperature rise of subcutaneous fat) over the heating of dermis (e.g., the temperature rise of dermis) when exposed to the applied wavelength of the x-axis. The ratio of the temperature rise of fat to the temperature rise of the dermis has no units. The peak that is shown at from about 860 nm to about 950 nm, or from about 890 to about 940, or from about 910 to about 930 shows that the amount of temperature rise of subcutaneous fat is nearly four times the temperature rise of dermis when exposed to this wavelength range (e.g., from about 910 to about 930). As a result, this wavelength range (e.g., from about 910 to about 930) targets melting or liquefying subcutaneous fat while providing a much less drastic (nearly four times less) temperature change in the dermis. Accordingly, when this wavelength range (e.g., from about 910 to about 930) is applied to subcutaneous fat in the dermal-transdermal region the temperature rise impact on fat is nearly four times the temperature rise impact on the nearby dermis. Thus, preferably, lipid-rich tissues such as subcutaneous fat are treated more efficiently at wavelengths where the ratio is relatively higher, although other wavelengths and combinations of wavelengths could be used.

Referring to FIGS. 3A and 3D, the power distribution board 135 provides a current source 135A that powers a diode laser bar labeled 1302a to produce radiation in the wavelength range including 920 nm at 10 W. Alternatively, the power distribution board 135 provides a current source 135A to power the diode laser bar labeled 1302a to produce radiation in the wavelength range including 920 nm at 5 W and to power the diode laser bar labeled 1302b to produce radiation in the wavelength range including 920 nm at 5 W. The optical energy from the diode laser bar(s) is combined at 136, is transmitted through the fiber coupler assembly, is transmitted through the fiber 230 into the hand piece 200 for treatment of the patient's dermal-hypodermal junction (see, FIG. 1). The user's selected wavelength range, 920 nm, is employed to liquefy adipose tissue.

Optionally, the user also employs the user interface panel 120 to input commands that produce an aiming beam at the tip of the hand piece. The power distribution board 135 provides a current source 135A that powers a diode laser bar labeled 1302f to produce radiation in the visual spectrum wavelength range including 630 nm. The optical energy from the diode laser bar(s) 1302a, 1302b, and 1302f is combined at 136, is such that optical energy is transmitted through the fiber coupler assembly, is transmitted through the fiber 230 into the hand piece 200 for treatment of the patient's dermal-hypodermal junction with the visual aid of an aiming beam (see, FIG. 1).

In another embodiment, in addition to laser assisted lipolysis with the aid of an aiming beam, the user also inputs commands into the interface panel 120 that enable treatment of the dermis tissue at 10 W in the region of the dermal-hypodermal junction. Referring to FIG. 3D, the power distribution board 135 provides a current source 135A that powers a diode laser bar labeled 1302c to produce radiation in the wavelength range including 980 nm at 10 W. Optionally, the two or more wavelength ranges (e.g., 920 nm and 980 nm) blended such that they transmitted sequentially or, optionally, simultaneously.

In another embodiment, the wavelength range and the intensity are blended, for example, where two wavelengths are employed there can be a two diode laser bars producing radiation in the first wavelength range including 920 nm at 10 W for a combined 20 W at 920 nm and the second wavelength range includes 980 nm at 10 W. In this way the wavelength and the intensity is blended.

The optical energy from the diode laser bar(s) 1302a, 1302b, 1302c, and 1302f is combined at 136, such that optical energy is transmitted through the fiber coupler assembly, is transmitted through the fiber 230 into the hand piece 200 to enable treatment soft tissue that liquefies adipose tissue and coagulates dermis in the patient's dermal-hypodermal junction with the visual aid of an aiming beam (see, FIG. 1).

FIGS. 3C and 3D show that the current source 135A is multiplexed via a multiplexer 135B that enables the diode laser bars to be run individually and/or allows multiple diode laser bars to be run simultaneously. Other suitable current sources can be run in series in parallel. Optionally, each laser bar can have a dedicated power supply and/or current source.

Referring again to FIG. 3D, the diode laser assembly 134 can have one or more detectors such as, for example, a temperature feedback detector or a power feedback detectors. For example, the power feedback detector 1302d can be employed to inform the user and/or the system of the amount of power being delivered to the diode laser assembly 134. Power feedback detectors may be made from materials including Silicon, for example. In another embodiment, the temperature feedback detector 1302e can be employed to inform the user and/or the system of the temperature of at least a portion of the hand piece (e.g., the tip) and/or the temperature of a region of soft tissue in contact with a portion of the hand piece. Suitable temperature feedback detectors may be made from materials including Germanium, for example. In another embodiment, the infrared emission feedback detector 1302e can be employed to inform the user and/or the system of the temperature of at least a portion of the hand piece (e.g., the tip) and/or the temperature of a region of soft tissue in contact with a portion of the hand piece. Suitable infrared emission feedback detectors may be made from materials including Germanium, InGaAs, InAs for example. Optionally, a controller disposed in the hand piece 200, the cluster connector, the base unit 110, and/or the system can be employed independently or in addition to feedback detectors (e.g., the power feedback detector 1302d and the temperature feedback detector 1302e and/or the infrared emission feedback detector 1302e). The controller can be capable of measuring a property of the treated tissue, estimating a property (e.g., of the treated tissue), calibrating the device, or controlling electromagnetic radiation, for example.

In accordance with one laser assisted lipolysis treatment one or more treatment regions are pre-treated with Tumescent solution. Tumescent solution contains, among other agents, anesthesia and water. Accordingly, just prior to treatment, the area to be treated has a quantity of water provided via the tumescent solution and a quantity of fat. The 920 nm wavelength primarily heats lipids (e.g., absorption resulting in melting or liquefying all or a portion of a quantity of fat) and the use of 980 nm wavelength primarily heats water (e.g., absorption resulting in heating of a quantity of water from the treatment area). While not being bound to any single theory, the applicant believes that by blending treatments at 920 nm and 980 nm during laser assisted lypolysis interference caused by the presence of water in the treatment area is addressed and/or obviated thereby improving targeted melting and/or removal of fat.

In one embodiment, a controller is configured to receive a temperature signal indicative of the temperature of an external surface of the dermal tissue. The controller emits EMR as a function of the measured temperature of the external surface of the dermal tissue. In this way, when the waveguide treats the dermis tissue overheating can be avoided.

Referring now to FIG. 1, one or more hand piece 200 may be connected to the base unit 110 at the cluster connection site 131 through the cluster connector 130. Suitable hand pieces 200 may be connected to and disconnected from the base unit 110 via a cluster connector 130 located at the cluster connection site 131. Alternatively, the hand piece 200 may be connected to and disconnected from the base unit 110 via a connection to the cluster connector 130 (e.g., the output connector 133). Each hand piece 200 contains an optical energy source such as, for example, a diode based laser located in the system 100, the base unit 110, and/or the cluster connector 130.

EMR energy (e.g., laser energy) can be delivered through the hand piece 200. Energy (e.g., laser energy) may be delivered to a treatment site in a patient's body through the hand piece 200. The targeted tissue treatment or treatments, e.g., the use, of each hand piece 200 may be determined by the hand piece 200 configuration (for placement about or in a region of a subject's body). The targeted tissue treatment or treatments, e.g., the use, of each hand piece 200 may also be determined by the one or more selected wavelength emitted by the energy source through the hand piece 200. For example, one hand piece 200 may have multiple lasers capable of emitting different wavelength ranges while another hand piece 200 has a single laser capable of emitting a single wavelength.

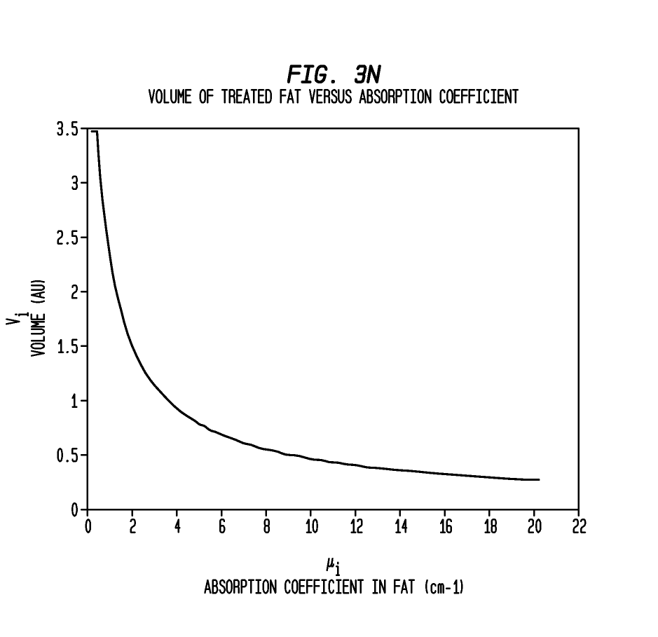

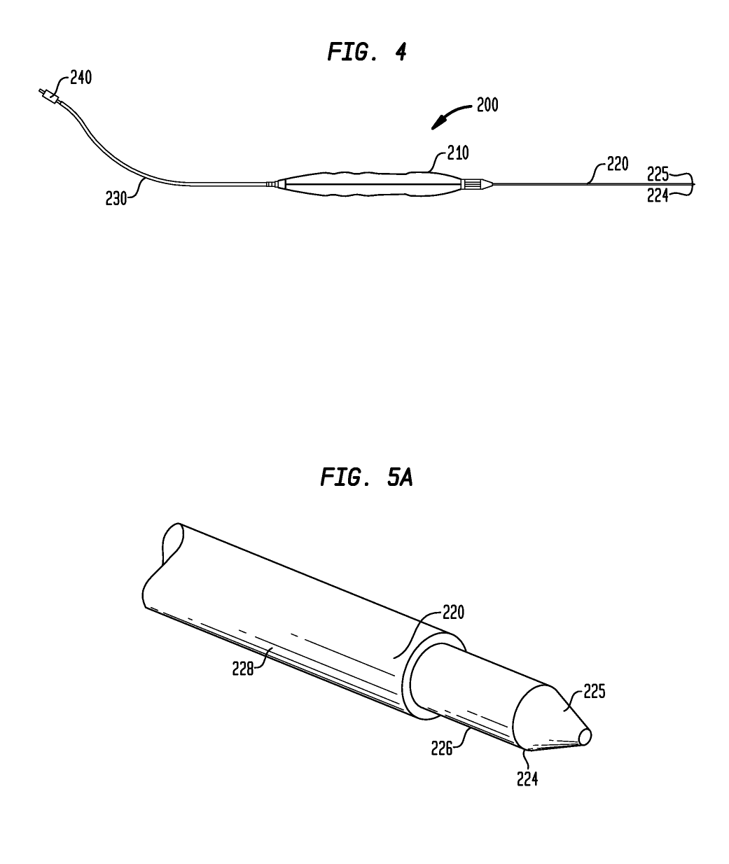

Referring now to FIGS. 1 and 4, in one embodiment, a hand piece 200 is attached to the base unit 110 via a removable cluster connector 130. The hand piece 200 has a waveguide 220 that may be employed to “break-up” or “melt” fat tissue in a subject's body. Methods employing the hand piece 200 to melt fat tissue and/or to remove the melted fat tissue from the patient's body may be referred to as laser assisted lipolysis, laser assisted lipoclasis, laser assisted liposuction, or laser assisted lipectomey, for example. Referring now to FIG. 1, in one embodiment, a hand piece 200 suitable for laser assisted lipolysis is attached to a cluster connector 130 via an optical fiber 230. In other embodiments, the waveguide 220 is employed for hair removal by targeting hair from beneath the dermis at, for example, the dermal-hypodermal junction. In another embodiment, the waveguide 220 is employed beneath the skin to treat nerve and/or muscle tissue to result in decreased nerve and/or muscle activity thereby to reduce or eliminate the appearance of lines and creases on the external surface of the skin. Thus the skin is given a smoother more refreshed appearance.

The disclosed devices and methods of laser assisted liposuction provide laser thermolysis of adipose and/or fat tissue. The device(s) and method(s) disclosed herein are one or more of an easy-to-use device and/or handpiece, a selection of optimized wavelengths and wavelength combinations, efficient and effective power levels, and an efficient and effective tip design. Use of the disclosed devices and methods results in significant hemostasis, tissue shrinkage, easy penetration and heating of adipose tissue. A small cannula can be employed to suction the liquefied tissue. As a result the device provides one or more of easy removal of lipids, a skin shrinkage effect, smoother skin, reduced tissue trauma, reduced patient post-treatment discomfort and bruising, reduced patient downtime, and reduced physician effort and fatigue.

The disclosed devices and methods of laser assisted liposuction disclose selective laser induced melting of tissue, specifically, adipose and/or fat tissue. Adipose tissue is a unique type of connective tissue comprised of adipocytes, blood vessels and fibrous septa and approximately 75-85% lipids and 15-25% water and proteins. The unilocular white or yellow adipose tissue (WAT) is the most common type in adult humans and is composed mostly of mature adipocytes that contain one large droplet of lipids (triglycerides).

The adipocyte's cytoplasm is divided from the surrounding interstitial spaces by the external lamina, a glycoprotein envelope that superficially resembles the basal lamina of epithelia. In addition, the lipid droplet within the cell is not surrounded by a membrane but its interface with the cytoplasm contains a 5-10 nm condensed layer of lipid reinforced by parallel microfilaments 5 nm in diameter. We term this interface Lipid Cytoplasm Interface (LCI, see FIG. 3G).

Adipocytes are surrounded by a loose network of fine reticular fibers containing collagen fibrils, fibroblasts, lymphoid cells, eosinophils and some mast cells (FIGS. 3G and 3H). Adipocytes are well supplied by blood and lymphatic capillaries and appear substantially polyhedral or oval with the nucleus flattened and pushed to the periphery when adiposcytes are grouped together in adipose tissue. Adipocyte's mean diameter depends on the volume of accumulated lipid in the adipocyte cells and ranges from 25 to 125 microns. The volume ratio of lipid to surrounding cytoplasm appears to be high as the cytoplasm is not visible in some areas.

Through laser-assisted lipolysis the desired clinical endpoints of lipid “melting” and removal and in addition the desired tissue retraction and tightening in the treated area are accomplished via lipid removal and controlled tissue coagulation. With these endpoints in mind, the primary goals in designing a system for effective laser thermolysis of fat are choosing the optimal wavelengths, power levels and pulse durations that complement a specific beam and tip geometry.

Referring now to FIG. 4, the waveguide 220 of the handpiece 200 selectively heats adipose tissue to release the intra-cellular lipid from the adipocytes and to coagulate, not tear, the immediate surrounding fibrous support structures. As the lipid temperature rises above a threshold value (e.g., >43° C.), the cells' LCI are irreversibly damaged allowing the lipid to flow into the extracellular compartments, (a process we call lipid liberation or “LL”), for easy suctioning of the fat. Lipid liberation is also referred to herein as “melting” “liquefication” and “break-up” of fat and/or adipose tissue. Continued heating of the adipose tissue to temperatures between 60-70° C. leads to coagulation of local collagenous septa, the network of collagen fibrils surrounding adipocytes, capillaries and blood vessels. This continued heating is primarily via radiation, which is relatively controlled, is secondarily by conduction, and is finally by convection via the melted fat and the heated water in the region of treatment. A clinically beneficial shrinkage effect arises with coagulation of the collagenous tissue. The result is the creation of a channel through the adipose tissue of free-flowing lipid surrounded by a wall of shrunken coagulated fibrous tissue.

The method by which the tissue surrounding the tip is heated is crucial. Power levels sufficient to vaporize lipids or water are significantly greater than the power levels needed for lipid liberation and coagulation of surrounding cells and collagenous tissue. When operating at the high levels required to vaporize lipids and/or water excess energy deposition can occur that not only lowers efficiency but is highly unsafe particularly in regions proximal to the dermis. To most effectively and safely disrupt the adipocytes, free the entrapped lipid and coagulate the surrounding cells and collagenous structures, it is best to perform smooth heating with CW (continuous wattage) or long pulses without overheating to the point of bubble formation or mechanical shock wave generation. In this way, tissue retraction and tightening can most effectively and safely be reached.

In standard liposuction, significant force is needed to cut through the septa and other supporting structures. This force can be transmitted through the septa to distant parts of the adipose tissue and has the potential to cause additional trauma. However, in laser-assisted liposuction with the waveguide 220 of the handpiece 200, the required mechanical force is greatly reduced both by the tip design and by the easy penetration of the coagulated septa and collagen fibrils. The distant infrastructure of the tissue is left substantially intact and substantially undisturbed to support faster healing. An additional benefit of the disclosed device is that smaller cannulae may be used for suctioning as the detached tissue includes freed lipids that have been heated.

FIG. 3J shows several candidate wavelengths at the peaks of the absorption profile for human fat. The figure shows the absorption coefficient in m⁻¹ as a function of a range of wavelengths measured in nm. As shown, a wavelength of from about 910 nm to about 930 nm, or about 924 nm corresponds to an absorption peak of lipid. Lipid is the dominate chromophore in subcutaneous fat, accordingly, a wavelength of from about 910 nm to about 930 nm, or a wavelength of 924 nm was selected to target normal adipose tissue. One or more wavelength in this range provides the maximum selectivity for fat while simultaneously providing sufficient optical penetration into the fat for maximal volume heating of the adipose tissue surrounding the tip. Referring still to FIG. 3J one of the shown peaks corresponds to data related to regular human fat under physiologic conditions, another peak corresponds to regular human fat mixed with 15% water, and the other peak corresponds to data related to regular human fat mixed with 30% water (e.g., the samples mixed with water can act as a surrogate for fat that has been tumesced by a tumescent solution as is typically used in a traditional liposuction and/or laser liposuction treatment).

FIG. 3K shows the Absorption spectrum, measured by the absorption coefficient in m⁻¹, of dermis with 70% water and fat under physiologic conditions versus the applied wavelength measured in nm. The wavelength range of from about 970 m to about 980 nm, or about 975 nm corresponds to a peak in dermal absorption where water is the primary chromophore. Thus, the wavelength range of from about 970 nm to about 980 nm, or about 975 nm targets the dermis for skin tightening by, for example, coagulation of tissue by application of at least one wavelength in the aforementioned wavelength range.

By mixing the wavelength range of from about 970 nm to about 980 nm, or about 975 nm with a wavelength of from about 910 nm to about 930 nm, or a wavelength of 924 nm in a mixed delivery mode, the waveguide tip performance can benefit from use in hydrated adipose tissue following tumescence.

In addition to the applied wavelength(s), another mechanism for heating the surrounding collagen structures in the adipose tissue is through thermal conduction from the neighboring lipid droplets heated by the laser light.

FIG. 3L shows in grayscale a computer model of a typical temperature profile at the distal end 224 of the waveguide 220 including the tip 225, having a substantially bullet or substantially parabolic shape, disposed in adipose tissue. The model depicts the waveguide 220 and its tip 225 at the end of a one second pulse inside the adipose tissue.

Two damage zones are outlined surrounding the tip 225. The larger zone outlines the volume of liberated lipid 254. The smaller zone outlines the volume of protein coagulation 252. The larger lipid liberation volume 254 is characterized by temperatures exceeding about 43° C. and the protein coagulation volume 252 is characterized by temperatures exceeding about 60° C. The coagulated protein in the volume 252 includes, for example, coagulated collagen. The computer model of FIG. 3L assumes that 25 Watts of power were provided in a mixed mode featuring two wavelengths, and at least one within the range of from about 910 nm to about 930 nm, or about 924 nm and the other wavelength within the range of from about 970 nm to about 980 nm, or about 975 nm. If tumescent solution were added to the adipose tissue of the model, the volume of the liberated lipid 254 versus the volume of protein coagulation 252 will be expected to vary depending on, for example, exposure of the tissue to a single wavelength, a mixed wavelength, and/or the relative power for each of the individual wavelengths when treated in a mixed wavelength mode.

FIG. 3M shows the volume percentage of liberated lipid volume (LLV) and the volume percentage of coagulated collagen volume (CCV) in adipose tissue in normal physiologic condition (i.e., adipose tissue absent of hydration due to tumescence) versus the applied wavelength to achieve the volume. For the wavelengths applied the largest relative volume of liberated lipid was realized at a wavelength including 924 nm (i.e., a wavelength range of from about 910 nm to about 935 nm).

When a radiation wavelength range of from about 910 nm to about 935 nm is applied for a period of time and at a power level to liberate lipid from the adipose tissue and at a temperature of the irradiated adipose tissue below the temperature at which all or a portion of the irradiated adipose tissue vaporizes the volume percentage of liberated lipid is at least two times the volume percentage of lipid liberated by an applied wavelength range including a wavelength selective to lipid chromophores not including about 910 nm to about 935 nm applied for the period of time, at the power level and below the temperature. The wavelengths selective to lipid chromophores include from about 910 nm to about 935 nm, from about 1200 nm to about 1230 nm, from about 1700 to about 1740 nm or from about 2300 to about 2320 nm.

The approximate expression for the volume of fat (e.g., adipose tissue) treated (e.g., melted or lipid liberated) with a radiation is given by

$$V = Ax = \frac{ln ( \frac{Δ {T}_{o}}{Δ {T}_{m}} )}{μ} = \frac{ln ( \frac{μ τ {I}_{o}}{ρ {C}_{p} Δ {T}_{m}} )}{μ} = \frac{ln ( c μ )}{μ}$$

### Where:

ΔTₒ is the temperature rise at the tip.
ΔTₘ is the desired melting temperature minus starting temperature (55-37° C.) at a distance x from the tip.
μ is the absorption coefficient of lipids.
ρ is the density of fat (e.g., adipose tissue).
τ is the time the laser is irradiated.
Iₒ is the laser intensity at the tip.
Cₚ is the specific heat of the fat.
Note that absorption of fat at 924 nm is 0.18. We select ΔTₒ to be 100° C.-37° C. (or about 63° C.).

FIG. 3M shows a generalized plot of the volume of fat (e.g., adipose tissue) treated (e.g., melted or lipid liberated) with a radiation is given by the above formula. The FIG. 3M shows generally how the volume of treated fat will change as a function of the absorption coefficient in fat. The absorption coefficient in fat varies with wavelength. In order to maximize the volume of liberated lipid from fat it is desirable to employ the lowest absorption coefficient possible that is selective for lipids.

Referring now to FIG. 4 the hand piece 200 includes a handle 210; at one end of the handle 210 is a waveguide 220 and at the other end of the handle 210 is an optical fiber 230. The hand piece 200 may be disposable after a single use. In one embodiment, the hand piece 200 delivers light to the treatment area through a disposable hand piece. Alternatively, the hand piece 200 can be suitable for a limited number of uses and sterilizations. Optionally, the hand piece 200 can be a durable device suitable for many repeated uses and sterilizations. In one embodiment, the waveguide 220 is disposable after a single use. In another embodiment, the waveguide 220 is suitable for a limited number of uses and subsequent sterilizations. In still another embodiment, the waveguide 220 is a durable device suitable for many repeated uses and sterilizations. In one embodiment, the fiber 230 is disposable after a single use. In another embodiment, the fiber 230 is suitable for a limited number of uses and subsequent sterilizations. In still another embodiment, the fiber 230 is a durable device suitable for many repeated uses and sterilizations. In one embodiment, all or a portion of the hand piece 200 is sterilized by gamma irradiation, ethylene oxide sterilization, or sterilization by other gas or radiation means. Optionally, the hand piece 200 is packaged (e.g., in paper, cardboard, plastic or a combination of these) and ready for sale to the end user prior to sterilization. In another embodiment, the hand piece 200 is sterilized by any suitable means prior to packaging for end user sale. Suitable sterilization methods can include, for example, autoclaving. Autoclaving may be optionally employed between treatments. Ethylene oxide sterilization is a known method of sterilization, which is desirable at least because it does not diminish and/or does not substantially diminish the tensile strength of at least a portion of the hand piece 200 (e.g., the waveguide 220 and/or the optical fiber 230), accordingly, the risk of unwanted or early waveguide 220 and/or optical fiber 230 failure is diminished.

The waveguide 220 can have a diameter that ranges from about 200 μm to about 1500 μm, from about 400 μm to about 800 μm, or from about 800 μm to about 1100 μm. In some embodiments, the waveguide 220 is a fiber that has little stiffness and may have a support and/or bonding with other materials to maintain its rigidity. Referring also to FIG. 5A, the waveguide 220 can be substantially rod shaped. The waveguide 220 can be, for example, a quartz rod. The waveguide 220 can be fabricated from materials including quartz, sapphire, glass, or a combination of quartz, sapphire, or glass, for example. In one embodiment, the waveguide 220 has a core 226 that is a quartz optical fiber and the waveguide 220 has a coating referred to as a buffer 228. The waveguide 220 core 226 can be fabricated from quartz, sapphire, glass, or a combination of quartz, sapphire, and glass for example. The buffer 228 can be made from any of a variety of materials including, for example, a fluoropolymer such as (e.g., Tefzel® or Teflon®), sapphire, quartz, glass or any combination thereof. A sheath or buffer 228 is sized to surround at least a portion of the waveguide 220 and/or at least a portion of the waveguide core 226. In one embodiment, a buffer 228 coating is extruded over the core 226 to conform to and mold onto the core 226. Other suitable core 226 materials and other suitable buffer 228 coating materials are found in other known medical laser products and in procedures that alter or vaporize body tissue. In one embodiment, the buffer 228 provides a safety feature in the event of a waveguide 220 failure. Certain waveguide 220 core 226 materials include quartz, sapphire, glass or combination(s) thereof. In use if the core 226 were to break and/or shatter it could break into many small pieces that would be difficult to locate inside the subject's body. In addition, many core 226 materials such as quartz and glass are translucent and would be particularly difficult to visually locate in a surgical site. Certain buffers 228 that surround the waveguide 220 core 226 maintain their integrity in such a manner that when a core 226 material breaks and/or shatters the buffer 228 material contains the broken pieces thereby avoiding and/or lessening the risk of a foreign body remaining in a subject's body after a waveguide 220 failure. For example, the buffer 228 can be a coating extruded over the core 226 that at least substantially maintains its integrity such that if the core 226 breaks and/or shatters the buffer 228 retains the pieces of the core 226 such that they don't get lost in the subject's body.

A tip 225 can be disposed at the distal end 224 of the waveguide 220, see e.g., FIG. 5A. The distal end 244 and/or the tip 225 can be shaped, tapered, and in one embodiment is bullet shaped. The tip 225 shape can determine where the optical energy focuses. For example, referring now to FIGS. 5B-5E, the shape of the tip can be selected so that the optical energy focuses in a desired location. For example, FIG. 5B shows a tip 225A having a bullet shape that is substantially parabolic. FIG. 5C shows a raytrace of a predicted optical energy pattern emitted from the tip 225A into air. As seen in FIG. 5B, the optical energy of the bullet or parabolic shaped tip 225A focuses at point F, which is inside the tip 225A. It appears that the focusing of light inside the tip is due to the total internal reflectance on the parabolic surface of the bullet shaped tip 225A. FIG. 5D shows a substantially parabolic or bullet shaped tip 225B having a cut end. FIG. 5E shows a raytrace of a predicted optical energy pattern emitted from the cut tip 225B into air. As seen in FIG. 5E, the optical energy of the cut tip 225B focuses at point F, which is outside of the tip 225 and is therefore the optical energy is focused in the air.

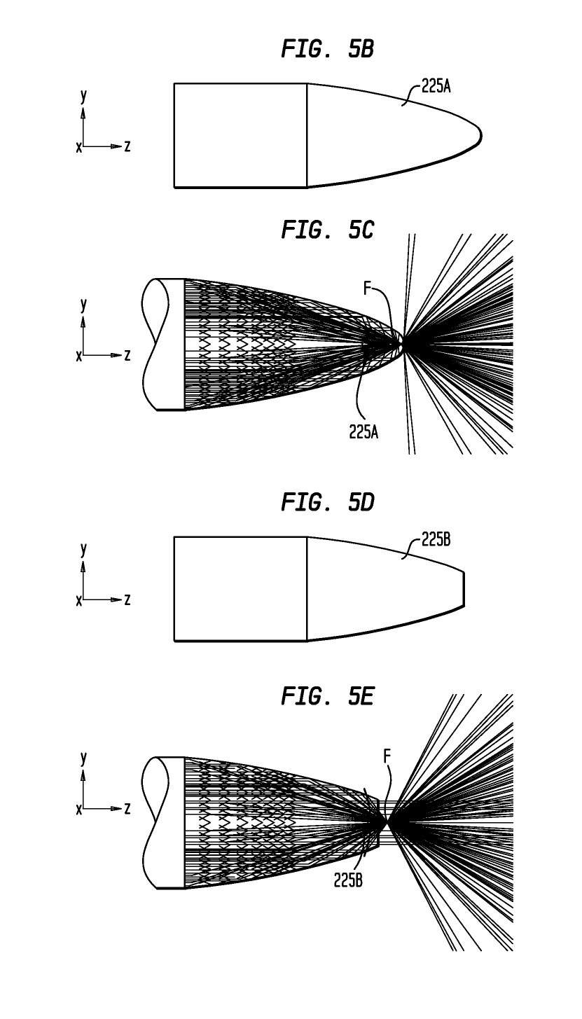

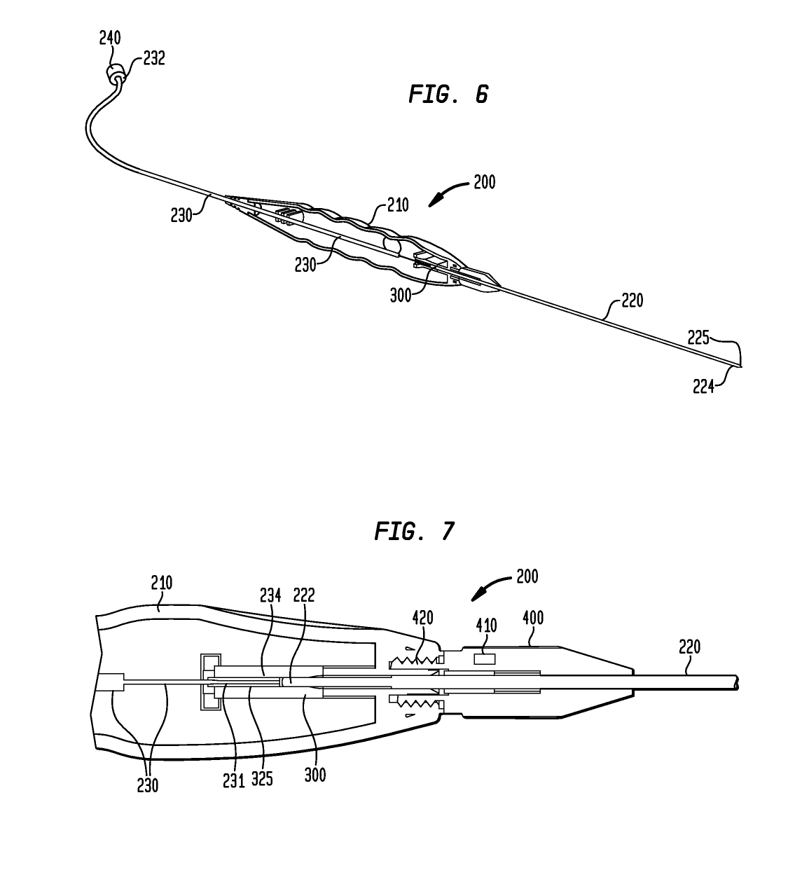

Referring now to FIG. 6, the hand piece 200 has a handle 210 and the handle 210 may be hollow, semi-solid, or solid, for example. The handle 210 may be made using injection molded plastic. The hand piece 200 includes an optical fiber 230. The proximal end 232 of the optical fiber 230 couples to source of EMR (e.g., the base unit 110 or cluster connector 130, see FIG. 1). In one embodiment, a proximal end 232 of the optical fiber 230 has an input connector 240 adapted to connect to a connector disposed on a base unit 110 housing a source of electromagnetic radiation and/or a cluster connector 130.

In one embodiment, an input connector 240 is attached to the proximal end 232 of the optical fiber 230. The input connector 240 is connects to the base unit 110 via the cluster connector 130. The input connector can be, for example, an SMA connector. For example, the input connector 240 connects to the output connector 133 disposed on the cluster connector 130. Alternatively, the input connector 240 connects to base unit 110 at, for example, the cluster connection site 131. In one embodiment, the input connector 240 acts as an interface that connects the hand piece 200 to a source of EMR. The input connector 240 can connect the waveguide 220 to the source of EMR. Optionally, the input connector 240 and/or the output connector 133 include a material that degrades when coupled multiple times. In one embodiment, when the input connector 240 and the output 133 are decoupled the material in at least one of the input connector 240 and the output connector 133 degrades.

Optionally, the cluster connector 130 includes a material that degrades when coupled multiple times. In one embodiment, when the cluster connector 130 is decoupled the material in at least one of the cluster connector 130 and the cluster connection site 131 degrades. In one embodiment, at least one of the cluster connector 130, the input connector 240, or the output connector 133 has a locking mechanism adapted to prevent waveguide 220 replacement or a locking mechanism adapted to prevent coupling input connector 240, the output connector 133, or the cluster connector 130 multiple times.

The optical fiber 230 comes into contact with the waveguide 220 thereby to transport EMR from the output connector 133, through the input connector 240, through the optical fiber 230, and then through the waveguide 220. In some embodiments, the optical fiber 230 and the waveguide 220 are in optical contact, in other embodiments, the optical fiber 230 and the waveguide 220 are in both optical contact and in physical contact. In some embodiments, the optical fiber 230 contacts the waveguide 220 in a location external to the handle 210 (not shown). In other embodiments, the optical fiber 230 connects to the waveguide 220 within the handle 210 of the hand piece 200. The optical fiber 230 can be referred to as an umbilical fiber.

The optical fiber 230 can connect to the waveguide 220 by any suitable connection means. Referring now to FIGS. 7 and 9, suitable means of contacting the optical fiber 230 to the waveguide 220 include, for example, one or more of an adaptor 300 and/or a bushing 325 and/or a ferrule and/or a connector that bring the optical fiber 230 and the waveguide 220 into optical contact and/or alignment. The adaptor 300 and/or a bushing 325 can be sized to cause the optical fiber 230 and the waveguide 220 to be in both physical contact and in optical alignment. One or more of a ferrule, an adaptor 300, and/or a bushing 325 may be housed within the handle 210, or optionally, exterior to the handle 210 (not shown). In one embodiment, a portion of an exterior sheath is removed from the optical fiber 230 creating an exposed portion 231 of quartz. The exterior sheath may be made from Teflon®, nylon, PVC, polyimide, armor jacket material (e.g., metal), for example. At least a portion of the exposed portion 231 of the optical fiber 230 is surrounded by a bushing 325. For example, the optical fiber 230 distal end 234 is surrounded by a bushing 325 or a ferrule. The bushing 325 may be made from any of a number of materials including glass, ceramic, or metal, for example. In one embodiment, the glass bushing 325 is heated to fuse with the exposed portion 231 of the quartz optical fiber. The distal end 234 of the optical fiber 230 together with at least a portion of the bushing 325 is inserted into a first side 301 of an adaptor 300. The adaptor 300 may be made from any of a number of materials including, for example, glass, ceramic, or metal, for example. The proximal end 222 of the waveguide 220 is inserted into the second side 302 of the adaptor 300. In one embodiment, the distal end 234 of the optical fiber 230 and the proximal end 222 of the waveguide 220 are optically aligned within the adaptor 300. The proximal end 222 of the waveguide 220 is disposed in one side of an adaptor 300 and the proximal end 222 is adjacent to a distal end 234 of a fiber 230 disposed in the other side of the adaptor 300.

In another embodiment, the distal end 234 of the optical fiber 230 and the proximal end 222 of the waveguide 220 are in contact, e.g., in physical contact and optically aligned within the adaptor 300. In still another embodiment, the distal end 234 of the optical fiber 230 and the proximal end 222 of the waveguide 220 are optically aligned and are not in physical contact, but are separated by a pre-determined amount of space within the adaptor 300. Optionally, the proximal end 222 of the waveguide and the distal end 234 of the optical fiber 230 are bonded by an adhesive disposed there between. Suitable adhesives include, for example, acrylic adhesive and ceramic adhesive, for example. In another embodiment, the distal end 234 of the optical fiber 230 is fused to the proximal end 222 of the waveguide 220 by, for example, heat. The adaptor 300 can surround at least a portion of one or both of the optical fiber 230 and the waveguide 220 and can be fused in place, for example, to maintain the optical fiber 230 and the waveguide 220 in alignment (e.g., optically and/or mechanically aligned). The adaptor 300 can mechanically align the waveguide 220 and the fiber 230. The adaptor 300 may be disposed within the hand piece 200, e.g., within the handle 210 of the hand piece 200.

Referring now to FIGS. 6, 7 and 8A, a handle cap 400 surrounds at least a portion of the waveguide 200. A portion of the handle cap 400 is a handle connector 420 that connects the handle cap 400 to the handle 210. The handle connector 420 can be connected to the handle 210 by any suitable means. For example, the handle connector 420 can be a screw with threads that mate with threads disposed on or in the handle 210. The handle connector 420 can tension fit, snap fit, or lock fit with the handle 210, for example. The handle connector 420 can be connected to the handle 210 by adhesive, clips, or other joining methods known in the art. Suitable handle connectors 420 can be removed after connection.

The handle cap 400 can be made of a material suited for contact with a subject's soft tissue. All or a portion of the handle cap 400 can be sized or shaped to contact soft tissue in a subject's body. For example, a portion of the handle cap 400 is angled. In another example, a portion of the handle cap 400 has a substantially triangular shape. The handle cap 400 can have a substantially parabolic shape.

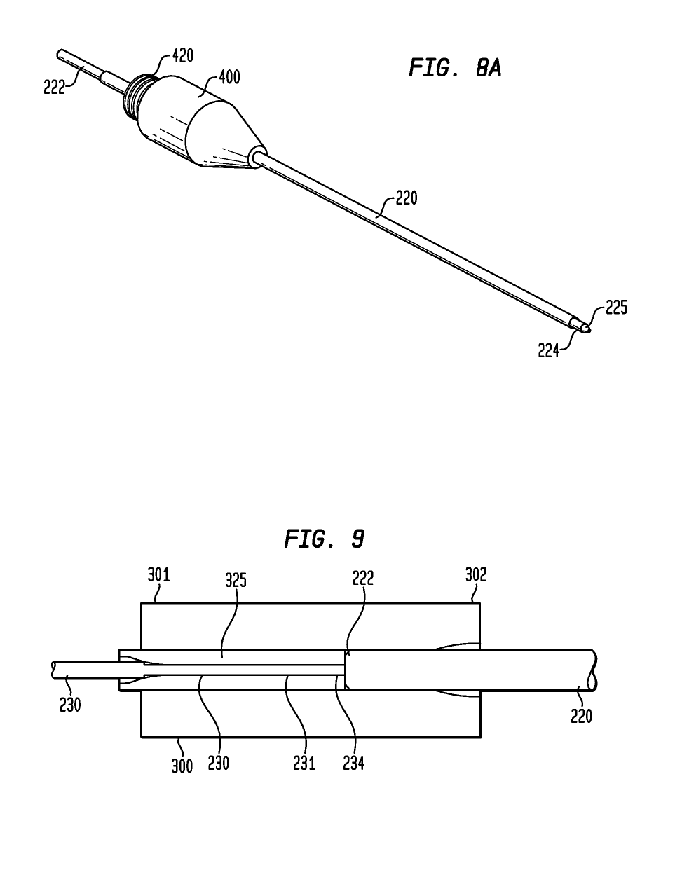

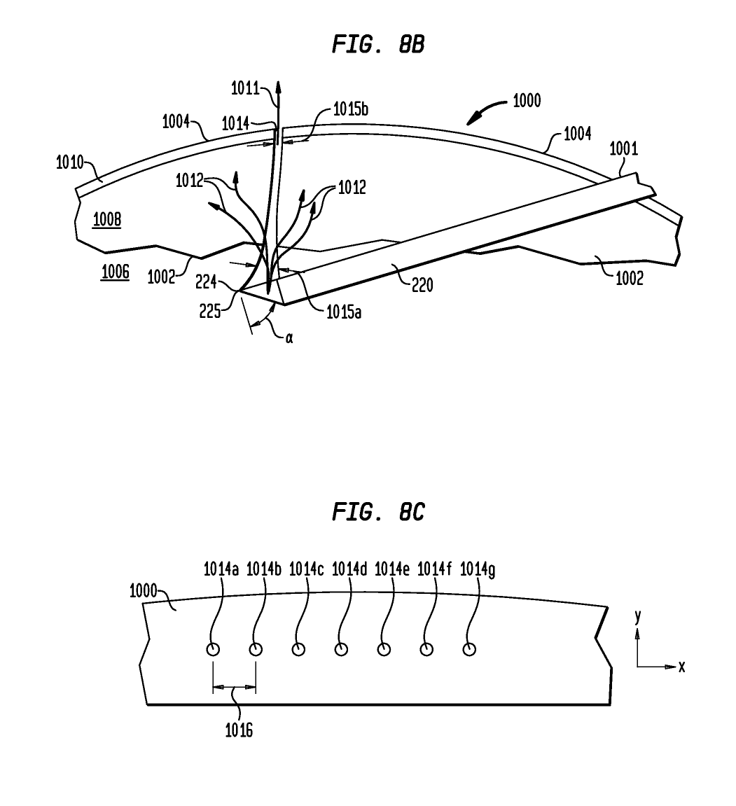

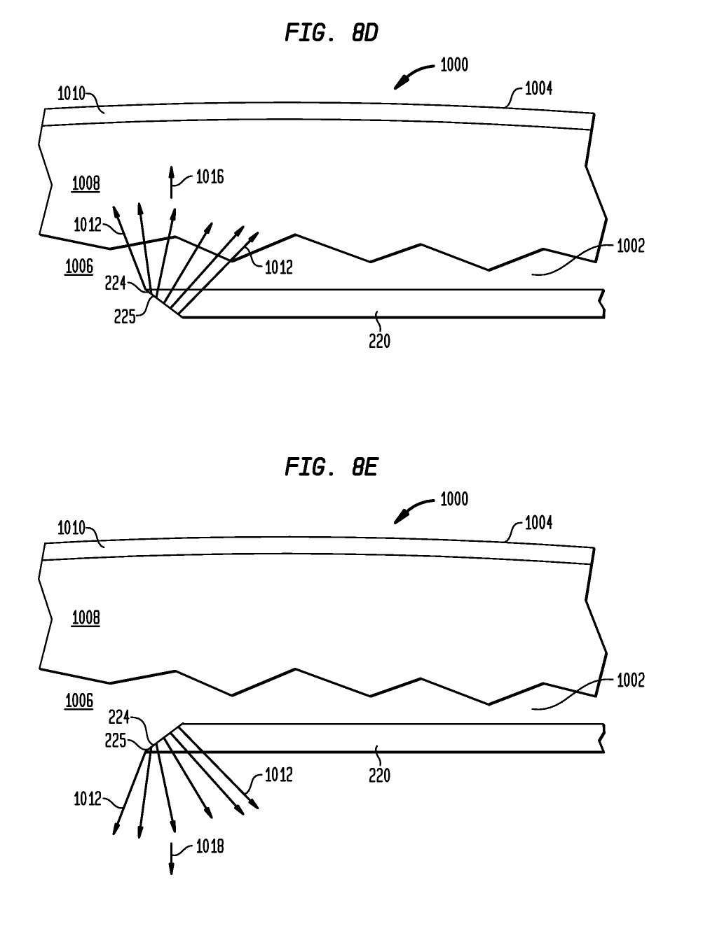

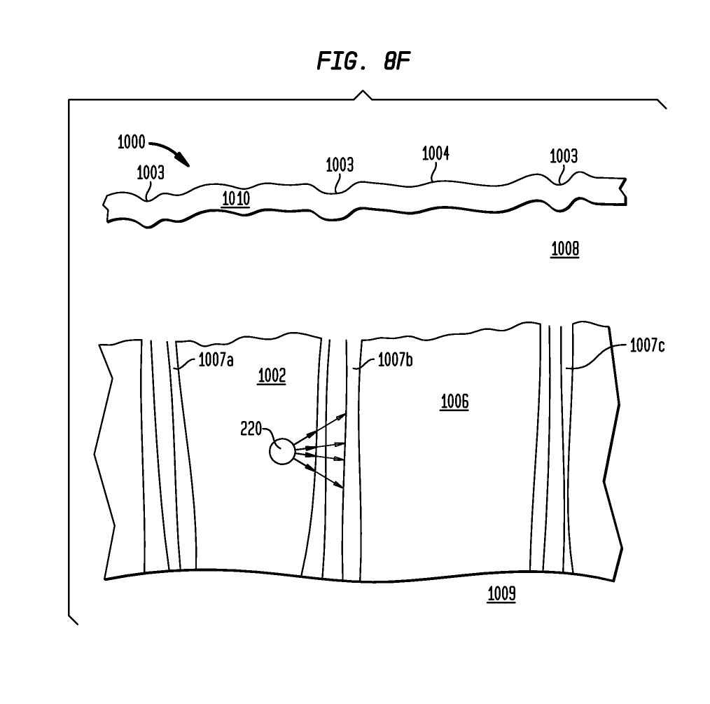

A handle cap 400 surrounding a waveguide 220 can be disposable after a single use. In another embodiment, a handle cap 400 can be reused a limited number of times enabling a number of sterilization cycles between uses by, for example, autoclave, gamma irradiation and/or ethylene oxide sterilization. Referring again to FIG. 8A, a handle cap 400 can be coated with a material that provides insulation to prevent excess heat from contacting the tissue of the subject. Alternatively, cover can be employed that complements all or a portion of the handle cap 400 shape. Suitable covers can be made from any of a number of materials including, for example, polymer, silicon, or any other materials that can at least partially or possibly totally block heat from the handle cap 400 from contacting the subject's tissue. In this way, the coating and/or the cover over the handle cap 400 can prevent all or a portion of the heat from the handle cap 400 from contacting the patient. The cover and/or coating is maintained over the handle cap 400 during a treatment procedure. In this way, the patient's tissue can be protected from unwanted and/or unintended heat contact.

In addition, a removable cover can be employed to protect the waveguide proximal end 222 from, for example, damage. Suitable removable covers that can be employed to protect the waveguide proximal end 222 may be made from materials including, for example, metal, plastic, rubber, paper, or any combination thereof. Such removable covers may be placed on the waveguide proximal end 222 prior to packaging and remain in place until the end user removes the removable cover when connecting the waveguide 200 to the hand piece 220 via the waveguide proximal end 222.

A control tag 410 can be disposed on a handle cap 400 or on a waveguide 220 (see, e.g., FIG. 7) or an a package that contains the hand piece 200. A control tag 410 can be employed to enable and/or to disable energy flow to the waveguide 220. In one embodiment, a control tag 410 is programmed to limit the number of times a waveguide 220 can be used and therefore to limit the number of treatments conducted on various subjects or various body regions using a single waveguide 220. In some embodiments, a control tag 410 is programmed to set a quantity of time that a single waveguide 220 can be used; in this way the control tag 410 can be employed to limit the waveguide 220 such that it is only used on a single patient. Suitable time limits that may be programmed into a control tag 410 range from about 30 minutes to about 20 hours, from about 1 hour to about 8 hours, or from about 4 hours to about 8 hours. Suitable control tags 410 include recognition system, for example, RFID tags, RFID transponders, bar codes, or holograms that identify the waveguide 220 together with the handle cap 400. In still another embodiment, a control tag 410 is disposed on a package containing all or a portion of the handpiece 200. In this way, the control tag 410 can be employed to activate the handpiece 200 either inside or outside a sterile field of use. For example, in one embodiment, a package bearing a control tag 410 and holding a sterile handpiece 200 is brought into proximity with a receiver disposed on the base unit 110. A control tag receiver or a control tag transponder communicates with the control tag 410 and where the base unit 110 is located outside a sterile field the package containing the handpiece 200 is opened only after entering the sterile field.

In one embodiment, referring to FIGS. 6 and 7, a device for treatment of tissue adjacent the dermal-hypodermal junction, includes a waveguide 220 having a distal end 224 configured to emit electromagnetic radiation and a proximal end 222 configured to receive electromagnetic radiation and a handle cap 400 having a connection end, e.g., a cap connector 420, adapted to connect to a handle 210, the handle cap 400 is coupled to the waveguide 220 and the proximal end 222 is substantially adjacent the connection end 420. In one embodiment, the device has a control tag 410 disposed in the handle cap 400. Optionally, the handle cap 400 has a tissue contact end adapted to contact tissue. The tissue contact end of the handle cap 400 can be substantially adjacent the waveguide distal end 224. In some embodiments, at least a portion of the handle cap 400 surrounds at least a portion of the external surface of the waveguide 220. The waveguide 220 can have a core 226 surrounded by a buffer 228 (see, e.g., FIG. 5). The waveguide 220 can be substantially rod shaped.

Referring now to FIGS. 1, 7, and 8A, in one embodiment, a control tag 410 is disposed in a handle cap 400 surrounding at least a portion of a waveguide 220. The handle cap 400 is connected to the handle 210 of the hand piece 200 by screwing the handle connector 420 threads into complementary threads in the handle 210. The user brings the control tag 410 of the hand piece 200 into proximity with a receiver located on, for example, the base unit 110 of the system 100, which activates the system 100 to enable EMR flow to the waveguide 220 of the hand piece 200. The EMR flow is activated to a limited amount of time (e.g., from about 30 minutes to about 8 hours, or about 4 hours) as dictated by the information stored in the control tag 410. Suitable control tag receivers or transponders may be located in, for example, a data board located in the cluster connector and/or the base unit of the system.

At least a portion of the hand piece 200 waveguide 220 is inserted by the practitioner into the body of a subject. In some embodiments, an incision is made in a subject's skin prior to insertion of the waveguide 220. In other embodiments, the distal end 224 or the tip 225 of the waveguide 220 makes the incision into the subject's skin. Once inserted, the waveguide 220 may be employed for excision, vaporization, ablation, and coagulation of soft tissue. Soft tissue includes, for example, skin, cutaneous tissue, subcutaneous tissue, striated and smooth tissue, fat, muscle, cartilage meniscus, mucous membrane, lymph vessels and nodes, organs and glands. The system and/or the hand piece 200 may be employed for laser assisted lipolysis.

In some embodiments, energy is released via the distal end 224 tip 225 of the waveguide 220. For example, during treatment of a subject, treatment energy is released into the body of a subject via the distal tip 225 of the waveguide 220. In one embodiment, a control tag 410 is employed and energy may be released into the body of a subject via the waveguide 220 for a limited number of hours, e.g., for about three hours. Upon lapse of the discrete quantity of time, the waveguide 220 becomes inactive. To continue with treatment, the user removes the waveguide 220 together with the handle cap 400, insert a replacement waveguide 220 and handle cap 400 into the handle 210, and then activate the replacement hand piece 200 by bringing the control tag 410 of the hand piece 200 into proximity with a receiver located on, for example, the base unit 110 of the system 100 to enable EMR flow to the waveguide 220 of the hand piece 200.

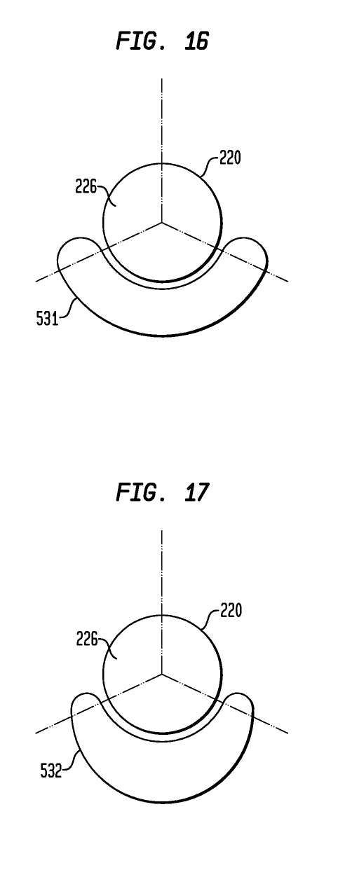

The hand piece 200 can be employed to treat soft tissue in the dermal-hypodermal region. The power density of the EMR emitted at the distal end of the waveguide 220 will be selected to suit the desired treatment. In one embodiment, where the waveguide 220 is employed to treat soft tissue including localized areas of fat and/or dermal tissue, the waveguide distributes a power density ranging from about 25 W/cm² to about 1 MW/cm² to the soft tissue being treated. The size and relative stiffness and/or strength of the waveguide 220 will be selected to suit the treatment area. For example, the waveguide 220 has a diameter ranging from about 200 μm to about 2 mm and a length that ranges from about 0.5 inch to about 12 inches and the ratio of the length to the diameter ranges from about 3 to about 150, or from about 50 to about 100. In one embodiment, the waveguide 220 has a diameter that ranges from about 0.800 mm to about 2 mm. In an embodiment where the waveguide 220 is employed to treat a relatively small area, such as the area under a patient's chin, a relatively smaller diameter e.g., about 0.95 mm, waveguide 220 may be selected. In one embodiment, the waveguide 220 has a Young's modulus that ranges from about 20 GPa to about 300 GPa, from about 65 GPa to about 150 GPa, or about 71 GPa. The waveguide 220 can have any of a number of cross sectional shapes including, for example, a circle, a rectangle, or an oval. The cross section of the waveguide 220 may be solid or, optionally, hollow (e.g., an annulus having any of a number of shapes including a circle, an oval, or a rectangle). The ratio of the length to the diameter and/or the Young's modulus and/or the waveguide cross sectional shape will be selected to provide a stiffness that enables the waveguide 220 to safely translate and rotate within the soft tissue being treated while avoiding damage to the waveguide 220, avoiding loss of waveguide function, and/or avoiding safety risk to the patient. In some embodiments, the waveguide 220 is self supporting and requires no additional support (see, e.g., FIGS. 16-19). In addition, the ratio of the length to the diameter and/or the Young's modulus may be selected to enable the tip 225 of the waveguide 220 to pierce through the patient's skin, for example.

Referring now to FIGS. 1, 6 and 7, the waveguide 220 has a proximal end 222 configured to receive electromagnetic radiation and a distal end 224 configured to emit a first optical radiation wavelength range suitable for liquefying adipose tissue and a second optical radiation wavelength range suitable for treating dermis tissue. In one embodiment, the waveguide 220 is capable of emitting multiple wavelength ranges. In another embodiment, a first wavelength range is suitable for liquefying adipose tissue and a second wavelength range is suitable for treating dermis tissue. Optionally, a first optical radiation wavelength is generated prior to a second optical radiation wavelength.

Once the waveguide 220 of the hand piece 200 is inserted into soft tissue adjacent the dermal-hypodermal junction the soft tissue is irradiated with at least one wavelength range. In one embodiment, the waveguide irradiates the soft tissue with a first wavelength range and with a second wavelength range. In one embodiment, the first wavelength range is generated prior to the second wavelength range. Optionally, at least one of the first wavelength and the second wavelength is an optical radiation wavelength. In one embodiment, the first wavelength range is suitable for liquefying adipose tissue (e.g., from about 910 nm to about 930 nm) and the second wavelength range is suitable for treating dermis tissue (e.g., from about 970 nm to about 990 nm). Optionally, where the first wavelength range is suitable for liquefying adipose tissue the liquefied adipose tissue is removed by, for example, palpating the liquefied fat to exit through the location of waveguide 220 insertion or suctioning liquefied adipose tissue with an aspiration device. In one embodiment, the second wavelength range is suitable for coagulating dermis tissue and/or for heating dermis tissue. Optionally, at least one of the first wavelength range and the second wavelength range tightens skin tissue.

In another embodiment, the first wavelength range is suitable for liquefying adipose tissue (e.g., from about 910 nm to about 930 nm) and the second wavelength range is suitable for ablating tissue (e.g., from about 1900 nm to about 11000 nm, or from about 2700 nm to about 3000 nm, or about 2940 nm). Optionally, where the first wavelength range is suitable for liquefying adipose tissue, all or a portion of the liquefied adipose tissue is removed via one or more holes created by ablating the patient's skin (e.g., the dermis and the epidermis). The one or more holes provide paths by which the liquefied adipose tissue exits the patient's body via the holes made through the patient's skin. In some embodiments, the second wavelength is selected to push out a the skin (e.g., a portion of dermis and a portion of epidermis) thereby creating a hole; a portion of the liquefied adipose tissue is likewise pushed out concurrently and/or subsequently via the path created by the one or more holes. Thus, the second wavelength pushes the tissue material to the external surface of the patient's body from inside the patient's body. In some embodiments, an additional force is applied to the liquefied adipose tissue to aid in removal of the liquefied adipose tissue from the patient's body via holes created from inside the body. Any of a number of means (e.g., palpating the liquefied fat to exit through the ablation hole(s), external suction force, a pushing force) may be selected to apply such an additional force for liquefied adipose tissue removal via the one or more ablation hole(s). In some embodiments, all or a portion of the liquefied adipose tissue is also removed by, for example, palpating the liquefied fat to exit through the location of waveguide 220 insertion and/or suctioning liquefied adipose tissue with an aspiration device via the location of waveguide 220 insertion.

Optionally, the second wavelength range is applied such that the second wavelength irradiates, coagulates, and/or ablates a portion of the tissue area and/or tissue volume being treated. Such methods and devices have become known as fractional technology. With fractional technology, the damage (e.g., heating, coagulation, and/or ablation) occurs within smaller sub-volumes or islets within the larger volume being treated. The tissue surrounding the islets is spared from the damage. The neighboring healthy tissue that surrounds the islets expedite a thorough healing process that occurs faster than where a larger volume of damaged tissue is present.

In one embodiment, the second wavelength is suitable for ablation and pushes out a portion of the skin (e.g., a portion of dermis and a portion of epidermis) to create two or more sub-volumes or islets within the larger volume being treated. At least a portion of the liquefied adipose tissue is likewise pushed out concurrently and/or subsequently via one or more of the fractional islets. In some embodiments, a portion of the liquefied adipose tissue exits via the location of waveguide 220 insertion by palpating or by suction with an aspiration device. Ablative holes may be employed to push out liquefied adipose tissue from any of a variety of treatment areas (e.g., small areas such as the chin, areas such as under the upper arms, larger areas such as the upper thigh) that were treated with a waveguide 220.

Where fractional ablative holes are formed through the skin (e.g., the dermis and epidermis) from inside the subject's body there is an opportunity for tightening, retracting, and/or shrinking the dermis and/or the epidermis through the skin tissue recovery. Fractional ablation of the skin (e.g., the dermal and epidermal tissue) from inside the subject's body can promote skin rejuvenation to improve the skin tone, improve the skin texture, tighten the skin, improve the appearance of wrinkles in the skin, at least partially remove one or more wrinkles in the skin, remove all or a portion of pigmented lesions (e.g., melasma, port wine stains), and/or remove tattoos. In some embodiments, suction and/or vacuum is applied to an external surface of the patient's body such that matter pushed out via the ablation holes (e.g., vapor, liquefied adipose tissue, dermis particles, epidermis particles, dermis particles, pigmented lesion particles, tattoo particles, and/or fat tissue) is captured for disposal. Optionally, liquefied adipose tissue is removed through the fractional ablative holes formed through the skin. In other embodiments, fractional treatment from inside a subject's body is a singular treatment that does not involve melting adipose tissue.

Optionally, a portion of the fatty acid of the liquefied adipose tissue that exits the body via the ablative holes remains in the dermis and/or the epidermis where it acts as filler in the fractional pattern. Use of a subject's own liquefied adipose tissue as dermal and/or epidermal filler reduces and/or avoids a separate step of providing a dermal filler such as, for example, Restyline™. In addition, the use of a subject's own liquefied adipose tissue as a dermal and/or epidermal filler reduces and/or avoids risks of reaction (e.g., allergic reaction) to a separate (e.g., synthetic) filler. Thus, a subject's own liquefied adipose tissue can provide a natural filler.

In another embodiment, the second wavelength is suitable for heating and/or coagulation of tissue. The dermis is coagulated in a fractional pattern in which sub-volumes or islets of dermis are coagulated within a larger volume being treated. The healthy tissue that surrounds the treated sub-volumes expedites healing. In this way, the larger volume is treated. Fractional heating and/or coagulation may be employed to provide an appearance of and/or to achieve skin smoothness and/or skin retraction. Fractional heating and/or coagulation may be employed to treat dermis and/or epidermis tissue from inside the patient's body. For example, fractional coagulation can be employed to treat pigmented lesions (e.g., port wine stains, birth marks and melasma), vascular lesions (e.g., blood vessels), scars (e.g., striae, acne scars, traumatic scars and post surgical scars) and/or tattoos from inside the subject's body. Optionally, fractional treatment from inside a subject's body is a singular treatment that does not involve melting adipose tissue.

In one embodiment, referring now the FIG. 8B a waveguide 220 is shown in use inside a subject's body 1000. FIG. 8B shows a side firing tip 225 of the waveguide 220, however, any number of waveguide styles may be employed to create ablative holes. The waveguide 220 distal end 224 enters the body 1000 at the point of insertion 1001 and traverses through the epidermis 1010 the dermis 1008 and into a region of fatty tissue 1006. The distal end 224 has a tip 225 that is tapered at an angle α that ranges from about 30° to about 70°, or from about 45° to about 68°, or from about 55° to about 60°, or about 53°.

A method for treatment of tissue with a photocosmetic device includes: inserting a treatment device such as the waveguide 220 into a soft tissue adjacent the dermal-hypodermal junction, (i.e., the fatty tissue 1006 adjacent the dermal tissue 1008) irradiating the soft tissue with a first wavelength range suitable for liquefying adipose tissue (e.g., from about 910 nm to about 930 nm), and irradiating the soft tissue with a second wavelength range suitable for ablating tissue (e.g., from about 1900 nm to about 11000 nm, or from about 2700 nm to about 3000 nm). In one embodiment, the first wavelength range is generated prior to the second wavelength range. A depiction of transmission of radiation 1012 in the irradiation step is shown in FIG. 8B.

In one embodiment, where the first wavelength range is suitable for liquefying adipose tissue, all or a portion of the liquefied adipose tissue is removed via one or more holes created by ablating the patient's dermis and epidermis. At least a portion of the liquefied adipose tissue exits the patient's body via one or more ablated holes. In some embodiments, the second wavelength is selected to push out a portion of dermis and a portion of epidermis thereby creating a hole; a portion of the liquefied adipose tissue is likewise pushed out concurrently and/or subsequently via the path created by the one or more holes. In some embodiments, at least some water and proteins are pushed out concurrently and/or subsequently via the path created by the one or more holes. Thus, referring still to FIG. 8B, the second wavelength pushes the tissue material 1011 to the external surface of the patient's body 1004 from inside the patient's body 1002. In some embodiments, an additional force (i.e., suction, vacuum, palpation) is applied to the liquefied adipose tissue to aid in removal of the tissue (i.e., the liquefied adipose tissue and portions of epidermis, dermis, and other tissues such as vascular lesions and/or pigmented lesions) from the patient's body via holes created from inside the body.

In some embodiments, wherein a first end of the one or more hole ablated in the subject's tissue is on the inside surface of the subject's body and a second end of the one or more hole is on the inside surface of the subject's body at a distance below the external surface of the subject's body. For example, the second end of the one or more hole is from about 0.1 mm to about 1 mm below the external surface of the subject's body. In such embodiments, the hole may also be called a cavity in that it does not penetrate through both the dermis and/or epidermis of the subject's tissue.

Where ablation of the dermal and epidermal tissue is desired, suitable wavelength(s) (e.g., from about 1900 nm to about 11000 nm, or from about 2700 nm to about 3000 nm), and EMR beam fluence are selected to extend through the entire depth of the tissue (e.g., through the dermis and the epidermis). In one embodiment, where a second wavelength ablates a portion of the tissue area and/or tissue volume being treated thereby defining ablated cone volumes 1014 that aid in removal of liquefied adipose tissue from the patients body 1000, the cross section of the ablated volume is largest at 1015a, in the region that faces inside the patients body 1002 (i.e., the largest diameter of the cone faces the inside of the patient's body and ranges from about 5 mm to about 10 microns in diameter) and the cross section of the ablated volume decreases at 1015b toward the external surface of the patient's body 1004 (i.e., the epidermis and ranges from about 1 micron to about 250 microns). Thus, the diameter of the ablated cone decreases toward the external surface of the patient's body 1004, i.e., the diameter 1015b is less than the diameter 1015a.

The method of treatment of tissue with the photocosmetic device can also include a third wavelength range suitable for coagulation of blood vessels. The third wavelength range can be from about 400 nm to about 700 nm. The third wavelength range includes 630 nm, for example. In one embodiment, the method of treatment of tissue employs a third wavelength range in the visible light range. The wavelength range in the visible light range can be employed as an aiming beam to assist the practitioner conducting the method of treatment.

FIG. 8C shows a portion of a subject's body 1000 in which ablated holes 1014a, 1014b, 1014c, 1014d, 1014e, 1014g have been formed over a region of tissue. In accordance with a treatment method employing the photocosmetic device, the device is moved in the x direction forming ablative holes 1014. Optionally, the pulsing of the ablative wavelength radiation is synchronized with the movement of the photocosmetic device (e.g., the waveguide) such that the ablative holes formed in the subject's tissue are substantially controlled. Synchronization can be achieved by, for example, control of the motion of the device Controlling the motion of the device (e.g., the speed of device movement) can be accomplished by employing a drive mechanism that acts upon the device to translate the device in a desired direction at a controlled speed. Controlling the motion of the device (e.g., the speed of device movement) can also be achieved by feeding a signal (e.g., audible, visual, and/or tactile) to the practitioner such that practitioner moves the device at substantially consistent speed (e.g., within a desired speed range). Synchronization can be achieved by, for example, measurement of the actual motion of the device and altering the repetition rate of firing as a function of the measured actual motion to control the spacing between adjacent ablative holes. The actual motion of the device may be measured by any of a number of means including, for example, employing an accelerometer on the device to measure the rate, employing a position sensor located relative to the subject to measure actual position of the device as a function of time, or employing a speed sensor (e.g., employing the optical mouse principle) that measures the actual velocity of the device. The measured actual motion of the device can be processed by a controller to alter the repetition rate of firing as a function of the measurement, in this way, the ablative hole distance is substantially controlled.

The distance 1016 (l) between adjacent ablative holes (i.e., 1014a and 1014b) equals the speed (ν) of device movement (as dictated by, for example, the speed of movement by the practitioner) divided by the repetition rate (v), which is the repetition rate of the firing. Accordingly, monitoring the rate and/or speed of photocosmetic device movement enables the repetition rate of the ablative holes being formed to be controlled. Likewise, the repetition rate of wavelengths for coagulation and/or heating of tissue from inside a patient's body can also be controlled because the distance (l) between adjacent treatment islets equals the speed (ν) of device movement divided by the repetition rate (v).

Referring now to FIG. 8B, side firing has certain benefits including that it more selectively hits its target and according can avoid treating tissue(s) where treatment is unwanted. For example, it is possible to treat skin (e.g., dermis 1008 and/or epidermis 1010) from an internal portion of the subject's body 1000. In one embodiment, a side firing waveguide 220 applies at least one wavelength range of radiation 1012 to heat and/or coagulate dermal tissue 1008 and/or epidermal tissue 1010. The waveguide 220 can be positioned in fatty tissue 1006 in the internal portion 1002 of the subject's body 1000. Once positioned inside the patient's body 1000, the waveguide 220 beam can face in an upward direction 1016 such that radiation treats the dermis 1008 and/or the epidermis 1010 through the dermis 1008. A wavelength range suited to treatment of the dermis 1008 and/or the epidermis 1010 is from 400 nm to about 3000 nm, from about 900 nm to about 3000 nm, from about 960 nm to about 2700 nm.

Additionally, the side firing waveguide 220 (e.g., as shown in FIG. 8D) may be employed to treat lesions and/or veins located within the dermis 1008 and/or the epidermis 1010. The radiation treatment may be conducted (e.g., to treat leg veins) by coagulation of veins at suitable wavelengths such as 980 nm and 1440 nm, for example. Suitable wavelength for vein treatment may range from about 600 nm to about 1500 nm, for example. Non-ablative frequencies may be employed to treat conditions such as blood vessels, veins, and lesions (e.g., port wine stains) from the inside and suitable wavelength ranges include from about 400 nm to about 3000 nm, or about 400 nm to about 1200 nm, or about 400 to about 600.

The pathway holes or islets formed through the dermis and the epidermis by ablation via the second wavelength may be any or a number of shapes. The ablation hole shape(s) may be determined by the ability to control the EMR beams within the tissue. In addition, the ablation hole shape(s) may be controlled by employing micro optics in the tip of the waveguide, for example.

Thus, depending upon the wavelength(s), temporal characteristics (e.g., continuous versus pulsed delivery), and fluence of the EMR; the geometry, incidence and focusing of the EMR beam; and the index of refraction, absorption coefficient, scattering coefficient, anisotropy factor (the mean cosine of the scattering angle), and the configuration of the tissue layers; and the presence or absence of exogenous chromophores and other substances, the holes and/or the islets can be variously-shaped volumes extending through the skin (e.g., from the dermal-hypodermal junction through the dermis and through the epidermis).

If the beams are not convergent, such beams will define one or more volume (e.g., hole(s) or islets) of substantially constant cross-sectional areas in the plane orthogonal to the beam axis (e.g., cylinders, rectanguloids). Alternatively, the beams can be convergent, defining volumes of decreasing cross-sectional area in the plane orthogonal to the central axis of the beams (e.g., cones, pyramids). The cross-sectional areas can be regular in shape (e.g., ellipses, polygons) or can be arbitrary in shape.

Where ablation of the dermal and epidermal tissue is desired, suitable wavelength(s) (e.g., from about 1900 nm to about 11000 nm, or from about 2700 nm to about 3000 nm), and EMR beam fluence are selected to extend through the entire depth of the tissue (e.g., through the dermis and the epidermis). In one embodiment, where a second wavelength ablates a portion of the tissue area and/or tissue volume being treated thereby defining ablated cone volumes that aid in removal of liquefied adipose tissue from the patients body, the cross section of the ablated volume is largest in the region that faces inside the patients body (i.e., the largest diameter of the cone faces the inside of the patient's body and ranges from about 5 mm to about 10 microns in diameter) and the cross section of the ablated volume decreases toward the exterior surface (i.e., the epidermis and ranges from about 1 micron to about 250 microns) of the patients body (i.e., the diameter of the cone decreases toward the exterior surface of the patients body).

Where the second wavelength is applied fractionally, generally, though not necessarily, the lattice is a periodic structure of islets, and can be arranged in one, two, or three dimensions. For instance, a two-dimensional (2D) lattice is periodic in two dimensions and translation invariant or non-periodic in the third. For example, and without limitation, there can be layer, square, hexagonal or rectangle lattices. The lattice dimensionality can be different from that of an individual islet. A single row of equally spaced cylinders is an example of a 1D lattice of 3D islets. For certain applications, an “inverted” lattice can be employed, in which islets of intact tissue are separated by areas of EMR-treated tissue and the treatment area is a continuous cluster of treated tissue with non treated islands. Each of the treated volumes can extend from a first depth to a second depth. The orientation of the lines for the islets in a given application need not all be the same, and some of the lines may, for example, be at right angles to other lines. Lines also can be oriented around a treatment target for greater efficacy. For example, the lines can be perpendicular to a vessel or parallel to a wrinkle. Many islet configurations are possible, such as cylinders, spheres, ellipsoids, cubes or rectanguloids of selected thickness. The shapes of the islets are determined by the combined optical parameters of the beam, including beam size, amplitude and phase distribution, the duration of application and, to a lesser extent, the wavelength.

The parameters for obtaining a particular hole and/or islet shape can be determined empirically with only routine experimentation.

By suitable control of wavelength, focusing, incident beam size at the tissue and other parameters, the hole(s) and/or islets, regardless of shape, can extend through the tissue volume. Where the second wavelength is fractional, most configurations of a lattice of islets can be formed either serially or simultaneously. Islets in the same or varying depths can be created, and the islets at varying depths can be either spatially separated or overlapping. Optionally, some islets can be ablative and others non ablative (i.e., coagulation and/or heating).

The geometry of the void(s) and/or islets affects the thermal damage in the treatment region. Since a sphere provides the greatest gradient, and is thus the most spatially confined, it provides the most localized biological damage, and can therefore be preferred for applications where this is desirable. Other geometries that increase the surface to volume ratio of the void(s) and/or islets may be preferred for other applications.

The size of the individual islets within the lattices of EMR-treated islets can be varied. For example, one or more individual islet within the lattice of EMR-treated islets can be sized relatively large compared to other individual islets. In one embodiment, where the relatively large sized islet is an ablative islet that penetrates from inside the subjects body from the dermis through the epidermis the relatively large sized islet can simplify the exit of liquefied adipose tissue. The healing of damaged tissues is more effective with smaller damage islets, for which the ratio of the wound margin to volume is greater.

As a general matter, the size of the EMR-treated islets can range from 1 μm to maximum length of targeted tissue in any particular dimension. For example, and without limitation, a lattice of substantially linear islets can consist of parallel islets having a length of approximately 300 mm and a width of approximately 10 μm to 3 mm to treat the length of a blood vessel. As another example, and without limitation, for substantially cylindrical islets in which the axis of the cylinder is orthogonal to the tissue surface, the depth can be approximately 10 μm to 4 mm and the diameter can be approximately 10 μm to 1 mm. For substantially spherical or ellipsoidal islets, the diameter or major axis can be, for example, and without limitation, approximately 10 μm to 1 mm. Thus, in some embodiments, the islets can be used to treat a specific portion of the target tissue surrounding a region of injury or in other embodiments treat the entire target tissue so as to induce a generalized tissue response throughout the target.

When considering the size of the optical, thermal, damage or photochemical islets, it is important to note that the boundaries of the islets may not be clearly demarcated but, rather, may vary continuously or blend into the untreated tissue (or differently- or less-treated tissue). For example, EMR beams are subject to scattering in various tissues and, therefore, even beams of coherent light will become diffuse as they penetrate through multiple layers of cells or tissues. As a result, optical and photochemical islets typically may not have clear boundaries between treated and untreated volumes. For some parameters, the transition from treated to untreated tissue will be quick and the boundaries of the islet will be well defined. For other parameters, the transition will be more gradual and less well defined. Similarly, thermal islets typically will exhibit a temperature gradient from the center of the islet to its boundaries, and untreated tissue surrounding the islet also will exhibit a temperature gradient due to conduction of heat. Finally, damage islets can have irregular or indistinct boundaries due to partially damaged cells or structures or partially coagulated proteins. As used herein, therefore, the size of an islet within a lattice of islets, refers to the size of the islet as defined by the intended minimum or threshold amount of EMR energy delivered. This amount is expressed as the minimum fluence, Fₘᵢₙ, and is determined by the nature of the cosmetic or medical application, for example, for destroying tissue, Fₘᵢₙ can be determined by the minimum fluence necessary to ablate the tissue or vaporize water. In each case, the size of the EMR-treated islet is defined by the size of the tissue volume receiving the desired minimum fluence.

The size of a damage islet can be either smaller or larger than the size of the corresponding optical islet, but is generally larger as greater amounts of EMR energy are applied to the optical islet due to heat diffusion. For a minimum size islet at any particular depth in the tissue, the wavelength, beam size, convergence, energy and pulse width have to be optimized.

The EMR-treated islets can be located at varying points within the tissue in the dermal-hypodermal region. The desired depth of the islets depends upon tissue thickness (e.g., the thickness of the dermis and/or the epidermis). For creating deep damage islets, long pulse widths coupled with surface cooling can be particularly effective.

In a given lattice of EMR-treated islets, the percentage of tissue volume which is EMR-treated is referred to as the “fill factor.” The fill factor is defined by the volume of the islets with respect to a reference volume that contains all of the islets. The fill factor may be uniform for a periodic lattice of uniformly sized EMR-treated islets, or it may vary over the treatment area.

Generally, the fill factor can be decreased by increasing the center-to-center distance(s) of islets of fixed volume(s), and/or decreasing the volume(s) of islets of fixed center-to-center distance(s). Thus, the calculation of the fill factor will depend on volume of an EMR-treated islet as well as on the spacing between the islets.

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

The center-to-center spacing of islets is determined by a number of factors, including the size of the islets and the treatment being performed. Generally, it is desired that the spacing between adjacent islets be sufficient to protect the tissues and facilitate the healing of any damage thereto, while still permitting the desired therapeutic effect to be achieved. In general, the fill factor can vary in the range of 0.1-90%, with ranges of 0.1-1%, 1-10%, 10-30% and 30-50% for different applications. In the case of damage islets it can be important that the fill factor be sufficiently low to ensure that there is undamaged tissue around each of the damage islets sufficient to prevent bulk tissue damage and to permit the damaged volumes to heal. When the density and distribution of these is sufficient within the targeted region a generalized recruitment of healing throughout the region appears to be elicited. This has the advantage that repair is elicited without initial loss of function.

Application of EMR to form lattices of islets of tissue injury has the advantage of extending and recruiting the healing needed to more fully and completely restore function to the entire affected tissue. The EMR lattices preferably will be of sufficient density, depth and volume to stimulate cellular reactions throughout the adjacent and affected surrounding tissue, although treatments of less than the entire affected tissue or of lower potency are possible according to certain embodiments. Treatment of the surrounding affected target tissue as well as the affected tissue damaged in the process of access to the target tissue speeds recovery and function. In some embodiments, the EMR treatment islets may be microscopic in size. Additionally, in some applications, the EMR treatment islets may be formed at temperatures below those that produce coagulation or destruction. In still other cases, EMR treatment islets may be formed by ablating or desiccating tissue.

Embodiments described herein are capable performing a therapeutic treatment on internal tissue by concentrating applied radiation of at least one selected wavelength at a plurality of selected, three-dimensionally located, treatment portions, which treatment portions are within non-treatment portions. The specific wavelength, focal depth and intensity will be based upon the intended use. The differences in tissue properties, size, thickness and constituent properties will need to be considered. Size and shape of the waveguide may also be designed to reach and limit properly the zone of tissue to which the EMR is applied.

In one embodiment, the second wavelength range is suitable for coagulating dermis tissue and/or for heating dermis tissue. Optionally, at least one of the first wavelength range and the second wavelength range tightens skin tissue from inside the subject's body. Heating and/or coagulation of dermal tissue from inside the subjects body can provide an improved external appearance of the tissue.

Referring now to FIG. 8E, in one embodiment, a side firing waveguide 220 applies at least one wavelength range of radiation 1012 to treat the fat tissue 1006. The waveguide 220 can be positioned in the internal portion of the subject's body within the fatty tissue 1006. Once positioned inside the subject's body 1002, the waveguide 220 radiation beam 1012 can face in a direction 1018 away from the dermis 1008 and/or the epidermis 1010. For example, the side firing tip can be aimed in a downward direction 1018 or in a sideways direction (not shown) to treat the fatty tissue 1006. A wavelength range suited to treatment of fat tissue 1006 (e.g., a wavelength range that liquefies adipose tissue is from about 910 nm to about 940 nm, from about 920 nm to about 930 nm, or about 925 nm. Employing the side firing waveguide 220 in a direction 1018 away from the dermis 1008 and/or the epidermis 1010 enables treatment of the fatty tissue (e.g., liquefying adipose tissue) while protecting the dermis 1008 and/or the epidermis 1010 from radiation 1018. Such a technique can be employed where, for example, the practitioner seeks to avoid exposing the dermis 1008 and/or the epidermis 1010 to too much energy and/or heat. For example, in areas of a patient's body where the dermal layer is thin, delicate, was previously treated, or does not require heating and/or coagulation.

Referring now to FIG. 8F, inside a subject's body 1000, between muscle 1009 and dermis 1008 is connective tissue called fiber stents 1007. Fiber stents 1007 are bundles of connective tissue that are held between the dermis 1008 and the muscle 1009. As discussed here, fiber stents include soft tissue such as fibrous septa, which is composed of collagen fiber material similar to what is found in the dermis tissue. Fiber stents 1007 align and connect the muscle 1009 and the dermis 1008 to one another. The fiber stents 1007 traverse through at least a portion of fat tissue 1006 inside the subject's body 1002. In some subject's, generally in women, when a volume of fat tissue 1006 between stents 1007 (e.g., between one stent 1007a and another stent 1007b) is over a threshold amount it creates an uneven, dimpled, and/or bumpy appearance on the external portion of the body 1004 and these dimples 1003 and/or bumps in the tissue are recognized as cellulite. Cellulite is the interaction of the existing fat 1006 with the stents 1007. A person with low fat could have cellulite because they have tight stents 1007. In some instances, Cutting the stents 1007 in the region of the dimpled 1003 e.g., in the areas between the bumps with a knife to relieve the stress caused by the volume of fat tissue 1006 between stents 1007 (e.g., adjacent stents 1007a and 1007b) provides relief to the stress on the skin tissue that previously resulted in a dimpled and/or bumpy appearance. Cutting the stents 1007 can result in a flattening of the skin that was formerly bumpy in the region of the stent 1007. However, use of a knife inside the skin is dangerous because it risks unintended consequences including nerve damage and muscle damage, for example.

A side firing waveguide 220 may be employed to improve the appearance of cellulite. For example, the waveguide 220 tip can be shaped and adapted to be non-cutting. For example, the waveguide 220 tip can be adapted to traverse through the tissue inside the body 1002. When you adjust the tip such that it is next to the target fiber stent 1007 you switch on laser energy and do either ablation (cutting) of one or more of the stents 1007 to release the tension between the dermis 1008 and the muscle 1009. Alternatively, you can adjust the tip such that it is next to a target fiber stent 1007 and then coagulate the target stent 1007 to adjust the mechanical strength of the fiber stent 1007 to weaken it and give it more elasticity or to treat it in a way that it expands so that the stents 1007 do not pull the skin down to create the cellulite. In this way, the appearance of cellulite is diminished, though the fat 1006 is still present between the dermis 1008 and the muscle 1009. Any of a number of waveguide configurations may be employed to treat the stent 1007. In order to ablate or cut the stent 1007 a wavelength range of from about 400 nm to about 3000 nm, or from about 900 nm to about 3000 nm, or from about 910 nm to about 2700 nm, or from about 2600 nm to about 3000 nm, or about 2940 may be employed and a fluence of from about 0.001 Joule/cm² to about 300 Joule/cm², or from about 5 Joule/cm² to about 150 Joule/cm², or from about 10 Joule/cm² to about 40 Joule/cm² and a pulse width of from one femtosecond to about 10 milliseconds, or from about 10 microseconds to about 1 millisecond. In order to coagulate the stent 1007 a wavelength range of from about 400 nm to about 3000 nm, or from about 900 nm to about 3000 nm, or from about 910 nm to about 2700 nm, or from about 1300 nm to about 2700 nm, or from about 1410 to about 2100 and a fluence of from about 10 Joule/cm² to about 1000 Joule/cm², or from about 15 Joule/cm² to about 700 Joule/cm² and a pulse width of from one microsecond to continuous wave mode, or from about 1 millisecond to 5 seconds. A side firing waveguide 220 may offer better control relative to other waveguide configurations. For example, a side firing waveguide enables the waveguide to touch the stent 1007 before energy is applied to the stent 1007. The practitioner can be visually guided by the dimples 1003 on the subject's body 1000 to determine the region for treatment in an internal portion of the body 1002.

In one embodiment, a handpiece includes an aiming beam provides visual aid to assist treatment of the subject. For example, where cellulite is being treated, the waveguide 220 of the handpiece is inserted into the internal portion of the subject's body. The practitioner can line the aiming beam of the handpiece in line with a visible dimple indicative of cellulite present on the external position of the body 1004. Once aligned with the visible dimple, with the aid of the aiming beam, the waveguide 220 is positioned to treat the stent 1007 in the region of the visible dimple. The waveguide 220 can be employed to apply ablative energy to the stent 1007 and thereby release the tension between the muscle 1009 and the dermis 1008. Ablation of the stent 1007 can improve the appearance and/or eliminate the appearance of the dimple on the external position of the subject's body 1004. The waveguide 220 can also be employed to coagulate the stent 1007, which can relieve all or a portion of the tension in the stent 1007 thereby improving and/or eliminating the appearance of the dimple indicative of cellulite. After treatment, the aiming beam can be applied in the region of treatment to confirm that the stent 1007 in the region of the dimple was treated and that the appearance of the treated dimple is improved and/or has been eliminated.

In another embodiment, an aiming beam can be employed to locate a stent 1007 in a region of skin. The aiming beam can make visible to the practitioner the presence of the stent 1007 in the subject's body 100.

Where the waveguide 220 is employed by the practitioner to side fire the skin (e.g., the dermis and the epidermis) in parallel to the skin the aiming beam can be employed by the practitioner to control the treatment of the skin. More specifically, in real time during the treatment the aiming beam is used by the practitioner as a visual reference visible through the skin to indicate the intensity of the aiming beam on the dermis and/or the epidermis as the side firing waveguide is directed to the dermis and/or the epidermis from inside the subject's body. The intensity of the aiming beam on the skin surface reveals the intensity of the treatment to the tissue. For example, the practitioner can see from the viewpoint external to the subject's body, the attenuation of the aiming beam via the skin being treated. This provides visual feedback of the amount of treatment being delivered by the practitioner to the subject based on the attenuation (e.g., how diffuse or spread the pattern) of the aiming beam visible external to the subjects body. With this information, the practitioner can confirm that the desired treatment is being achieved and/or alter the treatment in light of the observed visual pattern.

Alternatively or in addition, a photodetector may be employed to likewise automatically detect the attenuation of the aiming beam light through the subject's skin. The photodetector can provide automatic feedback control to alter (e.g., lessen and/or increase) the energy delivery to the skin tissue through the dermis and the epidermis.

A waveguide can have a tip that is capable of side firing (e.g., firing along the lateral axis) and is capable of firing along the longitudinal axis. In one embodiment, a tip is employed that is capable of side firing, or effectively side firing, under only specific conditions. Optionally, absent those specific conditions that fires mostly along the longitudinal axis (e.g., any side firing is substantially inefficient and cannot have an appreciable effect on tissue). For example, when the waveguide is immersed e.g., in a certain medium the waveguide adapted to side fire fires substantially along the longitudinal axis. The tip may be separate from or integral with the waveguide. Referring now to FIG. 8G, optionally, a tip 225 is capable of effectively side firing (e.g., firing in a lateral direction) under only specific conditions and absent such conditions the tip 225 fires along its longitudinal axis. For example, one or more properties of the surrounding tissue and/or one or more properties of the waveguide 220 and/or the tip 225 determine whether or not a specific tip is capable of side firing. For example, the refractive index of certain types of tissue differs from one another. More specifically, tumescent solution has a refractive surface of about 1.33 skin (dermis and epidermis) has a refractive index of about 1.41, fibrotic tissue, connective tissue, and fiber stents have a refractive index of about 1.41, and fat has a refractive index of from about 1.47 to about 1.51. Referring again to FIG. 8G, in one embodiment, the tip 225 is capable of side firing when it is exposed to a relatively low refractive index (e.g., the refractive index of the stent 1007c, which is about 1.41. However, the same tip 225 fires along its longitudinal axis when exposed to a relatively high refractive index, the refractive index of fat tissue is from about 1.47 to about 1.51.

There is an angle α relative to the tip 225 that can determine how the waveguide 220 tip 225 will behave (i.e., how its light will behave) depending on the refractive index of the medium that the tip 225 angle α touches. For example, a tip angle α can be selected that will work well in one medium and not work well in another medium. For example, one angle α will behave well in dermis (e.g., predominately comprising water) and the same angle α will exhibit a different behavior in fat (e.g., predominately comprising lipids).

More specifically, a selected tip angle α will side fire in dermis but will fire substantially along the longitudinal axis in fat. This type of configuration could work well for creating ablative holes in the dermis and for treating adipose tissue along the longitudinal axis as is discussed with relation to FIG. 8B, for example.

In another embodiment, a selected tip angle α will side fire in dermis and will not fire in fat. In another embodiment, a selected tip angle α will side fire in fat and will not fire in dermis. In still another embodiment, a selected tip angle α will fire substantially along the longitudinal axis in fat and will side fire in dermis.

In one embodiment, controlling the relationship between the waveguide material (e.g., the fiber material selected), the selected tip angle α, and the numerical aperture of the fiber (as determined by the selection of core and cladding in the fiber) it is possible to achieve a variety of desired firing behaviors. For example, in one embodiment, a waveguide side fires when surrounded by a lipid medium (e.g., in adipose tissue) and the same waveguide fires substantially along the longitudinal axis when surrounded by a water medium (e.g., in dermis tissue). In another embodiment, a waveguide side fires when surrounded by a water medium (e.g., in dermis tissue) and the same waveguide fires substantially along the longitudinal axis when surrounded by a lipid medium (e.g., in adipose tissue). In one embodiment, the waveguide that has one firing direction in lipid tissue and another firing direction in water based tissue is made from, for example, silica and/or quartz. Generally, a waveguide and/or a waveguide tip made from sapphire will not change firing direction based upon the medium in which it fires, for example, an angled waveguide made from sapphire will side fire (with some leakage along other directions). The unchanged behavior of sapphire waveguides surrounded by different tissue mediums is due, in part, to the relatively high refractive index of sapphire about 1.74 relative to the refractive index of body tissues, which are not higher than about 1.51.

Referring again to FIG. 8G, the waveguide 220 and the tip 225, which is side firing in at least some tissue types, can be made from any of a number of materials. The waveguide 220 and the tip 225 may be integral or, alternatively, they may be separable from one another. For example, in one embodiment, the waveguide 220 and the tip 225 capable of side firing are both made from sapphire, suitable sapphire materials for use in such waveguide applications are known to the skilled person. Sapphire is a costly material; accordingly, the waveguide 220 may be fabricated from other materials such as quartz, optical glass, or ceramic material. The tip 225 may be replaceable such that it can be connected to the waveguide 220 by any of a number of suitable methods such as, tension fit, clip fit, screw fit, fusing, adhesive bonding and/or diffusion bonding. Accordingly, the tip 225 can be made of sapphire or garnet, for example, and can be connected to a waveguide 220 such that the tip 225 is aligned with the waveguide 220 and with the optical fiber of the handpiece. The waveguide 220 can be solid and/or hollow in the shape of, for example, an annulus.

A waveguide 220 with a tip 225 that can side fire may be used to treat any of conditions such as, for example, fat (e.g., lipid liberation), dermis (e.g., treatment of dermis conditions from inside the body of the subject), and/or fiber stents. Treatment of fiber stents can include ablating the stents (i.e., cutting of all or a portion of the stent) or relieving the tension on the fiber stents by, for example, tissue coagulation or weakening of the tissue via exposure to waveguide 220 energy (e.g., optical radiation).

In one embodiment, the light propagation through the waveguide 220 and/or through the tip is very controlled. The numerical aperture is indicative of the angular pattern of light that exits the waveguide 220 and/or the tip 225. In side firing applications it is desirable to have substantially controlled and/or substantially minimized divergence of the light beam. The numerical aperture depends on the refractive index of the cladding and the refractive index of the core in the waveguide 220 and/or the tip 225. Suitable core materials include quartz, sapphire, optical glass and/or optical ceramic. Suitable cladding materials include doped silica or polymer materials. The numerical aperture is a function of the difference between the refractive index of the core and the cladding of the waveguide 220 and/or the tip 225. The waveguide 220 and/or the tip 225 are made from a material having a numerical aperture that ranges from about 0.1 to about 0.6, or 0.15 to about 0.37, or from about 0.39 to about 0.52. The numerical aperture is equal to the sin of the half angle of the divergence (β). The divergence (β) of the light that exits the waveguide is related to, and substantially determinative of, the maximum angle at which the light exits the fiber. In addition, the medium being treated by the waveguide (e.g., dermis, stent, adipose tissue) has a refractive index, which impacts the type behavior of the waveguide 220 in the medium due at least in part to the angle (α) of the waveguide. The refractive index is indicative of the velocity by which light travels through the medium.

In one embodiment, referring to FIG. 8A-8G, at least a portion of the waveguide 220 (e.g., the waveguide tip 225) is activated or carbonized prior to being used in a method of treatment. Activating the waveguide enables the surface of the tip 225 to run at a higher temperature than a waveguide tip 225 in the absence of activation. More specifically, an activated waveguide tip 225 incorporates carbon or iron oxide (Fe₂O₃) runs at a higher temperature (e.g., on the order of several hundred degrees Centigrade higher) than a waveguide in the absence of activation. An activated tip 225 absorbs energy (e.g., laser light energy) and heats up. However both an activated waveguide tip 225 and a non activated waveguide tip 225 focus optical energy at a desired location (e.g., at a location beyond the waveguide tip 225). Because an activated (e.g., carbonized) tip 225 runs at a relatively high temperature it more effectively traverses tissue (e.g., in adipose tissue) than a non activated waveguide tip 225 that does not provide the high heating provided by an activated tip 225. In particular, at the start of its use the activated tip 225 moves more fluidly in tissue than the non activated tip 225.

Because an activated tip 225 runs at a relatively high temperature relative to a non activated tip 225, activating the waveguide 220 tip 225 enables feedback control of the waveguide tip 225. Thus, with such feedback control when the waveguide 220 tip 225 has temperature beyond a threshold amount the power level can be decreased and/or shut off to avoid excessive temperature exposure in the area of treatment. In this way, it is possible that damage to tissue may be avoided before it occurs. Where a feedback control mechanism measures the treated tissue of a subject (e.g., the external temperature of a subject's skin is measured for a temperature change in the underlying treated tissue) the temperature change delta is relatively small and difficult to measure. Measurement of a relatively small delta is relatively costly (e.g., requires costly equipment) and relatively difficult to accomplish. In addition, the tissue having been treated to a relatively high temperature risks damage to the subject having undergone at least some overtreatment. In contrast, the feedback of the temperature of the tip 225 can enable control of the energy (e.g., the temperature) at the tip 225 prior any or any significant or any substantial impact on the surrounding tissue, in this way overtreatment of all or a portion of tissue may be avoided.

The waveguide 220 tip 225 may be activated and/or carbonized by any of a number of means. Activation and/or carbonization are means of initiation of increased absorption of at least one wavelength (e.g., at or near an end such as the proximal end) of the waveguide 220 (e.g., the waveguide 220 tip 225) prior to use. In one embodiment, the practitioner activates the waveguide 220 tip 225 prior to use. The practitioner can contact the tip 225 with any of a number of activation and/or carbonization materials such as for example, Teflon, polymer, wood, metal, carbon, iron oxide, or combination or composite thereof. The practitioner activates the waveguide 220 tip 225 while in contact with the above referenced material at a suitable power level for a suitable period of time to activate and/or carbonize the tip. Suitable time conditions include a time of from about 0.01 seconds to about 5 seconds, or about 2 seconds. Suitable power conditions can include any power range available to the system 100, including, for example, a power level of 10 watts or less and the practitioner can set these settings and carry out the activation procedure. Optionally, the desired power level and/or time settings are pre programmed in the system 100 such that an “activation” button can store the suitable settings and the practitioner can press the “activation” button when activating the waveguide prior to use. It is desirable that the practitioner wear eye safety glasses to protect his eyes during the activation step. Alternatively, the tip 225 may be surrounded by a tip cap that includes one or more of the above described activation and/or carbonization materials; the tip cap protects the eyes of those in the presence of the activation step and can be removed prior to treatment of the subject with the waveguide 220. The tip cap can also protect the tip 225 from becoming misshapen during the activation step.

In another embodiment, the waveguide 220 tip 225 can be activated, for example, prior to packaging and/or sterilization of all or a portion of the hand piece 220. In one embodiment, the waveguide 220 tip 225 can be activated with another separate laser that is fired at the waveguide 220 tip 225. The waveguide 220 tip 225 can be in the presence of carbon particles when the laser is activated. The carbon particles can be incorporated into the tip 225 to thereby carbonize the tip 225.

In another embodiment, the waveguide 220 tip 225 is placed in contact with carbon and/or iron oxide (Fe₂O₃) and energy (e.g., laser light) travels through the waveguide 220 tip 225, which hits the carbon and/or iron oxide to change the phase of the tip 225 such that it incorporates particles into the tip 225. In this way, the tip 225 is activated.

The method of treatment of tissue with the photocosmetic device can also include a third wavelength range suitable for coagulation of blood vessels. In accordance with a method for treatment of veins, e.g., leg veins, a wavelength range suitable: for blood coagulation, to target hemoglobin, to target MET-hemoglobin, to target water in a blood vessel or to target water in a vein can include the wavelengths from about 400 nm to about 600 nm, from about 750 nm to about 1100 nm, or from about 1300 nm to about 2400 nm. In one embodiment, the third wavelength range is in the visible light range at, for example, from about 60 nm to about 700 nm, and this visible light wavelength range may be used by the practitioner employing the photocosmetic device for diagnostic visualization and for feedback control.

The waveguide 220 wavelengths can range from about 400 nm to about 3000 nm Suitable wavelength ranges can include wavelength bands around (+/−20 nm), 410 nm, 920 nm, 975 nm, 1060 nm, 1208 nm, 1440 nm, 1715 nm, and 180 nm to 2000 nm, 2050 nm to 2150 nm, or 2100-2320, for example. The waveguide power can range from about 0.1 W to about 100 W, or from about 5 W to about 60 W.

Optionally, the photocosmetic device can be used for photodynamic therapy treatment of skin organs with wavelengths of the device matched to the peak absorption of a photosensitizer applied to the area being treated.

In one embodiment, the hand piece 200 suitable for laser assisted lipolysis is employed to treat localized areas of undesired fat using a minimally invasive procedure. The hand piece 200 is used to remove a conservative amount of fat from the lower chin, upper arm or thigh region. This hand piece 200 uses selective photothermolysis to liquefy adipose tissue for subsequent removal by the practitioner. The hand piece 200 irradiates the adipose tissue at a wavelength range including 915 nm at a power level (e.g., 10 W) and contacts a region of tissue for a time period (e.g., about 1 millisecond to about 20 seconds) suited to the specific application. In some embodiments, the portion of the hand piece in contact with the soft tissue is in substantially continuous motion (e.g., moves in a fan like manner). In some embodiments, the diode bundle forms a 635 nm diode aiming beam that is delivered through the tip 225 via the optical fiber and waveguide 220. The 635 nm diode aiming beam provides cutaneous trans-illumination during treatment. The aiming beam aids the practitioner with hand piece 200 and more specifically waveguide 220 position during a laser assisted lipolysis treatment. The practitioner can use the aiming beam to aid in targeting the treatment area and in controlling the speed of waveguide 220 movements through the treatment area. All or a portion of any liquefied fat is removed from the body of the subject. In one embodiment, all or a portion of any liquefied fat is aspirated from the subject's body via a syringe through a 2 mm cannula. In another embodiment, all or a portion of any liquefied fat is aspirated from the subject's body using an aspiration pump. Other mechanisms can also be used, including applying exterior pressure to the treated area. In one embodiment, the waveguide 220 is hollow and has a circular cross sectional shape, optionally, the hollow annulus of the waveguide is employed as the aspiration tube through which melted and/or liquefied fat is removed from the treatment area.

Optionally, prior to, simultaneously with, or subsequent to adipose tissue liquefication the practitioner can treat all or a portion of the dermis in the dermal-hypodermal junction. In one embodiment, the practitioner selects a second wavelength range suitable for treating dermis tissue (e.g., from about 970 nm to about 990 nm). In one embodiment, the second wavelength range is suitable for coagulating dermis tissue and/or for heating dermis tissue. Optionally, at least one of the first wavelength range and the second wavelength range tightens skin tissue. In one embodiment, at least one of the first wavelength range and the second wavelength range can be provided in a fractional manner such that radiation is applied to internal tissue such that radiation is concentrated on a plurality of selected, three-dimensionally located, treatment portions, which treatment portions are within non-treatment portions. The skin tissue (i.e., the dermis and/or the epidermis) is treated with at least one of the first wavelength range and the second wavelength range to provide one or more of a skin tightening, a tightened appearance, skin retraction, a retracted appearance, improvement in the appearance of one or more lypoma, removal of one or more lypoma, improved appearance and/or removal of scars, pigmented lesions, vascular lesions, acne, tattoos, wrinkles, birthmarks and/or hair and improvement in skin texture such as reduced crepeiness. For example, where the radiation is applied in a fractional manner from inside the body of the subject as a result, the outward appearance of the skin is refreshed and has a more youthful appearance.

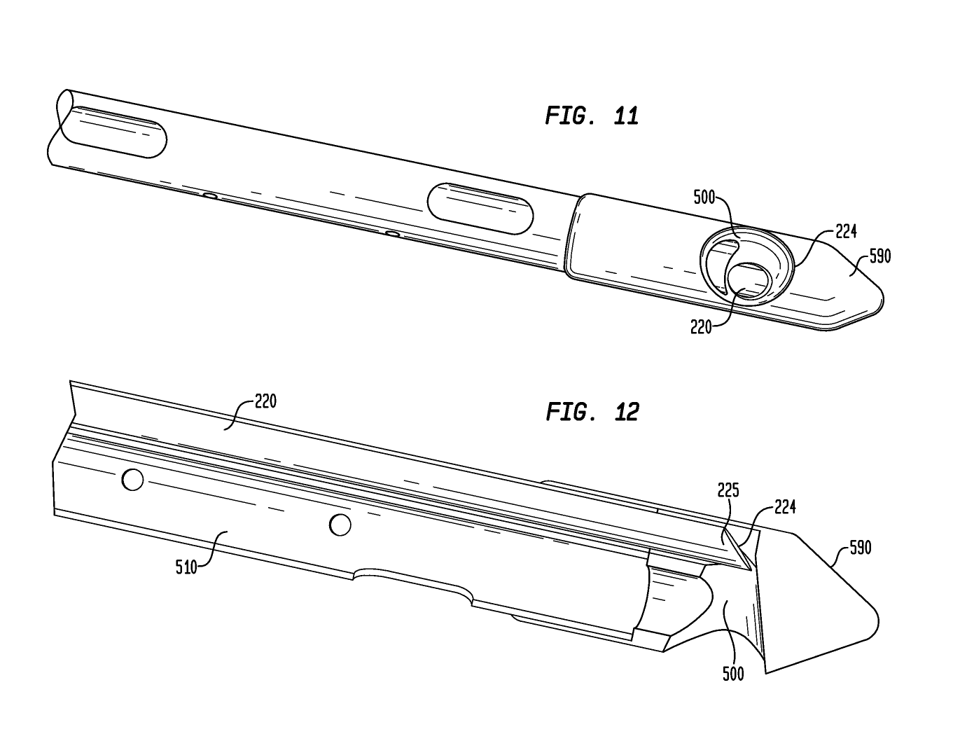

Referring now to FIGS. 11 and 12, in one embodiment, a hand piece includes a waveguide 220 combined with an aspiration tube 510. The waveguide 220 combined with an aspiration tube 510 can be disposed on one end of a handle. In one embodiment, a cover 590 is disposed at and surrounds the distal end 224 of the waveguide 220. The cover 590 can protect at least portion of the distal end 224 and/or at least a portion of the tip 225 of the waveguide. In one embodiment, the distal end 224 of the waveguide 220 is visible through an aspiration aperture 500. The distal end 224 and/or tip 225 of the waveguide 220 can be, for example, beveled or shaped. The shape of the tip 225 can be selected to achieve a desired optical energy focus. In operation, the waveguide 220 liquefies adipose tissue and the aspiration aperture 500 together with the aspiration tube 510 removes all or a portion of the liquefied fat from the treatment area in the body of the subject. In one embodiment, referring also to FIG. 3F, fat is liquefied and/or melted at a wavelength of 920 nm and to prevent heating of the nearby dermis by the liquefied and/or melted fat, the liquefied and/or melted fat is removed from the dermal-transdermal region to prevent an undesired thermal impact to the nearby dermis. The aspiration tube 510 can be sized to enable a desired flow rate of liquefied adipose tissue. For example, the aspiration tube can have a diameter that ranges from about 1 mm to about 6 mm. The waveguide 220 and the aspiration tube 510 can be integrated to be a single object (e.g., be surrounded by a cover, a sheath, or a cannula). In one embodiment, a waveguide 220 and an aspiration tube are surrounded by a cover having a diameter of about 1 mm. Alternatively, the waveguide 220 and the aspiration tube 510 can be separate pieces that are, for example, adjacent to one another. Optionally, a waveguide 220 and an aspiration tube 510 are bonded to one another by, for example, mechanical or chemical bonding methods.

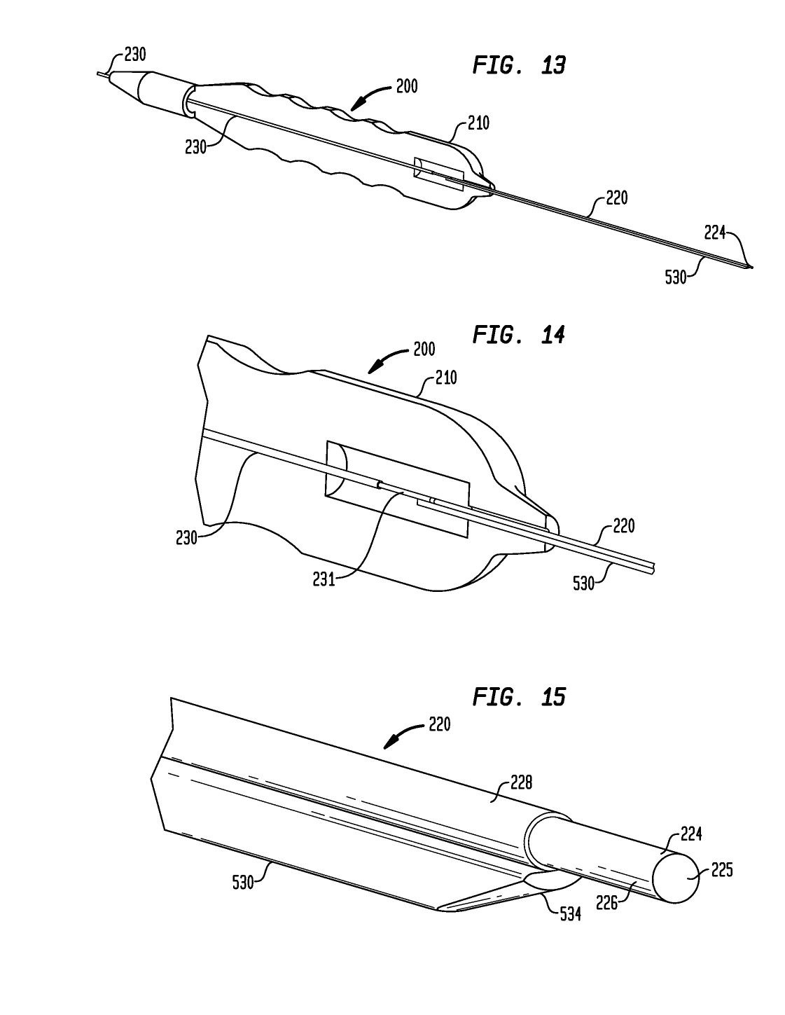

Referring now to FIGS. 13-15, in one embodiment, the hand piece 200 waveguide 220 has at least one support 530 adjacent at least a portion of the waveguide 220. The support 530 can be adjacent the exterior surface of the waveguide 220. The waveguide 220 combined with a support 530 can be disposed on one end of the handle 210. The support 530 can be a drawn wire fabricated from a biocompatible material such as, for example, stainless steel. The support 530 can be, for example, a drawn wire. The support 530 can include one or more of: a ceramic, a metal, a polymer, and a copolymer, for example.

The support 530 can be employed to supplement and/or increase the stiffness to the waveguide 220. In one embodiment, the support 530 has a Young's modulus greater than the Young's modulus of the waveguide 220. For example, the support 530 has a Young's modulus that ranges from about 10 GPa to about 500 GPa, or from about 190 GPa to about 210 GPa. In one embodiment, the Young's modulus of the composite formed by the waveguide 220 and the support 530 ranges from about 65 GPa to about 210 GPa, or about 150 GPa, for example. In one embodiment, the waveguide 220 has a longitudinal axis and the support 530 extends along at least a portion of the waveguide 220 longitudinal axis.

The support 530 can be bonded to the waveguide 220 by an adhesive such as, for example, a biocompatible adhesive and/or an adhesive that has a bond that cures when exposed to ultraviolet light. Suitable adhesive's that may be employed include Loctite® 3051 available from Henkel Corporation, Rocky Hill, Conn. Other suitable means to bond the support 530 to the waveguide 220 include ceramic cement, soldering, and abrasion bonding, for example. In one embodiment, an adhesive employed to bond the support 530 to the waveguide 229 fails upon exposure to sterilization conditions (e.g., autoclave or chemical washing), in this way reuse to the waveguide 220 is avoided and post treatment disposal of the waveguide 220 is ensured.

In one embodiment, referring to FIG. 15, the support 530 is a drawn wire that has a tapered end 534. The tapered end 534 enables ease of insertion into a subject's body and ease of translation through the subject's soft tissue. In one embodiment, the support 530 is adjacent to, and adhered to, a buffer 228 that surrounds a waveguide 220 core 226. In another embodiment, the support 530 is adjacent to, and adhered to, a waveguide 220 core 226. The waveguide 220 distal end 224 has a tip 225 that can be blunt or shaped, for example. Referring now to FIG. 16, the support 531 can have a uniform thickness and be sized and shaped to fit adjacent at least a portion of the waveguide 220, e.g., the core 226. Alternatively, referring now to FIG. 17, the support 532 can have an uneven thickness and be sized and shaped to fit adjacent at least a portion of the waveguide 220, e.g., the core 226. All or a portion of the support can have a substantially concave shape. Alternatively, all or a portion of the support can have a substantially flat shape.

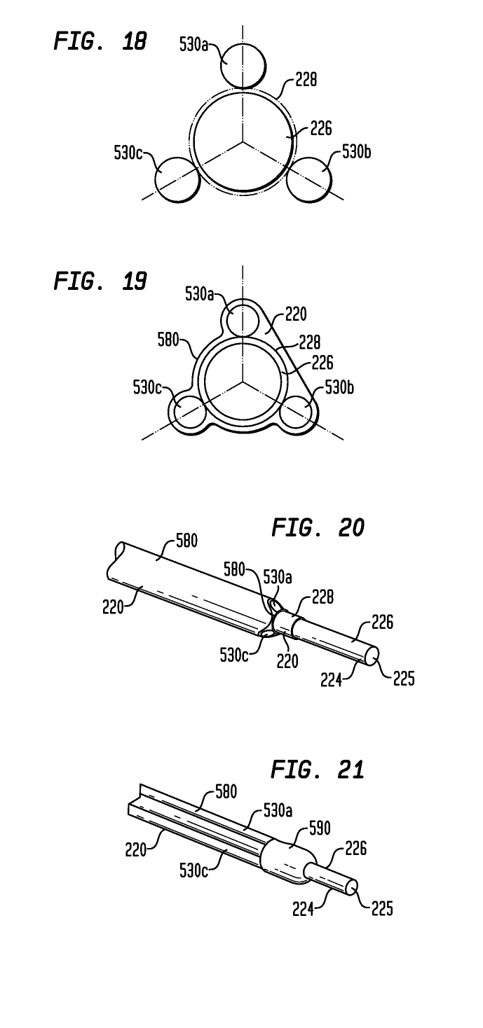

Referring now to FIGS. 18-20, in one embodiment, the waveguide 220 has a support 530a adjacent at least a portion of the exterior surface of the waveguide 220. For example, the waveguide 220 has a core 226 surrounded by a buffer 228 and at least one support (e.g., 530a) adjacent the exterior surface of the waveguide (e.g., adjacent the buffer 228). A coating 580 can surround at least a portion of the support 530a and at least a portion of the waveguide 220 and the coating binds the support 530a to the waveguide 220. Suitable coating 580 materials include, for example, nylon, Teflon® and polyimide.

Referring now to FIG. 20, in one embodiment, the waveguide 220 has multiple supports, for example, a first support 530a, a second support 530b, and a third support 530c are each adjacent at least a portion of an exterior surface of the waveguide 220. The waveguide 220 surrounded by one or more support(s) may be disposed at an end of the handle. A coating 580 surrounds at least a portion of each support 530a, 530b, and 530c and at least a portion of the waveguide 220 (e.g., the core 226 surrounded by the buffer 228). The coating 580 binds the first support 530a, the second support 530b, and the third support 530c to the waveguide 220. The supports can be employed to supplement and/or increase the stiffness to the waveguide 220. In one embodiment, the support 530a, the second support 530b, and the third support 530c combine to have a Young's modulus greater than the Young's modulus of the waveguide 220. For example, the support 530a, the second support 530b, and the third support 530c combine to have a Young's modulus that ranges from about 10 GPa to about 500 GPa, or from about 190 GPa to about 210 GPa. In one embodiment, the support 530a, the second support 530b, and the third support 530c combine to have an effective stiffness greater than the effective stiffness of the waveguide 220. For example, the support 530a, the second support 530b, and the third support 530c combine to have an effective stiffness that ranges from about 10 GPa to about 500 GPa, or from about 190 GPa to about 210 GPa. In one embodiment, the waveguide 220 has a longitudinal axis and the support 530a, the second support 530b, and the third support 530c each extend along at least a portion of the longitudinal axis.

Referring now to FIG. 19, the coating 580 can be applied to conform to the perimeter of the support (e.g., 530c) and the waveguide 220. Alternatively, the coating 580 can be applied in a manner that fills in curves and edges created by the support(s) and/or the waveguide 220 thereby creating, for example, flat surfaces, see, e.g., the flat surface between support 530a and the second support 530b. The coating 580 may be applied to provide a desired external surface to contact the patient's body when a portion of the hand piece is inserted in the patient's body.

Referring now to FIG. 21, a cover 590 may be placed over a region of the distal end 224. In one embodiment, the cover 590 has an aperture and when the cover 590 is placed over the distal end 224 of the waveguide 220 the tip 225 of the waveguide 220 passes through the aperture. As a result, when the cover 590 is place on the waveguide 220 the tip 225 of the waveguide 220 can contact the patient's soft tissue. The cover 590 can surround one or more region of the support(s) 530a, 530b, and 530c and/or the edge of the coating 580. In this way, the cover 590 provides a smooth or an even surface that contacts the patient's soft tissue. In one embodiment, the cover 590 is molded to provide a smooth transition from the tip 225 to the remaining portions of the waveguide 220. The cover 590 can be fabricated from a molded ceramic. A molded ceramic cover 590 can deflect heat.

Referring now to FIG. 22, a hand piece for the treatment of soft tissue from within a region of adipose tissue includes a waveguide 220 having a distal end 224 configured to emit electromagnetic radiation and a tube exteriorly surrounding the waveguide 220. A portion of the tube 620 extends beyond the distal end 224, and a cap 690 is disposed at an end of the tube 620. An antireflective surface and/or a reflective surface 691 is disposed on a surface of the cap 690 adjacent the distal end 224 and a transparent region is between the distal end and the reflective surface 691. In one embodiment, the tube 620 includes materials such as quartz, glass, sapphire, hard plastic, metal, or any combination thereof. In another embodiment, the tube 620 is coupled to at least a portion of the waveguide 220 by adhesive, tension fit, melting, or shrink fit, for example. The waveguide 220 surrounded by the tube 620 with a cap disposed at the end of the tube 620 may be disposed at an end of the handle.

Optionally, the cap 690 includes a heat sink. In one embodiment, at least a portion of the electromagnetic radiation emitted from the distal end 224 of the waveguide 220 is absorbed by the heat sink. A heated cap 690 provides lubrication to the soft tissue contacted by the cap 690. In another embodiment, at least a portion of the cap 690 is inserted into an open end of the tube 620.

The cap 690 can have a shaped surface 692 that is configured to contact the tissue of a subject or a patient. The shaped surface can have a curved, angled, parabolic, or other suitable shape. For example, the cap 690 can have a bullet shaped surface that is configured to contact a patient's tissue. In one embodiment, at least a portion of the electromagnetic radiation emitted from the distal end 224 of the waveguide 220 heats the shaped surface 692 of the cap 690. In another embodiment, at least a portion of the electromagnetic radiation emitted from the distal end 224 of the waveguide 220 refracts through the shaped surface 692. Refraction through the shaped surface 692 can have the refraction pattern 693, for example. At least a portion of the cap 690 (e.g., the shaped surface 692, the reflective surface 691) can have or be made from one or more of gold, copper, or a combination thereof.

In one embodiment, referring still to FIG. 22, at least a portion of the electromagnetic radiation emitted from distal end 224 of the waveguide 220 reflects off of the reflective surface 691 on the surface of the cap. The reflected emission can have the reflection pattern 694, for example. Optionally, the reflective surface 691 has a shape such as, for example, conical, concave, convex, angled, straight, multiple sided, single sided, curved, curved in multiple directions, or any combination thereof. In one embodiment, the reflective surface 691 provides a three dimensional distribution. The reflective surface 691 can have materials including gold, copper, mirror, multilayer dielectric, dielectric, or any combination thereof.

In one embodiment, the transparent region 697 between the distal end 224 and the reflective surface 691 is optically transparent. The transparent region 697 can include an adhesive gel. Alternatively, the transparent region 697 is a void space.

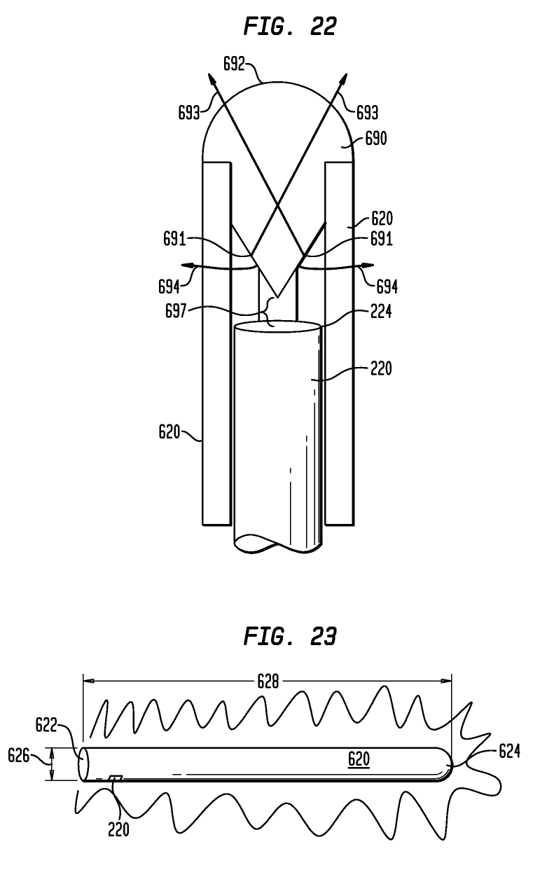

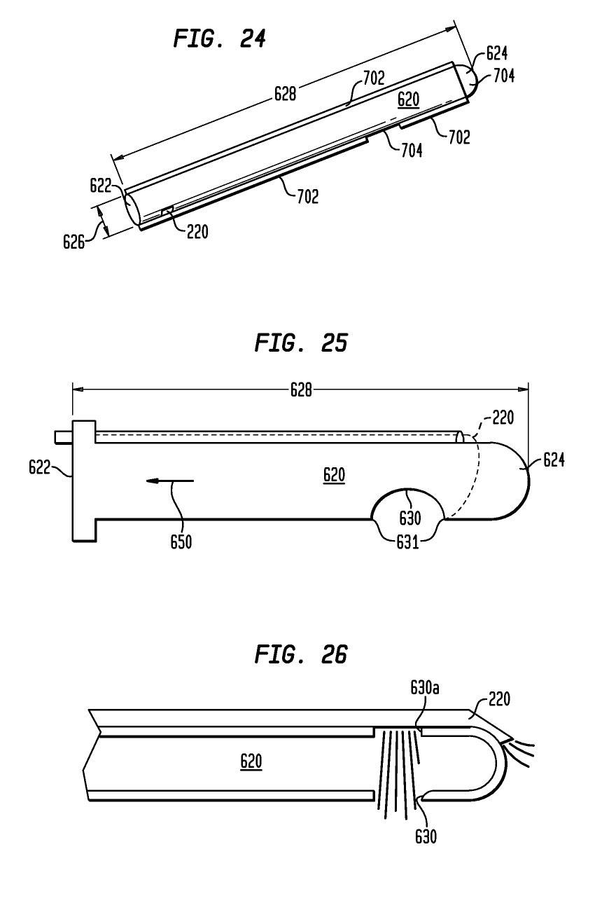

Referring now to FIG. 23, the hand piece for treatment of soft tissue can include a tube 620 having a width 626, a distal end 624, a proximal end 622, and a length 628 between the distal end 624 and the proximal end 622. The tube 620 may be made from, for example, quartz, glass, hard plastic, or metal. The proximal end 622 and the distal end 624 are capped and a waveguide 220, configured to emit electromagnetic energy, is disposed along the length 628 and at least a portion of the waveguide 220 is inside the tube 620. The tube 620 can be made of material including quartz, sapphire, glass, hard plastic, metal or a combination thereof. In one embodiment, a fluid (e.g., a gas such as air) is trapped inside the tube. In another embodiment, the tube 620 is configured to move the electromagnetic energy inside the tube 620 and along the length 628 and the width 626. The waveguide 220 can be a side firing fiber, for example. Optionally, the electromagnetic energy provides the working area. The waveguide 220 disposed in the tube 620 enables the electromagnetic energy to be broadly directed. In addition, the waveguide 220 disposed in the tube provides a relatively even distribution of optical energy to the treatment area. Optionally, referring now to FIG. 24, the tube 620 is selectively coated with insulative coating such that portions of the tube 620 are coated with insulation 702 and other portions of the tube 620 are free from insulation 704. In this way, the electromagnetic energy can be focused in the regions free from insulation 704 and less broadly directed then is described in association with FIG. 24. The insulative coating may be disposed on the inside surface of the tube 620, on the external surface of the tube 620, or a combination of both. Referring still to FIG. 24, in one embodiment, the tube 620 is coated with the insulative coating 702 and then portions of the tube 620 are etched to be substantially free from the insulative coating 704. The electromagnetic energy can be focused in the etched regions that are substantially free from insulative coating 704.

Referring now to FIG. 25, in one embodiment, the hand piece has a tube 620 having a distal end 624, a proximal end 622, and a length 628 between the distal end 624 and the proximal end 622. The distal end 624 is closed and an aperture 630 is disposed in the tube. The aperture 630 defines an edge. The edge can be a sharp edge 631. A waveguide 220 is disposed on an external surface of the tube 620 along at least a portion of the length 628. At least a portion of the waveguide 220 is located substantially opposite the aperture 630. In one embodiment, an end of the waveguide 220 is adjacent a portion of the aperture 630 edge.

Referring now to FIG. 27, in one embodiment, a waveguide 220 is disposed on an external surface of a tube 620. For example, the waveguide is disposed along at least a portion of the length 628. An aperture 630 is disposed in the tube 620. Optionally, one or more edges 631 of the aperture 630 are sharp. Optionally, suction 650 is imposed on the proximal end 622 of the tube. The suction 650 can be, for example, a vacuum force. In one embodiment, an end of the waveguide 220 is adjacent the aperture 630. The waveguide 220 can be position to melt the soft tissue exterior to the tube 620 so that the melted tissue can enter through the tube 620. When in contact with a soft tissue of a treatment area, the waveguide 220 heats up the soft tissue at a wavelength suitable for liquefying or melting adipose tissue. Alternatively, or in addition, the sharp edge 631 of the tube 620 cuts and/or shaves the soft tissue. In some embodiment, a wavelength suitable for blood coagulation is provided to coagulate blood during or after contact with the tube 620. In one embodiment, the waveguide 220 is paced at a wavelength of 715 nm and at a wattage level suited to the area of treatment. Any adipose tissue is melted and pulled through the tube 620 via suction 650 to exit the body of the subject. A portion of the waveguide 220 may be configured to emit a wavelength range suitable for dermis coagulation or for blood coagulation. In one embodiment, the waveguide 220 has a coating and the at least a portion of the waveguide 220 is etched to enable optical energy transmission along a portion of the tube 620 length 628. In one embodiment, the at least a portion of the aperture 630 is at an angle relative to a longitudinal axis 628 of the tube. In another embodiment, Referring now to FIGS. 28A and 28B, at least a portion of the tube 620 comprises an indentation 621 and at least a portion of the waveguide 220 is disposed in the indentation 621. The tube 620 may be sized to suction 650 and evacuate liquefied adipose tissue. The device can also include a vacuum configured to evacuate at least a portion of the tube 620. Optionally, the distal end 624 is closed by a cap.

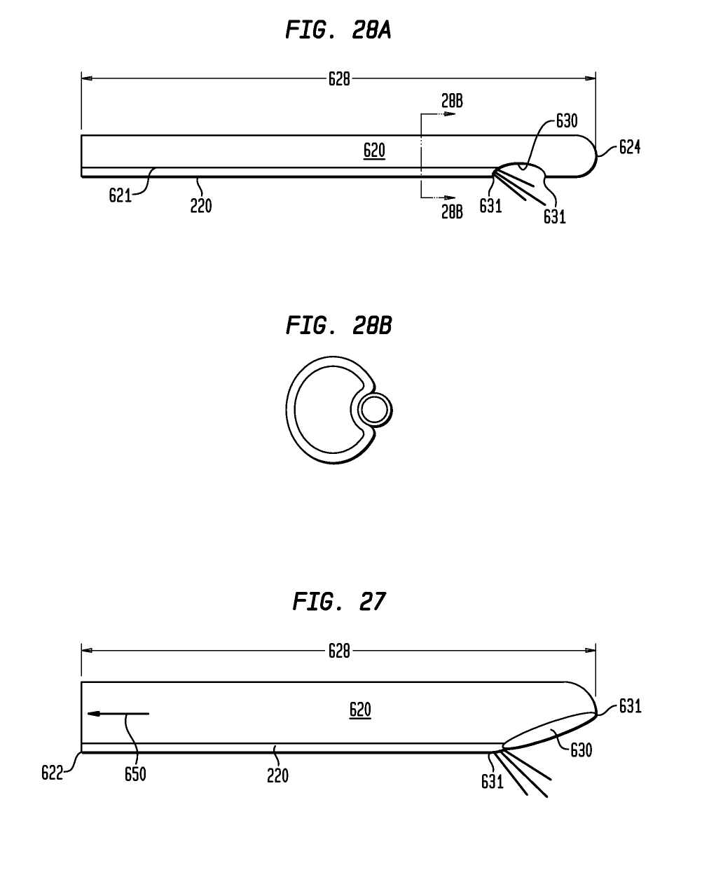

Referring now to FIG. 26, in one embodiment, the hand piece has a tube 620 having a distal end 624, a proximal end 622, and a length 628 between the distal end 624 and the proximal end 622. The distal end 624 is closed and an aperture 630 is disposed along the length 628 of the tube. A waveguide 220 is disposed along at least a portion of the length 628 and at least a portion of the waveguide 220 is located substantially opposite the aperture 630. An end portion of the waveguide 220 is configured to emit electromagnetic radiation and the at least a portion of the waveguide 220 is configured to emit electromagnetic radiation through the aperture 630. The waveguide 220 may be disposed on an external surface of the tube 620 along at least a portion of the length 628. In one embodiment, the tube 620 is sized to evacuate liquefied adipose tissue. In another embodiment, a vacuum is configured to evacuate at least a portion of the tube via suction 650. Referring still to FIG. 26, in one embodiment, at least a portion of the waveguide 220 is configured to emit a wavelength range suitable for liquefying adipose tissue. The end portion of the waveguide 220 may be configured to emit a wavelength range suitable for melting fat, dermis coagulation, or for blood coagulation. In one embodiment, the waveguide 220 has a coating (e.g., an insulative coating) and the at least a portion of the waveguide 220 is etched. Electromagnetic radiation may be transmitted via the etched portion through the aperture 630. In one embodiment, the portion of the waveguide 220 that transmits radiation through the aperture is adjacent an additional aperture 630a. The electromagnetic radiation transmitted through the aperture 630 may be within a wavelength range suitable to liquefy adipose tissue, coagulate dermis tissue, and/or coagulate blood. Optionally, the tube 620 has a coating and at least a portion of the tube 620 is etched. In some embodiments, the closed distal end 624 is a heat sink. In other embodiments, the closed distal end 624 is insulated. Optionally, at least a portion of the electromagnetic radiation emitted from the waveguide is absorbed by the closed distal end 624.

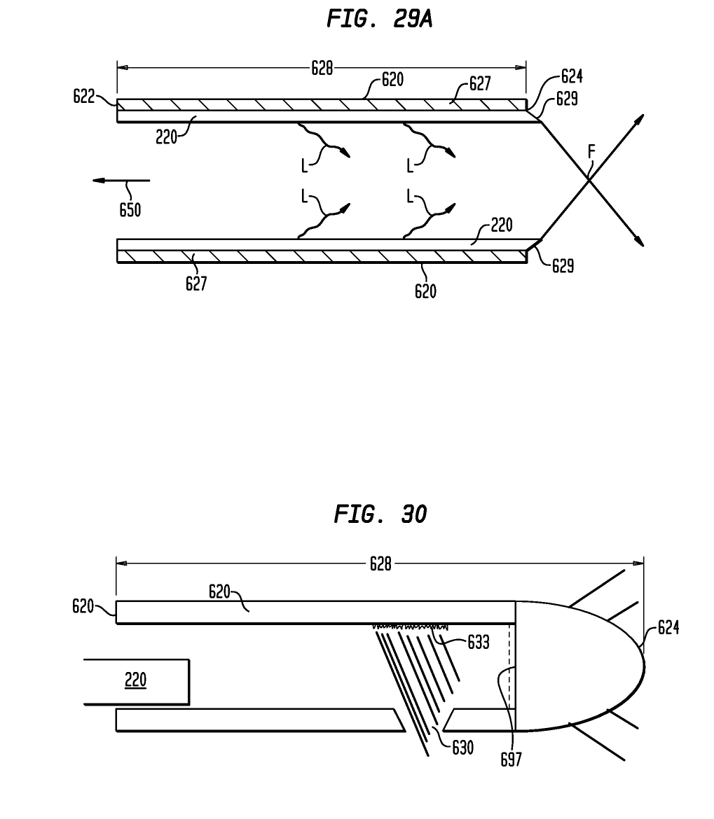

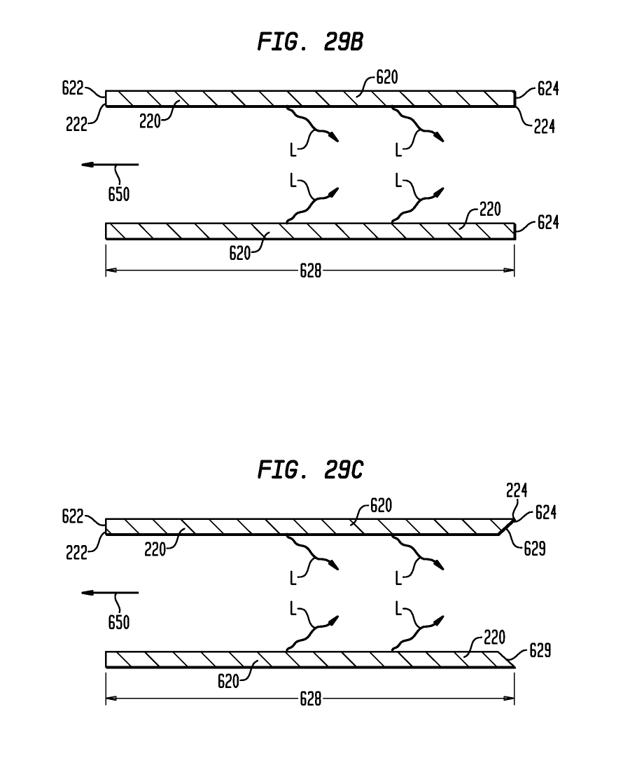

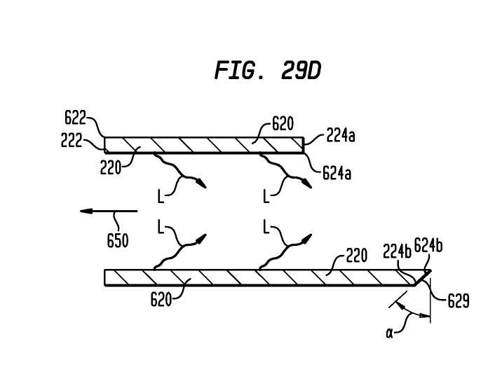

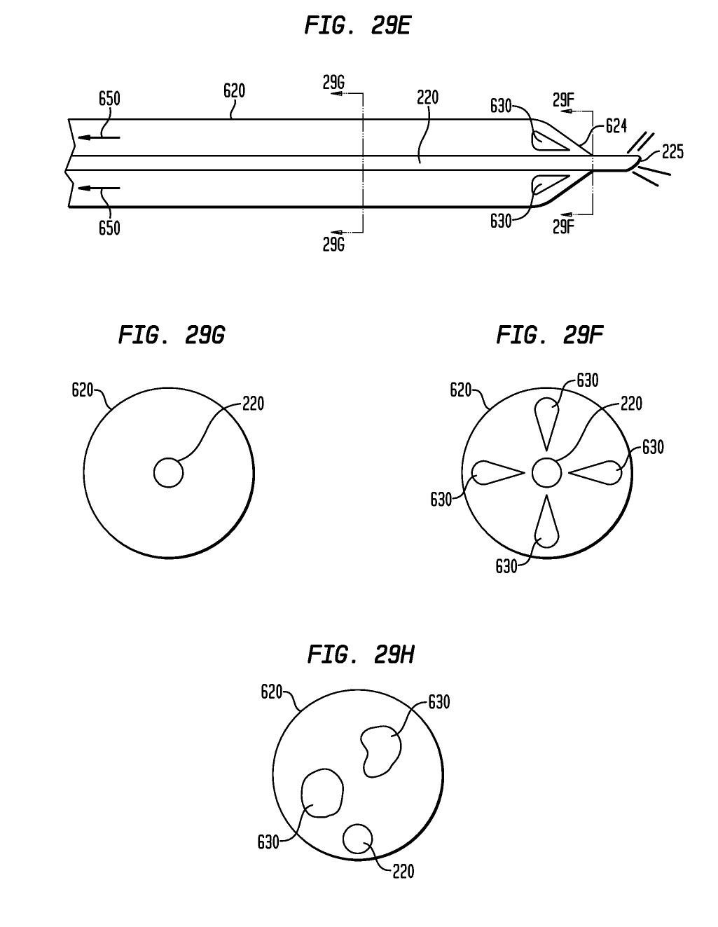

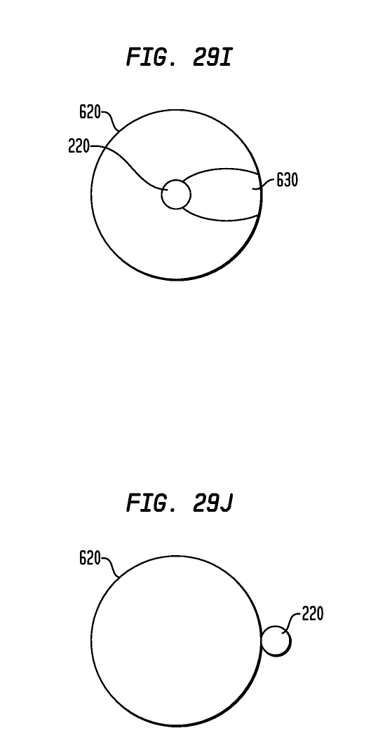

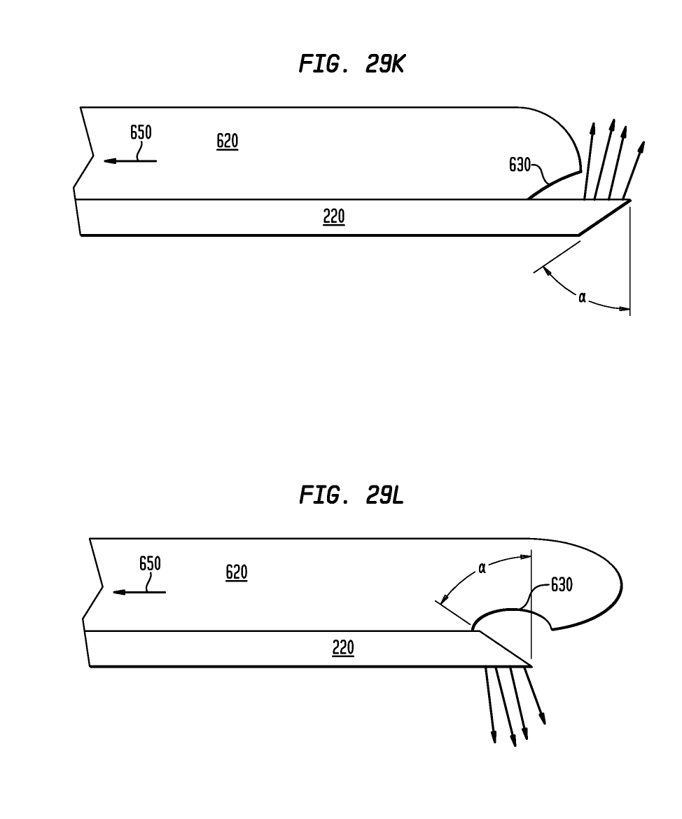

Referring now to FIG. 29A, in one embodiment, the hand piece has a tube 620 having a distal end 624, a proximal end 622, and a length 628 between the distal end and the proximal end. The distal end 624 is chamfered and a waveguide 220 is disposed in a portion of the tube. The waveguide 220 is configured to emit electromagnetic radiation along an inside surface of at least a portion of the tube 620. The waveguide 220 is configured to emit electromagnetic radiation that reflects off of the distal end 624. The photocosmetic device may also include a vacuum force that suctions 650 fluid from the distal end 624 toward the proximal end 622. The distal end 624 can have a beveled shape 629. In one embodiment, the proximal end 622 of the tube 620 also has a shaped and/or a sharpened edge. The tube 620 can be made from any of a number of materials including sapphire, quartz, glass, hard plastic, metal or a combination thereof. The tube 620 may comprise a support 627. The support 627 can be, for example, a metal support member adjacent a portion of the tube 620 or a metal support layer made surrounding at least a portion of the tube 620. The support 627 may be made from stainless steel, for example.

In one embodiment, the electromagnetic radiation focuses at the distal end 624, and optionally, at least a portion of the electromagnetic radiation converges at a focus point F adjacent the distal end 624 and external to the tube 620. The electromagnetic energy can irradiate and melt soft tissue that contacts focus point F. For example, at a wavelength of 915 nm the electromagnetic energy can melt adipose tissue. Optionally, the waveguide 220 provides internal leakage L along the inside surface of the tube 620. Internal leakage L can enable and/or promote flow of the melted tissue through the tube 620 with/or without a suction 650 force.

Referring now to FIGS. 29B and 29C, the hand piece waveguide 220 can form a tube 620 having a distal end 624, a proximal end 622, and a length 628 between the distal end and the proximal end. The waveguide 220 has a distal end 224 and proximal end 222. In one embodiment, shown in FIG. 29C, the distal end 624 is chamfered and has a beveled shape 627. Referring again to FIGS. 29B and 29C, the waveguide 220 is configured to emit electromagnetic radiation. The waveguide 220 is configured to emit electromagnetic radiation that reflects off of the distal end 224, 624. The photocosmetic device may also include a vacuum force that suctions 650 fluid from the distal end 224, 624 toward the proximal end 222, 622. The distal end 224, 624 can have a beveled shape 629. In one embodiment, the proximal end 222, 622 of the waveguide 220 tube 620 also has a shaped and/or a sharpened edge. The waveguide 220 tube 620 can be made from any of a number of materials including sapphire, quartz, glass, optical glass, optical ceramic, garnet or a composite thereof.

In one embodiment, the electromagnetic radiation focuses at the distal end 224, 624, and optionally, at least a portion of the electromagnetic radiation converges at a focus point adjacent the distal end 224, 624 and external to the waveguide 220 tube 620. The electromagnetic energy can irradiate, coagulate, ablate, and/or melt soft tissue. For example, where a wavelength range includes a wavelength of about 975 nm the electromagnetic energy can coagulate soft tissue such as skin tissue (e.g., dermis and/or epidermis) via the side firing waveguide 220 tube 620. In addition, simultaneous or subsequently a wavelength range that includes a wavelength of about 924 nm can be applied to melt adipose tissue to thereby provide lipid liberation. Optionally, the waveguide 220 provides internal leakage L along the inside surface of the tube 620. Internal leakage L can enable and/or promote flow of the melted tissue through the waveguide 220 tube 620 with/or without a suction 650 force.

Referring now to FIG. 29D, the waveguide 220 tube 620 can be side firing to enable side firing at least a portion of the energy being delivered. A side firing waveguide 220 tube 620 can have a beveled shape 629 at the waveguide 220 tube 620 distal end 224b, and 624b. As shown in FIG. 29D the distal end 224a and 624a is shorter than the distal end 224b and 624b. The distal end 224a and 624a can fire substantially along the longitudinal axis wherein the distal end 224b and 624b can fire substantially at an angle relative to the longitudinal axis (e.g., the distal end 224b and 624b can side fire),

The practitioner can move the waveguide 220 such that it faces the hypodermal junction when the wavelength range including 975 nm is applied. The practitioner can move the waveguide such that the wavelength range including 924 nm faces adipose tissue, e.g., in a direction away from the hypodermal junction. Alternatively, the side firing waveguide 220 tube 620 can be adapted, via choice of materials and the angle α such that the wavelength range including 924 nm has a different focus point than the focus point of the wavelength range including 975, more specifically the side firing waveguide 220 tube 620 travels substantially along the longitudinal axis to target adipose tissue for lipid liberation.

Optionally, the waveguide 220 provides internal leakage L along the inside surface of the tube 620. Internal leakage L can enable and/or promote flow of the melted tissue through the waveguide 220 tube 620 with/or without a suction 650 force.

Referring now to FIGS. 29A-29D, the waveguide 220 tube 620 can provide the functionality to a waveguide and a suction cannula previously requiring a waveguide separate from a suction cannula. Suitable waveguide 220 tubes 620 are sized to have an internal tube 620 diameter range from about 0.5 mm to about 6 mm, or from about 1 mm to about 5 mm, or from about 2 mm to about 3 mm. Suitable external tube 620 diameters range from about 1 mm to about 8 mm, from about 2 mm to about 7 mm, from about 3 mm to about 5 mm. The wall thickness of the waveguide 220 tube 620 ranges from about 0.5 mm to about 2 mm, or from about 1 mm to about 2 mm. The combination of the waveguide material selected, the diameter of the waveguide 220 tube 620, the wall thickness and/or and or cladding and/or buffer that is employed should be selected to enable the waveguide 220 tube 620 to substantially maintain its integrity during use in a surgical procedure. In some embodiment, the buffer and/or sheath are selected to act as a containment medium in the event that the core material breaks, cracks and/or shatters, in this way, contamination of the surgical site with foreign objects (e.g., a portion of the core material) may be avoided.

Optionally, in some embodiments, a waveguide 220 tube 620 may be made out of quartz with lower refractive index doping of the inner surface and/or the outer surface. The waveguide 220 tube 620 may be made from any of a number of optical materials (e.g., quartz, glass, optical ceramic, sapphire, or garnet) with a polymer coating having a refractive index that is less than the refractive index of the refractive index of the optical material, the polymer coating with the lower refractive index being disposed on the inner surface and/or the outer surface of the waveguide 220 tube 620.

In one embodiment, a bundle of separate waveguides may be fused together to form an annulus or a tube. The bundle of separate waveguides may be fused by heating, chemical, or any other means known to the skilled person. In addition, one or more of the separate waveguides that are fused to form an annulus or a tube may have distinct and/or different properties. In addition, one or more of the separate waveguides may be made of different materials. In this way, the separate waveguides that are fused to form a tube may be capable of selectively firing at, for example, different energy levels. The separate waveguides that are fused to form a tube may be capable of selectively firing under different conditions (e.g., some of the separate waveguides fire in one environment (e.g., fatty tissue) and others do not fire in that one environment (e.g., fatty tissue) but fire in another environment (e.g., tissue having a high water content).

In one embodiment, a waveguide 220 is employed together with a tube 620 and/or a cannula (or, optionally, the waveguide 220 and the tube 620 are one in the same as described in relation to FIGS. 29A-29D, for example) that enables the waveguide 220 to melt a volume X of adipose tissue (e.g., to liberate a volume of lipids to liberate) and enables the tube 620 and/or a cannula to evacuate a volume X, which is substantially the same as the volume X of adipose tissue that was melted. The volume of adipose tissue X that is has lipids liberated and substantially the same volume X of treated (e.g., melted and/or liberated) tissue is evacuated via aspiration at the same time or substantially the same time as the melting treatment is conducted. In one embodiment, the ability to evacuate the same volume that is melted simultaneously or substantially simultaneously enables the heated adipose tissue to be removed prior to or substantially prior to providing heat to surrounding tissue. By aspirating and/or evacuating the volume X simultaneous or substantially simultaneous with melting the volume X, uncontrolled heating by melted and/or liberated lipids via conduction, convection and/or radiation is minimized and/or avoided. In this way, safety and/or control associated with a laser lipolysis procedure is improved. Further, simultaneous or substantially simultaneous removal of the heated (e.g., melted and/or liberated lipid) tissue improves the speed of the procedure. For example, compared to a procedure where the adipose tissue is treated and then later the heated (e.g., melted and/or liberated) tissue is removed via aspiration, the control, safety, and/or speed of the procedure is improved.

The device includes a device for energy delivery (e.g., optical radiation, radio frequency, ultrasound energy, acoustic energy etc.) and a tube and/or cannula through which treated tissue (e.g., heated and/or melted and/or lipid liberated) tissue may be evacuated and/or aspirated. Energy is delivered to tissue (e.g., adipose tissue).

Formulae that govern this process provide:

P=V₁*Cv*ΔT*ρ

P is the power (e.g., optical radiation) delivered in Watts.
V₁ is the volume of liberated lipid produced per unit of time, which can also described as the rate of melting and/or lipid liberation m³/second.
Cv is heat capacity (e.g., the specific heat) of the fat Joules/kg/° C.
ΔT is the change in temperature in ° C.
ρ is the density of tissue to be removed in kg/m³.

The goal of the procedure is that V₁=Vₐ the rate of volume of liberated lipid V₁ is equal to the rate of volume of liberated lipid that is aspirated Vₐ. In order to ensure that V₁=Vₐ there must be communication between the energy delivery system and the aspiration (e.g., the suction) system. Thus, where V₁=Vₐ

Vₐ=P/(Cv*ΔT*ρ)

Balancing the V₁ and Vₐ may be accomplished by incorporating a feed back loop for example, by employing a flow meter to measure the volumetric rate of liberated lipid removal, the mass of aspirate may be measured as a function of time by employing a aspirate collection device together with a scale, and/or the volume of aspirate may be assessed as a function of time. Based on the information gathered via feedback the power can adjusted to maintain a desired ΔT.

To achieve the desired control, in one embodiment, the ΔT is kept to what is determined to be the lipid liberation temperature minus the normal physiologic temperature. In order to accomplish this control the amount of power delivered in Watts is controlled. Alternatively or in addition, the desired control of ΔT can be achieved achieve by controlling the Vₐ the rate of volume aspiration. The rate of volume aspiration can be controlled by, for example, the vacuum pump pressure and/or the stroking speed employed by the practitioner.

In order to control the rate of aspiration Vₐ such that it is substantially equal to the rate of lipid liberation V₁ there is an upper limit for both power (e.g., energy delivery) and suction (e.g., aspiration) that can be modulated.

In one embodiment, the amount of aspiration and the location of aspiration entry are controlled relative to the locus of the treatment of the adipose tissue (e.g., the melting and/or heating of the fat tissue). By locating the aspiration entry in proximity to the location of the newly melted tissue together with the level of aspiration regulated relative to the power level of the energy source it is more likely to achieve the removal via aspiration of solely the tissue that has been treated (e.g., melted and/or heated).

One problem with traditional suction assisted liposuction is that it exposes the tissue to an amount of mechanical stress that can destroy the tissue. When suction is used to assist laser lypolysis, the rate of suction can be such that in addition to removing at least a portion of the melted tissue (e.g., the lipid liberated tissue) the suction can mechanically remove tissue instead of or in addition to suctioning out the melted tissue. For example, in a two step procedure described herein, first a waveguide is employed to melt the adipose tissue, thereafter, the melted adipose tissue is aspirated by, for example, a suction assisted cannula; such procedures do not guarantee that the suction process removes only what's been liquefied, accordingly, some mechanical removal of tissue may also take place.

Treated adipose tissue (e.g., melted fat or liberated lipid tissue) is easier to evacuate with lower pressure than the same tissue that is not treated with energy to melt and/or liberate the lipid from the tissue. More specifically, melted fat tissue is less viscous than typical fat tissue (e.g., non melted fat tissue). In traditional suction assisted liposuction tissue is physically suctioned and pulling out of the subject's body. In the two step process of first exposing the fat tissue to the energy (e.g., lipid liberation) and second mechanically removing the treated fat tissue, some of the untreated tissue (e.g., non melted tissue) is mechanically removed.

In order to achieve the goal of removing via aspiration only or substantially all of the treated (e.g., melted and or lipid liberated) tissue and none of the untreated tissue, a number of configurations have been developed. In one embodiment, the amount of aspiration and the location of aspiration entry are controlled relative to the locus of the treatment of the adipose tissue (e.g., the melting and/or heating of the fat tissue). Alternatively or in addition, the tube and/or cannula is integrated with the energy such that the energy source produces energy that corresponds with aspiration such that the energy source and the aspiration (e.g., suction) are interdependent and adjust relative to one another. Alternatively or in addition, the energy (e.g., the power) is adjusted relative to the speed of movement of the device (e.g., the aspiration and/or suction) varies depending on the speed of the movement of the device (i.e., the device includes energy delivery (e.g., optical radiation, radio frequency, ultrasound energy etc.) and a tube and/or cannula through which treated tissue may be aspirated). Thus, in one embodiment, the energy varies relative the speed at which the practitioner moves the device and the aspiration rate likewise varies depending on the speed of the practitioners movements. Alternatively or in addition, the power can be adjusted depending on the speed and when the speed increases the power is increased; likewise when the speed decreases the power is decreased.

Referring now to FIGS. 29E-29G, the tube 620 and surround the waveguide 220 is a substantially concentric fashion. The distal end 624 of the tube 620 narrows and surrounds the waveguide 220 that exits through the distal end 624 of the tube 620. The tip 225 of the waveguide 220 is external to the tube 620. One or more apertures 630 are disposed substantially symmetrically through the shaped distal end of the tube. One or more of the apertures are in proximity to the waveguide 220 that exits the distal end 624 of the tube. When the device shown in FIG. 29F is in use inside a subject's body, the waveguide 220 tip 225 applies energy to adipose tissue. The waveguide 220 can apply a wavelength range that include 924, a wavelength that targets lipids. The applied wavelength melts adipose tissue (e.g., liberates lipids in the adipose tissue). In some embodiments, the melted adipose tissue is substantially liquefied. The device applies suction 650 that pulls the newly melted adipose tissue through the one or more apertures 630, the newly melted adipose tissue travels through the inside surface of the tube 620 external to the waveguide 220 housed therein to exit the device. In some embodiments, the melted adipose tissue contacts the waveguide 220 and is optionally delivered additional energy thereby as it exits the inside surface of the tube 620. By providing additional energy to the melted fat tissue as it journeys through the tube 620 via aspiration clogging of the tube 620 can be substantially avoided and/or improved. In other embodiments, the melted fat does not contact the waveguide 220 housed therein, because a barrier is present. The methods discussed herein (e.g., coordinating power, speed, and or aspiration) can be employed to control the device to enable only the melted fat to be aspirated and to prevent and/or avoid mechanical aspiration of non treated (e.g., non melted, non lipid liberated) fat tissue.

Other optional configurations of the waveguide 220 relative to the tube 620 are depicted at FIGS. 29H, 29I, and 29J and each Figure shows a different device from the same perspective as of the distal end of the tube 620 also shown in FIG. 29F. In FIG. 29H, the waveguide 220 is disposed on the inside of the tube 620 and is offset such that it is closer to the inside surface of the tube 620 than it is to the tube 620 center. Two apertures 630 are disposed in the end of the tube 620 and are placed in an asymmetric fashion relative to the waveguide 220. The waveguide 220 can optionally extend beyond the end of the tube 620, can be flush with the end of the tube 620, or can be recessed inside the end of the tube 620. In FIG. 29I, the waveguide is disposed on the inside of the tube 620 and is substantially centered in the tube 620. A single aperture 630 is disposed in the end of the tube 620 from about the outside surface of the waveguide 220 to about the inside surface of the tube 630. The waveguide 220 can optionally extend beyond the end of the tube 620, can be flush with the end of the tube 620, or can be recessed inside the end of the tube 620. In FIG. 29J, the waveguide 220 is disposed external to the tube 620 and is in physical contact with the external surface of the tube 620. In one embodiment, the distal end of the tube 620 is open. In another embodiment, the distal end of the tube 620 is tapered and closed and one or more apertures are disposed in the distal end of the tube 620 (not shown).

FIG. 29K shows the side of a tube 620 with a side firing waveguide 220 adjacent an external surface of the tube 620. The tube 620 has suction 650 capacities. During treatment inside a subjects body, the side firing waveguide 220 heats a path of tissue (e.g., can liberate lipid when a wavelength range including 924 nm is employed) and is followed in along the path of treatment by the tube 620. The tube 620 suctions 650 the newly melted fat for removal of the melted fat in real time (e.g., substantially simultaneously). The newly melted fat enters the tube 620 via one or more apertures 630 or optionally though an open distal end of the tube 620 (not shown).

FIG. 29L also shows the side of a tube 620 with a side firing waveguide 220 adjacent an external surface of the tube 620. The tube 620 has suction 650 capacities. However, in FIG. 29L the direction of the side firing waveguide 220 is reversed relative to the side firing waveguide 220 depicted in FIG. 29K. In FIG. 29L an aperture 630 is disposed in a side of the tube 620 along its longitudinal axis. The aperture 630 is in the region of the tissue treated by the side firing waveguide 220. During treatment inside a subjects body, the side firing waveguide 220 liberates lipid and is followed along the path by the tube 620. The tube 620 suctions 650 the newly liberated lipid through the aperture 630 (disposed in the region of the side firing waveguide treatment) along the longitudinal axis of the tube 620.

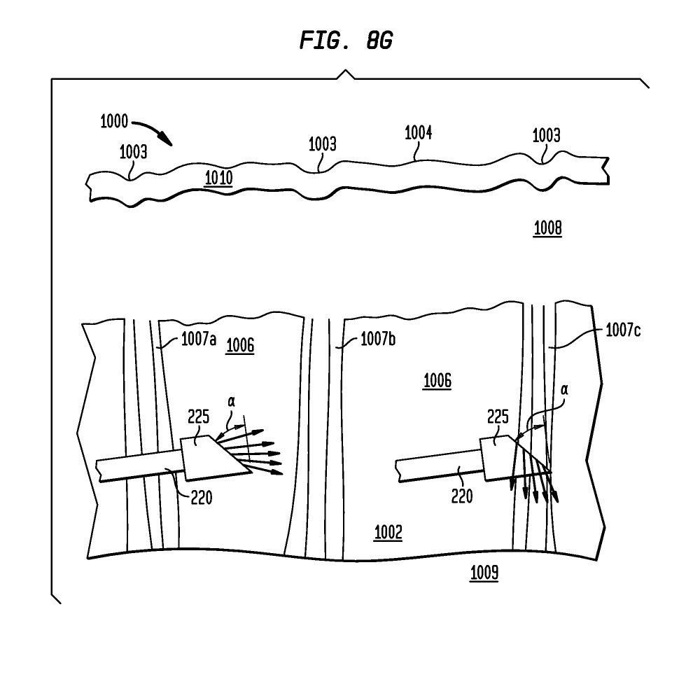

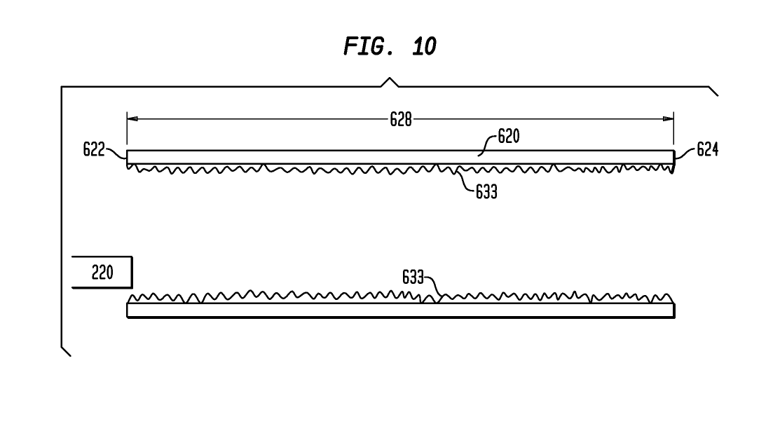

Referring to FIGS. 10, 22-27, 28A-28B, 29A-29L, and 30-32 the tube 620 can range is size from a diameter of from about 1 mm to about 8 mm, from about 2 mm to about 7 mm, from about 3 mm to about 5 mm and a length of from about 2 inches to about 12 inches, or from about 5 inches to about 10 inches.

Referring now to FIG. 10, in one embodiment, the hand piece has a tube 620 having a distal end 624, a proximal end 622, and a length 628 between the distal end 624 and the proximal end 622. All or a portion of the inside surface of the tube 620 has a reflective surface 633 and/or is made from or includes a material having a very low absorption. A waveguide 220 is disposed in at least a portion of the tube 620. Optionally, the waveguide is attached to the proximal end 622 of the tube 620. The waveguide 220 is configured to emit electromagnetic radiation, which travels along t least a portion of the inside surface of the tube 620. Optionally, the photocosmetic device includes a vacuum force that suctions fluid from the distal end 624 toward the proximal end 622. The distal end 624 and/or the proximal end 622 of the tube 620 may be shaped and/or have a sharpened edge. The tube 620 can be made from any of a number of materials including metal, sapphire, quartz, glass, hard plastic or a combination thereof.

In one embodiment, the distal end 624 of the tube 620 has an optical element such as, for example, a lens or sphere that can cover all or a portion of the otherwise open distal end 624.

Referring now to FIG. 30, in one embodiment, the hand piece has a tube 620 having a distal end 624, a proximal end 622, a length 628 between the distal end 624 and the proximal end 622, and the distal 624 end is closed. An aperture 630 is disposed along the length of the tube. A reflective surface 633 is disposed on an inside surface of the tube 620 on a side substantially opposite the aperture 630. Suitable materials that may be employed to provide the reflective surface include, for example, gold, copper, or other reflective material suitable for use in light and laser devices. A waveguide 220 is configured to emit electromagnetic energy and at least a portion of the waveguide 220 is disposed inside the tube 620. A portion of the electromagnetic energy can be transmitted through the aperture 630 to contact a patient's soft tissue. The hand piece can have a vacuum force configured to evacuate fluid from the distal end 624 toward the proximal end 622. Optionally, electromagnetic energy is provided to the tube 620 via the waveguide 220 at a wavelength suited to melting or liquefying adipose tissue. Sequentially or simultaneously the suction force 650 can remove the liquefied tissue from the treatment area via the inside surface of the tube 620.

The tube 620 can be made from any of a number of materials including sapphire, quartz, metal, hard plastic, glass or a combination thereof. In one embodiment, the closed distal end 624 is a heat sink that provides a heated surface that enables movement through soft tissue with the aid of lubrication provided by the heated closed end. In another embodiment, the closed distal end 624 is insulated. Optionally, at least a portion of the electromagnetic radiation emitted from the waveguide 220 is absorbed by the closed distal end 624. In one embodiment, a transparent region 697 is located adjacent the closed distal end 624 on the inside surface of the tube 620. The transparent region 697 can include an adhesive gel. Alternatively, the transparent region 697 can be a void space.

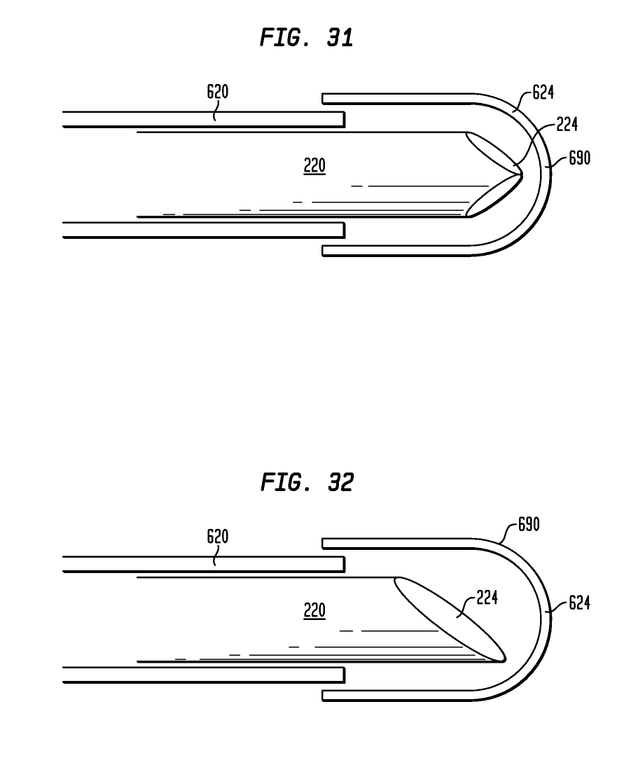

Referring now to FIGS. 31 and 32, in some embodiments, a hand piece includes at least a portion of a waveguide 220 comprising quartz, glass, and/or sapphire, for example, disposed inside a tube 620 having a closed distal end 624. Optionally, the distal end 624 of the tube is closed by a cap 690. Alternatively, the distal end 624 of the tube 620 has an integrated closed end. The tube 620 and/or the cap 690 may be made from any of a number of suitable materials including, for example, quartz, sapphire, metal, glass, or hard plastic.

The end 224 of the waveguide 220 disposed in the tube 620 is surrounded by a fluid, for example, a gas such as air. The end 224 of the waveguide 220 can be shaped to enable a desired electromagnetic transmission focus. Referring to FIG. 31, the end 224 of the waveguide 220 can be substantially triangular featuring two symmetrical facets. Optionally, an end of a waveguide 220 having symmetrical facets can be truncated to resemble a flat headed screw driver. In one embodiment, two or more facets can be asymmetrically located at the waveguide 220 end 224 such that the length of one facet is longer than the length of another facet and/or the angle where the two or more facets meet can be varied to achieve a desired electromagnetic transmission focus. In another embodiment, two or more facets are symmetrically located at the waveguide 220 tip and the tip end is truncated to provide a flat end.

Alternatively, referring to FIG. 32, the end 224 of the waveguide 220 can be substantially beveled. Optionally, all or a portion of the end 224 of the waveguide 220 can be coated or covered with a reflective surface such as, for example, a metal (e.g., gold or silver).

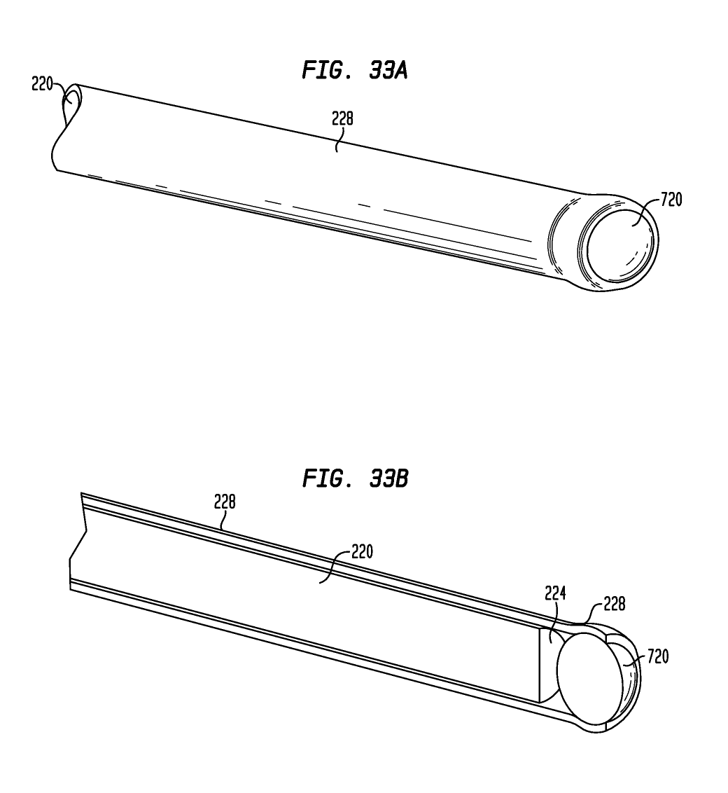

Referring now to FIGS. 33A and 33B, in some embodiments, a hand piece includes at least a portion of a waveguide 220 having a distal end 224 disposed adjacent an optical sphere 720. The at least a portion of the waveguide 220 and at least a portion of the optical sphere 720 are surrounded by a buffer 228. The waveguide 220 can be made from suitable materials including quartz, sapphire, or glass for example. The optical sphere can be made from any of a number of suitable materials employed in light based and laser based devices including, for example, quartz, glass, and/or sapphire. The buffer 228 can be a sheath or covering that is pulled over and/or surrounds at least a portion of the waveguide 220 and/or optical sphere 720. The buffer 228 can be made from any of a number of suitable materials including, for example, Polyetheretherketone (PEEK). In one embodiment, the optical sphere 720 physically contacts the end 224 of the waveguide 220. In another embodiment, the optical sphere 720 and the end 224 are adjacent, but are not in physical contact with one another. The end 224 of the waveguide can have a straight shape or, alternatively, is can be beveled or in a triangular shape as described with reference to FIGS. 31 and 32. The optical sphere 720 can focus energy emission that travels through the waveguide 220 from the hand piece. One benefit of the optical sphere 720 is that when placed adjacent the end 224 of the waveguide 220 and surrounded by the buffer 228 no additional alignment (e.g., no optical alignment) is required for the electromagnetic radiation to travel therethrough.

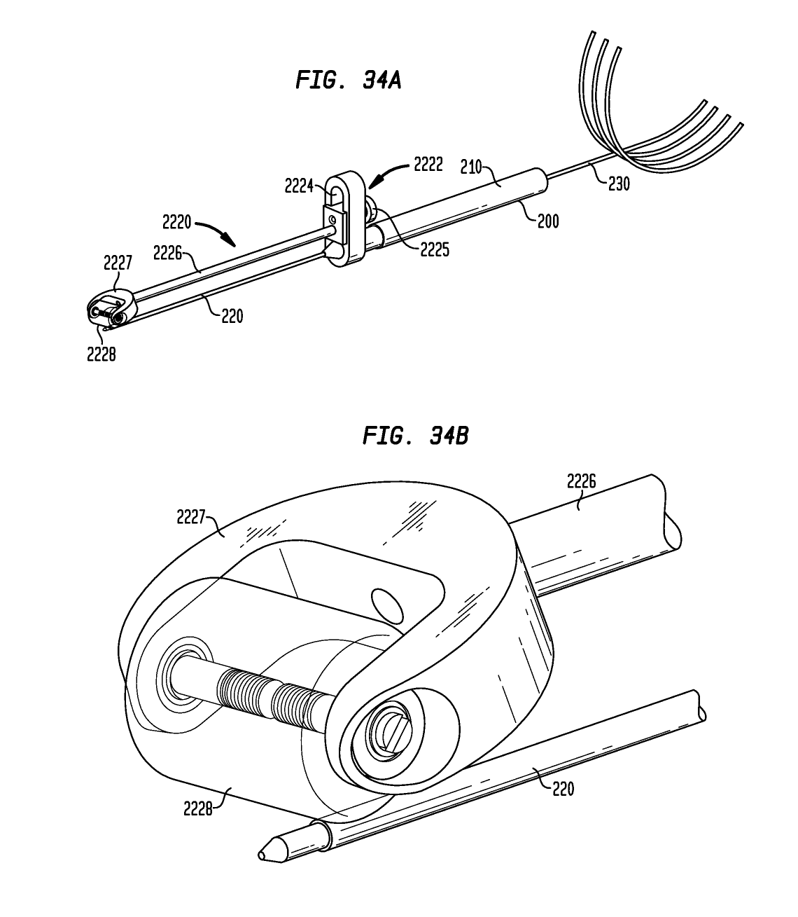

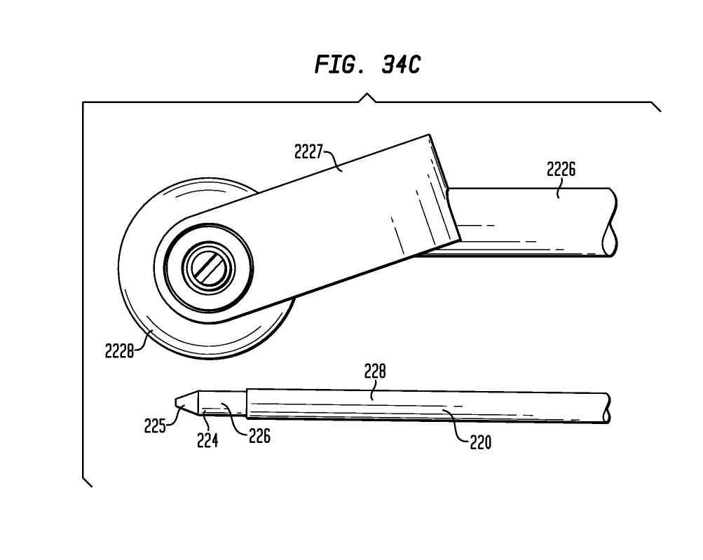

Referring now to FIGS. 34a, 34b, and 34c, a spacer 2220 can be employed to regulate and/or control the depth of the waveguide 220 inside the subject's body. More specifically, the hand piece 200 includes an optical fiber 230, a spacer 2220 and a waveguide 220. Referring now to FIG. 34a, the hand piece 200 includes a waveguide 220 and a spacer 2220 that together maintain a substantially constant distance there between.

When the hand piece 200 is in use to treat a subject, the practitioner inserts the waveguide 220 inside the body of the subject such that the waveguide 220 is located in an internal portion of the subject's body. All or a portion of the spacer 2220 is located on an external portion of the subject's body. A constant or substantially constant distance is created between the spacer 2220 and the waveguide 220. When in use the practitioner moves hand piece 200 substantially parallel to the subject's skin and at least a portion of the spacer 2220 contacts the external surface of the subject's skin. Thus, the hand piece 200 that creates a constant or substantially constant distance between the spacer 2220 and the waveguide 220 can control how the depth of treatment accessible to the waveguide 220. The controlled depth available with a hand piece 200 employing a spacer 2220 and a waveguide 220 can enable targeting of the desired treatment depth. Controlled depth can be aid in avoiding undesirable waveguide 220 treatment targets such as, for example, muscle, certain organ(s), and bone.

Referring again to FIG. 34a, the hand piece 200 has a waveguide 220 and a spacer 2220. In one embodiment, the spacer 2220 includes a distance regulator 2222 that includes a groove 2224, a fitting 2225, a junction 2226, a fork 2237 and a roller 2228. One end the junction 2226 is reversibly coupled to the groove 2224 via a fitting 2225. The fitting 2225 can be loosened to move the junction 2226 to a different position relative to the groove 2224 (e.g., to slide the junction 2226 up or down) and then the fitting 2225 can be tightened (e.g., via a knob or a set screw that tightens the fitting 2225 relative to the groove 2226). In this way, the distance regulator 2222 aids in control of the space (e.g., the distance) between the spacer 2220 and the waveguide 220. The groove 2224 and the fitting 2226 is merely one exemplary embodiment of a distance regulator 2222. Other suitable distance regulators 2222 substantially maintain the remaining portions of the spacer 2220 (i.e., the junction 2226, the fork 2227 and the roller 2228) at a controlled and/or substantially controlled distance from the waveguide 220 include, for example, an arch having stress and strain properties that enable it to maintain a controlled and/or a substantially controlled distance between the waveguide 220 and the remaining portions of the spacer 2220. Other distance regulators 2222 may have a shape substantially similar to a c-clamp. Other distance regulators 2222 could work via a substantially different mechanism such as, for example, a tube, a beam, or a formable locking conduit that can be shaped in a desired fashion and then locked in place by mechanical, chemical, electrical or other suitable means for the duration of the procedure. In one embodiment, a suitable distance regulator has a mechanism (e.g., a mechanical mechanism) that enables the distance of the spacer (e.g., the surface of the spacer that contacts the outside surface of the subject's body) relative to the waveguide to be located inside the subject's body to be dialed in and/or adjusted.

The location of the portion of the spacer that contacts the outside surface of the subject's body relative to the underlying waveguide tip is critical to the ability to regulate the distance at which the waveguide can penetrate into the subject's body. The location of the portion of the spacer that contacts the outside surface of the subject's body may be adjustable. For example, the location of the portion of the spacer that contacts the subject's body maybe adjusted to lead (e.g., be at least slightly ahead of), be adjacent to, and/or follow (e.g., be at least slightly behind) the waveguide tip. Optionally, the location of the portion of the spacer that contact the outside surface of the subject's body relative to the waveguide tip may be automatically adjusted to compensate for the area of treatment or may be adjustable by the practitioner in accordance with the practitioners medical judgment and/or preference. The use of the spacer in conjunction with an aiming beam can provide a visual guide of the tip location.

Use of the spacer enables the depth of the waveguide 220 to be substantially controlled. In this way, some of the “art” and/or “technique” and/or extensive training typically associated with aesthetic treatments inside a subject's body (e.g., traditional suction assisted liposuction) become less difficult to achieve. Controlling the waveguide depths to distances that avoid contour deformities and/or over treatment and/or under treatment of specific regions can make such treatments require less training and experience in order for a practitioner to get reliable and dependable results. In addition, with the availability of such a spacer providing control and/or feedback control (described later) the subject can expect less variability based on their choice of practitioner making such laser assisted procedures have a more predictable outcome.

In addition, referring also to FIGS. 34b and 34c, a fork 2227 and a roller 2228 are disposed on another end of the junction 2226. The roller 2228 couples to the fork 2228, by for example, a pin that connects to a first side of the fork 2227, through a portion of the roller 2228 (e.g., through the center of the roller 2228), and also connects to a second opposing side of the fork 2227. The fork 2227 may be employed to adjust the angle at which the roller 2228 contacts a skin surface. In this way, the fork 2227 angle and/or the diameter of the roller 2228 can also contribute to the distance between the spacer 2220 and the waveguide 220. The fork 2227 can be angled such that the distance between the waveguide 220 and the junction 2226 is larger than the distance between the outer diameter of the roller 2228 and the tip 225 of the waveguide 220.

The roller 2228 can be fabricated from, for example, a metal material such as aluminum. Alternatively, the roller 2228 can be fabricated from, for example, a material such as glass, quartz, sapphire, or a polymer (e.g., a transparent polymer, a colored polymer, or an opaque polymer). Use of a transparent material when fabricating the roller 2228 could allow the practitioner to see an aiming beam provided by the waveguide 220 through the roller to aid in guiding the practitioner's treatment. The external diameter of the roller 2228 contacts the outside surface of the subject's body during treatment. A roller 2228 is merely exemplary of suitable outside skin contact surfaces that may be employed as part of the spacer 2220. Other outside skin contact surfaces that may be employed as part of the spacer 2220 could have the shape of a skate, a ski, a ball, or any other shape suitable for movement on the external surface of the contact surface (i.e., the subject's skin) that results in a rolling and/or a sliding contact. In addition, the outside skin contact surface can be coated with a material (e.g., a lubricant) that facilitates its movement on the outside surface of the subject's body. Optionally, a fluid (e.g., a gas, a liquid, or a gel) may be spread onto the surface of the subject's body from the spacer 2220 for the purpose of reducing friction at the surface of the subject's body and/or providing skin cooling.

Thus, referring to FIGS. 34a, 34b, and 34c, one or more of the distance regulator 2222 (e.g., the position of the groove 2224 and the fitting 2225), the junction 2226, the fork 2227, and the roller 2228 can contribute to the constant or substantially constant distance between the spacer 2220 and the waveguide 220. Suitable distances between the outside skin contact surface of the spacer 2220 (e.g., the roller 2228, the skate, the ski, the ball, etc.) and the waveguide can be determined based upon the region being treated and the desired area for treatment (e.g., if it is desirable to coagulate the dermis as the hand piece 200 having the spacer 2220 is moved on the external surface and the waveguide 220 is moved on the internal surface of the subject's body). The targeted tissue treatment or treatments, e.g., the intended treatment use of each hand piece 200 may determine the suitable spacer 2220 distance. Suitable spacer distances range from about 10 mm to about 1 mm, from about 5 mm to about 7 mm, or from about 2 mm to about 3 mm. In an embodiment, where the practitioner wishes to treat deep fat, the spacer distance is adjusted to be about 5 mm. In an embodiment where the practitioner wishes to treat dermis and avoid organs such as muscle and bone, the spacer distance is adjusted to be about 2 mm.

A suitable junction 2226 can be made from any of a number of materials including, for example, polymer(s), copolymer(s), metal(s), and composite(s). In one embodiment, a junction 2226 is made from stainless steel. Suitable junctions 2226 may have any of a number of cross sectional shapes including, for example, round, rectangular, elliptical, or square. Where the cross sectional shape of the junction 2226 is round, the diameter ranges from about 1 mm to about 10 mm, or about 4 mm. The length of the junction 2226 ranges from about 2 inches to about 14 inches. In one embodiment, the junction 2226 has a stiffness that makes it easy to control. The fabrication material and/or the size (e.g., the diameter) of the junction 2226 can be selected to provide a desired level of junction 2226 deflection and/or a desired level of junction stiffness. For example, a deflection range may be employed that ranges from about 0.25 mm to about 4 mm, or about 2 mm of deflection for each pound of force.

The targeted tissue treatment or treatments, e.g., the use, of each hand piece 200 may also be determined or employed to determine the one or more selected wavelength emitted by the energy source through the hand piece 200. For example, one hand piece 200 may have multiple lasers capable of emitting different wavelength ranges while another hand piece 200 has a single laser capable of emitting a single wavelength range. In one embodiment, the waveguide 220 emits one or more wavelength ranges.

In one embodiment, the waveguide 220 emits a single wavelength range to, for example, treat skin tissue such as dermis tissue and/or treat fat tissue. In one embodiment, the waveguide 220 emits a multiple wavelength ranges to, for example, treat skin tissue such as dermis tissue and treat fat tissue either simultaneously or sequentially. When used simultaneously the multiple wavelength ranges can treat the skin in parallel to (e.g., simultaneous with) treatment of the fat.

The waveguide 220 can be fabricated from any of a number of materials including, but not limited to, the waveguide 220, the core 226 and the buffer 228 materials disclosed herein. The waveguide 220 can be self supporting, can be a bare fiber with or without a support structure such as described herein (e.g., in association with FIGS. 16-21). In addition, the waveguide 220 can be adjacent a cannula (e.g., the waveguide can be inside or external to a cannula). Any suitable cannula may be employed, for example, an optical cannula, a suction cannula, an ultrasound cannula, or a cannula typically employed in power assisted liposuction, for example. Suitable waveguide materials include, for example, quartz, optical glass, optical ceramics, garnet, and sapphire. The waveguide 220 can apply energy along the longitudinal axis or, alternatively, the waveguide 220 may be a side firing waveguide disclosed herein (see, e.g., FIGS. 8B-8G).

In one embodiment, the hand piece 200 has a spacer 2220 and a waveguide 220 that is side firing (a side firing waveguide is not shown in FIGS. 34A-34C, however is shown and disclosed in FIGS. 8B-8G). The side firing waveguide, in one embodiment, includes sapphire. In one embodiment, the waveguide 220 and the tip 225 are sapphire. In another embodiment, only the tip 225 is sapphire and the remaining portions of the waveguide 220 are made from a less costly material such as, for example, quartz, optical glass, or optical ceramic material.

One or more of the distance regulator 2222 mechanical configuration, the junction 2226 material and the waveguide 220 material combine to enable the distance between the outside skin contact surface (e.g., the roller 228) and the waveguide 220 to be maintained at a constant or substantially constant distance during treatment.

The side firing waveguide 220 may be employed together with the spacer 2220 to provide coagulation of skin tissue (e.g., dermis tissue) at a substantially controlled and substantially consistent depth determined by the spacer 2220 and the waveguide 220. In one embodiment, the side firing waveguide 220 achieves a larger area of coagulation than a waveguide that fires along its longitudinal axis when both the side firing and longitudinal firing waveguides are employed to treat dermis at the same depth, at the same power level, and at the same wavelength.

In one embodiment, 15 Watts of power is delivered and a side firing waveguide 220 applies a wavelength range including 1470 nm to a subject's dermis from inside the subject's body; the depth of the side firing waveguide 220 is substantially controlled by the spacer 2220 that together with the waveguide 220 controls the depth of the waveguide 220. Controlling the depth of the waveguide 220 within the subject's body can provide consistent treatment and can avoid treatment of unwanted tissues (e.g., organ(s), bone, etc.). The wavelength range applied to the dermis from inside the subject's body provide soft tissue coagulation that can provide tightening of the dermis and to an appearance of skin tightening on the external surface of the subject's skin. Such a procedure can be employed, for example, in the abdomen to tighten dermis such that the appearance of a tightened abdomen previously requiring surgical cutting and stitching can be achieved with relatively few entry points of the waveguide 220 into the abdominal wall. In this way, the downtime required for healing from the procedure and the risk of scarring associated with the procedure is diminished.

In order to target a desired tissue treatment or treatments, the one or more selected wavelength emitted by the energy source through the waveguide 220 can range from about 400 nm to about 11000 nm, from about 1900 nm to about 2700 nm, from about 400 nm to about 3000 nm, or about 400 nm to about 1200 nm, or about 400 to about 600. A power range of from about 1 Watt to about 100 Watts, or from about 10 Watts to about 50 Watts may be employed in continuous wave or in quasi continuous wave mode. In pulsed mode, a pulse width of from one femtosecond to about 10 milliseconds, or from about 10 microseconds to about 1 millisecond, or from one microsecond to continuous wave mode, or from about 1 millisecond to 5 seconds.

A fluence of from about 0.001 Joule/cm² to about 1000 Joule/cm², or from about 0.001 Joule/cm² to about 300 Joule/cm², or from about 5 Joule/cm² to about 150 Joule/cm², or from about 10 Joule/cm² to about 40 Joule/cm², 1410 to about 2100 and a fluence of from about 10 Joule/cm² to about 1000 Joule/cm², or from about 15 Joule/cm² to about 700 Joule/cm² may be employed. Referring still to FIGS. 34a-34c, disclosed herein is a mechanism to control a waveguide for treatment inside a subject's body and at least a portion of a spacer external to the subject's body, the waveguide inside the body is opposite the portion of the spacer in contact with the external surface of the subject's body. In one embodiment, a magnet is disposed in one of the portion of the spacer in contact with the external portion of the subject's body and the waveguide. In an embodiment where the magnet is disposed in the portion of the spacer in contact with the external portion of the subject's body, the magnet forces movement of the waveguide toward the spacer. The magnet acts to actively drives the tip toward the spacer thereby compressing the skin tissue and avoiding any tendency for the tip to separate from the spacer when traversing within a subject's body.

The outside skin contact surface (e.g., the roller 2228) may be employed to measure one or more of the velocity of hand piece 200 movement, the distance of the hand piece movement 200, and/or the time of hand piece 200 movement. One or more of these measurements can be relayed to or fed back to the laser. Such measurements enable the laser to create a treatment pattern on the subject's tissue, the treatment pattern informed by the relayed measurements. For example, the laser pulse can be altered in light of the feedback from the outside skin contact surface of the spacer 2220 (e.g., the roller 2228) to enable a pulsed laser to create a consistent or substantially consistent treatment pattern in the subject's tissue.

The outside skin contact surface of the spacer 2220 (e.g., the roller 2228) can employ any of a variety of sensing mechanisms. Suitable sensing mechanisms include, for example, optical, electrical, thermal, ultrasound, magnetic, and/or IR, for example. The outside skin contact surface of the spacer 2220 provides a fixed reference relative to the waveguide 220 (e.g., the waveguide tip 225). One can make use of the fixed distance and/or reference of the outside skin contact surface relative to the tip 225 of the waveguide 220 and make use of this data point for feedback. Because the outside skin contact surface is a fixed reference relative to the waveguide 220 tip 225 employing a sensing device in or adjacent the outside skin contact surface can provide control of treatment that improves safety. For example, if the waveguide 220 tip 225 drifts feedback control provided by the sensor can enable adjustment (e.g., adjustment to the handle 210 of the hand piece 200, and/or adjustment to the tension of the distance regulator 2222) to correct the drift. More specifically, the feedback control can enable mechanical adjustment of a portion of the hand piece 200 and/or adjustment by the practitioner to correct the drift. In another embodiment, an ultrasound sensor can determine the distance of the waveguide 220 from the sensor thereby determining and/or confirming the distance of the spacer. In another embodiment, sensing mechanism(s) passively monitor the relationship between the outside skin contact surface and the waveguide 220 to ensure that the distance falls within desirable limits. Optionally, when the distance falls outside the desirable limits the power is switched off requiring intervention by the practitioner to adjust the distance to within the desired limits. In another embodiment, the sensing mechanism(s) actively monitor the relationship between the outside skin contact surface and the waveguide 220 and adjust at least one of the spacer 2220 and the waveguide 220 to ensure that the desired distance is substantially maintained (e.g., the sensor feedback signals the required adjustment to the device to correct the distance). The sensor can measure the drag force that is required by the practitioner to traverse the tissue being treated. The sensor can be passive providing a feedback signal regarding the required force to traverse the target tissue (e.g., a sound or visually available measurement or other signal) to the practitioner. The sensor can be active adjusting (e.g., increasing) the power supplied to the waveguide 220 when the drag force hits a certain level, the change in power level enables the practitioner to traverse the target tissue with the level of power suited to the draft presented by the target tissue. For example, where a relatively high level of drag is encountered the feedback signals for an increase in power level that enables the practitioner to traverse the target tissue with less drag due to the increase in power level.

In one embodiment, the outside skin contact surface of the spacer 2220 (e.g., the roller 2228) includes a velocity sensor that ensures that the there is the correct amount of energy being delivered to the waveguide 220 per distance being traversed that is needed to achieve the desired treatment of the tissue. The sensor can provide feedback that enables certain treatment parameters to be tailored to the treatment in real time. For example, the sensor can provide feedback that enables a suction force to be tailored to the liberated and/or melted fat in the region of treatment. The sensor can provide feedback informing that the waveguide is adjacent to tissue that is undesirable to be treated (e.g., bone or organs) and accordingly the power level is changed such that the discharge rate is dropped for safety reasons.

In one embodiment, the outside skin contact surface of the spacer 2220 (e.g., the roller 2228) includes a drive mechanism that can be active and/or passive. The drive mechanism can pull the outside skin contact surface of the spacer 2220 (e.g., the roller 2228) forward at a desirable rate (e.g., a desirable speed).

Such sensors employed with the spacer 2220 (e.g., the roller 2228) increase safety. This is important with active treatment with energy sources when we want to be sure to deposit energy at the right depth. We can control the results of the treatment by measuring the light transmission to the skin and/or measuring the temperature of the skin so that we can avoid burning of skin, vascular, nerve and other organs. The outside skin contact surface of the spacer 2220 (e.g., the roller 2228) can be, for example, transparent to enable visualization of the waveguide treatment through the contact surface of the spacer 2220 via, for example, the aiming beam. This outside skin contact surface of the spacer 2220 (e.g., the roller 2228 or a glider) can provide cooling energy, a magnetic force, acoustical signals, acoustical energy, light energy, radio frequency energy to enable you to provide more uniform treatment, to provide supplemental treatment or modify the treatment so that it adds to and/or complements the treatment with the waveguide 220.

Alternatively, the outside skin contact surface (e.g., the roller 2228) may incorporate all or a portion of sensor that enables determination of the position of the roller in space. For example, a tachometer may placed in the roller 2228 to measure the speed of the roller 2228 and thereby to enable a consistent treatment pattern to be applied even in an instance of a changing speed of treatment.

Referring still to FIGS. 34a-34c, in accordance with treating a subject's dermis to accomplish skin tightening using the hand piece 200 having a spacer 2220, all personnel in the treatment room should wear the appropriate laser protection eyewear. The areas to be treated are cleaned with antibacterial solution. If necessary, a sedative may be given to the subject either orally or by IM injection. Using a sterile surgical marking pen, the practitioner marks the treatment locations. Local anesthesia appropriate to the procedure can be applied, for example, tumescent anesthesia may be used.

The practitioner creates a 1 mm incision in the subject's body through which the waveguide 220 can be inserted. The practitioner positions the end 224 of the waveguide 220 at the intended area of treatment. The outside skin contact surface is the roller 2228, which contacts the outside surface of the subject's skin. The practitioner desires to treat the skin from inside the subject's body. It is desirable that the waveguide be positioned inside the subject's body under the skin at a distance from the roller 2228 suitable to treatment of the dermis without perforating the skin. The distance between the outside contact surface, e.g., the roller 2228, and the waveguide 220 is about 2 mm. The waveguide 220 is a side firing waveguide (described in conjunction with FIGS. 8B-8G), which is suited for the treatment of dermis tissue. During treatment micro damage patterns of coagulated and/or ablated dermis and/or epidermis are created in the skin and/or in the hypodermis. Where the tissue is coagulated a wavelength range including a wavelength of about 1.44 micron or about 1.47 micron is employed, where the tissue is ablated a wavelength range including a wavelength of about 2.94 micron is employed. The laser is at a power level of about 20 watts. Where the waveguide 220 is fired in pulsed mode a pulse width of from about 10 ms to about 1 second is employed. When the hand piece 200 is moved across the subject's skin the speed of movement may be controlled using a speed sensor disposed in the roller 2228. Suitable speed sensors can be optical or mechanical (e.g., like wheels optionally employed together with a tachometer) for example. The pulse repetition rate can be delivered as a function of the speed measured by the sensor. Employing such a sensor together with the waveguide can enable a substantially controlled and/or uniform treatment that enables damage patterns to be distributed substantially uniformly. For example, the multiple adjacent damage zones are formed by the treatment with the hand piece 200. The use of such a speed feedback sensor that works in conjunction with the delivery of energy by the waveguide 220 can enable the practitioner to move at any speed and still keep the damage pattern substantially consistent. Using the hand piece 200, the practitioner moves the waveguide 220 such that it traverses inside the subject's body under the dermis tissue to be treated. The practitioner can move the hand piece 200 in any of a number of ways, for example, in a forward only motion, forward and back, in a fan like motion, and/or in a grid like pattern, forward along a first longitudinal axis to create a first treatment line and backward adjacent the first line to create a second treatment line.

The distance between damage zones when such speed sensor feedback is provided in the pulse mode may be measured by:

Z=T*v

where
Z is the distance between damage zones,
T is the period between pulses e.g., the pulse width, and
v is the speed of the device.

Where is desired to have a constant distance Z between damage zones it is desirable that the period between pulses T be constant, accordingly, the control system removes time as a factor such that a consistent distance Z between the pulses (and areas of damage) is always achieved.

Alternatively, during the above described dermis treatment procedure, the side firing waveguide 220 may be fired in continuous wave (CW) mode at a power level of about 20 watts. Employing a CW mode enables maintenance of uniform distance parameters (e.g., like lines). Thus, in order to control damage the CW power is regulated relative to the speed implemented by the practitioner. If such power control were not provided the amount of energy provided to the treatment area would vary in accordance with the speed of treatment such that in areas of relatively higher speed of waveguide 220 movement there would be relatively less energy provided to the tissue and in areas of relatively lower speed of waveguide movement there would be relatively more energy provided to the tissue. Thus, without the power control employed in conjunction with the CW treatment inconsistent treatment (e.g., under treatment and/or over treatment such as burns are possible).

Where power control is employed, a relatively consistent level of treatment of the tissue may be achieved such that a relatively consistent amount of energy is delivered to create a line of damage. The line of damage is at a substantially consistent depth determined by the spacer 2220 and the waveguide 220.

Optionally, the waveguide 220 has an aiming beam aimed from inside the patients body under the skin such that the practitioner can see the aiming beam, which indicates and provides feedback regarding the region of treatment. For example, the roller 2228, can be transparent to enable the aiming beam to shine there through during the treatment of the dermis by the practitioner.

In another embodiment, a waveguide has a distal end having a hemisphere shape. Optionally, an end of the waveguide is formed into the shape of a hemisphere. In another embodiment, an optical sphere is integrated into the waveguide thereby to provide an end having a hemisphere shape.

In another embodiment, all or a portion of a waveguide is “frosted” e.g., designed such that the “frosted” portions of the waveguide provide a relatively even distribution of energy, have a relatively low energy density, and/or scatter energy. A whole waveguide, a section of the waveguide, or multiple sections of the waveguide (e.g., stripes or spots) may be “frosted.” All or a portion of the waveguide can be “frosted” by utilizing a glass fiber containing bubbles the provides diffusion relative to a non bubbled quartz or sapphire fiber. Alternatively, all or a portion of the waveguide can be etched (e.g., sandblasted) or covered with a coating (e.g., ceramic coating by deposition) to promote diffusion and/or scattering of the electromagnetic energy transmitted through the waveguide. In this way, energy density is reduced in the treated (e.g., the “frosted”) regions. For example, where the entire waveguide is “frosted” via etching to provide a relatively even distribution of energy allows the electromagnetic energy to be broadly directed. In one embodiment, due to the breadth of electromagnetic radiation direction, the waveguide can be placed in an area to be treated and due to the lessened energy density and scattering properties the waveguide does not require constant (or optionally any) movement when treating the soft tissue. When a waveguide is treated by etching, sandblasting, or by providing rough covering (e.g., ceramic) a smooth cover can be employed to protect tissue that may come in physical contact with the waveguide surface.

It may be desirable for a portion of the waveguide, for example the tip, to be heated to enable the tip to lead the waveguide in traversing through tissue. In one embodiment, the absorption by a waveguide tip of a portion of the emitted light is controlled to provide controlled heating of the waveguide tip. For example, an absorbtive coating such as, for example, chromium oxide, iron oxide, and/or carbon coating deposition can be disposed on all or a portion of the waveguide and/or on the waveguide tip. In another embodiment, the tip of the waveguide is made of an absorbtive or a partially absorbtive material such as, for example, a filter glass or a colored glass. A surface treatment can be applied to the tip so that the tip is enabled to hold particles from its emission of electromagnetic energy to be heated. Suitable surface treatments include, for example, providing roughness via etching or deposition of materials. In order to control the heating of the tip the quantity and size and location of the roughness may be controlled.

In accordance with the devices and methods disclosed herein, feedback information and/or feedback control can be employed. Suitable methods for feedback control include, for example, measuring the infrared radiation of the waveguide tip, measuring the infrared radiation of the treated tissue (e.g., the melted fat tissue), and/or measuring the temperature of the external surface of the subject's body in the region of the treatment with the waveguide.

In another embodiment, irradiation is controlled as a function of the derivative of, the integral of, or the power spectrum of the thermal radiation detected or any combination thereof. In another embodiment, irradiation is controlled as a function of specific frequency components or one or more range of specific frequency components of the power spectrum of the thermal radiation detected. Finally, the irradiation can be controlled as a function of power. In another embodiment, irradiation is controlled as a function of the intensity and/or power of infrared radiation.

One or more of the wavelength and/or power level may be altered in response to the measured infrared radiation. In this way, over treatment and/or under treatment due to temperature may be substantially avoided. In one embodiment, the back scattering of the delivered energy (e.g., the backscattering of the optical radiation) is measured.

In one embodiment, during tissue treatment the treatment alters optical properties of the tissue. Accordingly, the backscattering measured at the treated tissue could change. Tracking this change as a function of time enables feedback of the treatment.

In another embodiment, the movement (i.e., the speed) of the hand piece and/or waveguide and/or at least a portion of a spacer that is connected to the hand piece and external to the subject's body are measured. The information about the movement can enable feedback control to alter the power level of the waveguide in response to the movement, the absence of movement, and/or the speed. By altering the power level in response to changes in movement, over treatment of an area by exposing it to too much energy and/or under treatment of an area by exposing it to too little energy can be avoided. Accordingly, a substantially consistent treatment of a region of tissue can be achieved.

In another embodiment, acoustic feedback may be utilized by employing one or more microphone in at least a portion of the hand piece (e.g., in the waveguide and/or a portion of a spacer). During treatment a phase change of the tissue is expected. For example, the tissue may bubble, sizzle, vaporize and there are acoustic signals associated with phase transition such as, vaporization, bubbling, sizzling etc. Employing the microphone to measure specific frequencies associated with the phase transition acoustic signal enables feedback control.

Employing feedback control can protect the subject from over treatment. In addition, the use of feedback control can avoid failure of the hand piece or of a portion of the hand piece such as the waveguide. Avoiding failure of the waveguide can include avoiding a portion of the waveguide from becoming misshapen (e.g., avoiding a change in waveguide tip shape) and/or avoiding a loss of integrity of at least a portion of the waveguide from becoming, for example, brittle and/or cracked. In one embodiment, in order to avoid uncontrolled temperature when the hand piece, a portion of the hand piece such as the waveguide, the waveguide tip, the temperature of the treated tissue (e.g., the melted fat tissue), and/or the external surface of the subject's body in the region of the treatment measures at and/or higher than a threshold temperature than via feedback control the system shuts down and/or reduces the power level of the system in order to avoid overheating and the resulting risks to the subject's tissue and/or risks to the device.

Employing feedback control in accordance with the disclosed methods and/or devices enables a higher power level to be employed with less risk to the subject and/or the device (e.g., the hand piece and/or the waveguide) than in methods and/or devices that lack feedback control. For example, in one embodiment, a waveguide was set at a 40 watt setting and to 0.7 kJ, and the waveguide exposed to these conditions fractured (e.g., became brittle and cracked) at the tip. This failure occurred after about 18 seconds of firing/treatment. In another embodiment, the waveguide was set at a 20 watt setting and to 2 kJ and after firing for about 100 seconds the waveguide did not show any damage to the waveguide surface and/or the waveguide tip.

In summary, referring again to FIGS. 1-4, the base unit 110 and/or the hand piece 200 provide EMR that is preferentially absorbed by a specific chromophore in the treated tissue that in turn heats the surrounding tissue and/or liquefy fat. The time of laser action is dependent on factors including the amount of fat, tissue resistance, and extension of treatment area. The hand piece 200 suitable for laser assisted lipolysis can deliver predetermined wavelengths to the treatment area.

In one embodiment, the System 100 operates according to the parameters provided in Table 1. The optical energy wavelengths are produced within a diode bundle and include 920 nm, 970 nm, 1064 nm or 1320 nm and each of the diodes has a +/−10 nm potential for wavelength variation. The base unit 110 and/or the hand piece 200 enable energy delivery using an optical fiber 230. Energy is delivered in the near infrared, which ranges from 910 nm to 1320 nm. The base unit 110 and/or the hand piece can operate in pulsed mode and is capable of operating in continuous wave (CW). The base unit 110 and/or the hand piece 200 parameters and other system 100 features are controlled using the user interface panel 120. The user interface panel 120 allows the user to program pulse duration and average power. In one embodiment, the user of the user interface panel 120 is also the practitioner. Alternatively, the user of the user interface panel 120 is another individual. In some embodiments, the user interface panel employs software that executes according to the user's instructions to the user interface panel.

TABLE 1
System Parameters

| | System Parameters |
|---|---|
| Light Source/Laser Type | Diode laser |
| Wavelengths (User Selectable) | 920, 970, 1064 and 1320 nm |
| Bandwidth Variation | +/−10 nm |
| Operation Mode | Pulsed & CW |
| Repetition Rate | Up to 100 Hz, CW |
| Frequency: | Up to 100 Hz |
| Pulse Width | 100 ms - CW |
| Pulse Duration | 100 ms-continuous wave (CW) |
| Average Power | 5-80 W |
| Method of Optical Output | Optical fiber (200 to 1500 μm) |
| Fiber Diameter | 200-1500 μm |
| Aiming beam | 1-10 mW @ 635 nm diode |
| Cooling | 10 to 30° C. |
| Cooling Method | Self-contained, internal cooler |
| Actuator | Footswitch trigger |
| Electrical Specifications | 115/230 VAC/15A, 50/60 Hz, |
| | single phase |
| Operating Environment | Temperature: 15-28° C. |
| | (55-80° F.) |
| | Humidity: 0-40% |

Referring now to FIG. 1, optionally, the hand piece 200 suitable for laser assisted lypolysis is packaged as a sterile one-time use attachment. In one embodiment, prior to packaging, the hand piece 200 is sterilized using gamma radiation. In some embodiments, the base unit 110 and the cluster connector 130 are individual non-sterilized finished components. In one embodiment, the laser is a direct diode laser delivery system. The practitioner selects the desired wavelength. Laser pulses are generated at user-selectable exposure times from about 100 ms to continuous (CW) operation. The average power of the diode laser is adjustable from about 5 watts to about 80 watts, from about 5 watts to about 30 watts, from about 40 watts to about 60 watts, or about 24 watts. A coolant circulation system directly cools the diode(s) to prevent overheating. A pump circulates coolant that directly cools a heat sink attached to the diode. The coolant circulates as long as the base unit is turned on. A High Voltage Power Supply transforms 115 VAC to a high voltage DC that supplies electrical energy to the diode(s) that activate and sustain the lasing action. A Laser Control Module is the electronic circuitry that controls the following functions: the front control panels, laser energy calibration, and the safety interlock system. The foot switch is an electrical switch which actuates treatment of the treatment area via the hand piece 200.

Referring to FIGS. 1 and 4, the hand piece 200 optical fiber 230 connects to the base unit 110. Via the optical fiber 230, the hand piece delivers laser light to the waveguide 220 which is inserted in the subject's body. In one embodiment, an aiming beam follows the same path as the laser light, through the optical fiber 230, the waveguide 220, and into the subject's body. The aiming beam enables determination of the treatment location and the integrity of the waveguide 220. If the aiming beam is not visible at the tip 225 of the waveguide 220, if its intensity is reduced, or if it looks diffused, this is a possible indication of a damaged or not properly working base unit 110 or hand piece 200.

In accordance with treating a subject via laser assisted lypolysis using the hand piece 200 described herein, all personnel in the treatment room should wear the appropriate laser protection eyewear. The hand piece 200 is connected to the cluster connector 130. The subject's weight is recorded. The areas to be treated are cleaned with antibacterial solution. If necessary, a sedative may be given to the subject either orally or by IM injection. The practitioner evaluates and estimates the volume of fat in the treatment area and the amount to be removed. Using a sterile surgical marking pen, the practitioner marks the treatment locations.

Local anesthesia appropriate to the procedure is applied, for example, tumescent anesthesia may be used, but need not be used in alternate embodiments. The volume of tumescence solution used is based on the volume of fat to be removed with the maximum volume of 1 liter.

The practitioner creates a 1 mm incision through which the waveguide 220 can be inserted. The practitioner positions the end 224 of the waveguide 220 at the intended area of treatment. The aiming beam can be seen under the skin to indicate the region of treatment, for example, in one embodiment the aiming beam indicates the center of the treatment spot.

The waveguide 220 should not be placed deeper than fat tissue. If the practitioner feels a significant increase in resistance in the advancement of the waveguide 220 or observes a sudden decrease in the visibility of the aiming beam through the skin, this is an indication that the waveguide 220 has been placed too deep into the tissue. The practitioner should reposition the end 224 of the waveguide 220 so that it is less deep and within the fat tissue before delivering energy to the tissue. The practitioner selects the desired wavelength.

The practitioner presses the footswitch to deliver laser energy to the treatment site. Using the hand piece 200, the practitioner moves the waveguide 220 back and forth in a fan-like motion over the area to be treated, moving the end 224 of the waveguide 220 to various depths though not deeper than the fat tissue. The laser energy will melt the fat. The melted fat can be aspirated by, for example, a syringe or aspiration cannula. As a general guideline, between 60 to 80 joules of energy will lipolyze one gram of fat. The practitioner palpates the area to ensure symmetrical removal of fat. The practitioner continues reciprocating (moving the waveguide 220 back and forth in a substantially fan like motion and/or in a substantially longitudinal motion) until there is sufficient disruption of the fatty tissue. Once a sufficient volume of fat has been treated, lasing is discontinued. The laser is secured and the waveguide 220 is withdrawn from the treatment site.

The waveguide 220 may be maintained in the sterile field for further lasing if desired. To aspirate the liquefied fat, an aspiration syringe may be inserted into the treatment site to remove the melted fat from the body. For ease of liquid fat removal, large diameter aspirating cannulas may be used in place of an aspiration syringe, for example. If using an aspirating cannula an accompanying peristaltic pump may be used. If additional fat is to be removed, the waveguide 220 is reintroduced into the treatment site and the earlier described process is repeated.

Time of laser action is dependent on the amount of fat, tissue resistance, and extension of treatment area. The passage of the waveguide 220 produces tracts in the fat, liberating an oily substance from the adipocytes (e.g., lipid liberation). Once sufficient fat has been removed and symmetry has been achieved, the clinical endpoint has been reached. The practitioner cleans the wound with an antiseptic solution, and then closes the wound with an adhesive skin (e.g., steri-strips or sutures) closure if needed. Following the procedure, the practitioner uses standard post-op pressure dressing including compression garments. Post-op wound care instructions should be given and explained to the subject.

Referring to FIGS. 1, 2, 4, and 5A in another embodiment of a method of treatment, the practitioner creates a 1 mm incision through which the waveguide 220 can be inserted. The practitioner positions the end 224 of the waveguide 220 at the intended area of treatment, the dermal-hypodermal junction. The aiming beam can be seen under the skin to indicate the region of treatment. The practitioner treats adipose tissue in the dermal-hypodermal junction with a wavelength range that includes 924 nm, which targets lipid rich tissue to melt adipose tissue (e.g., liberate lipids in the adipose tissue). The practitioner selects a wavelength range that includes 975 nm, which targets dermal tissue to heat and/or coagulate dermal tissue to provide a skin tightening effect. Optionally, the system 100 includes a governor that limits the amount of power delivery at the wavelengths being administered. For example, the system 100 can include a governor that enables of a percentage of power (e.g., wattage) in the system at a certain wavelength range according to the following table:

| | Wattage at a | Wattage at a |
|---|---|---|
| | wavelength range | wavelength range |
| Setting | including 924 nm | including 975 nm |
| 1 | 0% | 100% |
| 2 | 33% | 67% |
| 3 | 50% | 50% |
| 4 | 67% | 33% |
| 5 | 100% | 0% |

Employing a power delivery governor in accordance with a system 100 enables the user to select a pre set power delivery range prior to beginning the procedure. Thus, in an instance where it is desirable to treat only skin, setting 1 may be selected, which directs all of the power delivery to the wavelength range that targets water and is effective for treating skin tissue. In an instance where it is desirable to equally treat skin tissue and adipose tissue, setting 3 may be selected, which delivers substantially equal levels of power to the wavelength ranges that target treatment of both adipose tissue and skin tissue skin. Where areas of thick skin are being treated together with adipose tissue, setting 2 might be selected to drive additional power to the thicker skin through the wavelength that includes 975 nm while also driving a lesser amount of power to the adipose tissue. Where areas of thin skin are being treated together with adipose tissue, setting 4 might be selected to drive relatively less power to the thinner skin through the wavelength that includes 975 nm while driving a relatively larger amount of power to the adipose tissue.

What is claimed is:

1. A method for treatment of tissue with a device, comprising:
inserting a treatment device into a soft tissue on an inside surface of a subject's body;
irradiating the soft tissue with a first wavelength range of electromagnetic radiation; and
irradiating the soft tissue with a second wavelength range of electromagnetic radiation.

2. The method of claim 1 further comprising:
generating electromagnetic radiation.

3. The method of claim 1 further comprising:
initiation of increased absorption of at least one wavelength at or near an end of the treatment device prior to insertion.

4. The method of claim 1 further comprising:
measuring the infrared radiation at or near an end of the treatment device or measuring infrared radiation at or near the irradiated soft tissue; and
controlling irradiation as a function of the measured infrared radiation.

5. The method of claim 1 wherein the treatment device comprises a self supporting waveguide.

6. The method of claim 1 wherein the treatment device comprises a waveguide forming a tube and adapted to evacuate at least a portion of irradiated tissue through the tube.

7. The method of claim 6 wherein the treatment device comprises an aperture adapted to evacuate at least a portion of irradiated tissue.

8. The method of claim 1 wherein the treatment device comprises a side firing waveguide.

9. The method of claim 1 wherein the first wavelength range is suitable for liberating lipid from adipose tissue and the second wavelength range is suitable for treating at least one of proteins, fibrotic components of adipose tissue, and skin tissue.

10. The method of claim 1 wherein the first wavelength range is suitable for melting adipose tissue and the second wavelength range is suitable for coagulating skin tissue or ablating skin tissue.

11. The method of claim 1 wherein the first wavelength range is suitable for melting adipose tissue and the second wavelength range is suitable for ablating one or more holes in skin tissue.

12. The method of claim 11 further comprising:
removing at least a portion of the melted adipose tissue through the one or more holes in skin tissue.

13. The method of claim 11 further comprising:
filling the one or more holes in skin tissue with adipose tissue.

14. The method of claim 11 further comprising:
applying suction to the one or more holes to assist adipose tissue removal through the one or more holes in skin tissue.

15. The method of claim 1 further comprising:
removing at least a portion of a pigmented lesion, at least a portion of a vascular lesion, at least a portion of a wrinkle, or at least a portion of a tattoo with a wavelength range of electromagnetic radiation suitable for ablating soft tissue or a wavelength range of electromagnetic radiation suitable for coagulating soft tissue.

16. The method of claim 1 wherein the second wavelength range is suitable for coagulating skin tissue.

17. The method of claim 1 wherein at least one of the first wavelength range and the second wavelength range tightens skin tissue.

18. The method of claim 1 wherein at least one of the first wavelength range and the second wavelength range tightens adipose tissue.

19. The method of claim 1 further comprising:
a third wavelength range suitable for coagulation of blood vessels.

20. The method of claim 1 further comprising:
suctioning melted adipose tissue with an aspiration device.

21. The method of claim 1 wherein the first wavelength range is generated prior to the second wavelength range.

22. A method for treatment of tissue with a device, comprising:
inserting a waveguide into a soft tissue adjacent on an inside surface of a subject's body, wherein the waveguide is self supporting; and
irradiating the soft tissue with at least one wavelength range of electromagnetic radiation.

23. The method of claim 22 further comprising:
generating electromagnetic radiation.

24. The method of claim 22 wherein the waveguide is capable of delivering power with multiple wavelength ranges.

25. The method of claim 24 wherein a first wavelength range is suitable for liberating lipid from adipose tissue and a second wavelength range is suitable for treating at least one of proteins, fibrotic components of adipose tissue, and skin tissue.

26. The method of claim 24 wherein a first optical radiation wavelength is generated prior to a second optical radiation wavelength.

27. The method of claim 22 further comprising:
suctioning melted adipose tissue with an aspiration device.

28. The method of claim 29 further comprising:
moving the waveguide within the soft tissue.

29. A device for the treatment of tissue inside a subject's body:
a source of electromagnetic radiation;
a self supporting waveguide having a distal end configured to deliver electromagnetic radiation and a proximal end configured to receive electromagnetic radiation; and
an interface for connecting the waveguide to the source of electromagnetic radiation.

30. The device of claim 29 wherein at least a portion of the waveguide has had an initiation of increased absorption of at least one wavelength.

31. The device of claim 29 further comprising:
a means to measure the infrared radiation at or near an end of the treatment device or a means to measure infrared radiation at or near the irradiated soft tissue; and
a means to control irradiation as a function of the measured infrared radiation.

32. The device of claim 29 wherein the waveguide is adapted to side fire.

33. The device of claim 29 wherein one portion of the waveguide is made from a first material and another portion of the waveguide is made from a second material.

34. The device of claim 33 wherein the first portion of the waveguide is made from transparent material with refractive index of at least 1.51.

35. The device of claim 33 wherein the first portion of the waveguide is made from sapphire.

36. The device of claim 29 wherein the waveguide substantially side fires in one type of tissue and the waveguide fires substantially along the longitudinal axis in another type of tissue.

37. The device of claim 29 wherein the waveguide is hollow.

38. The device of claim 29 wherein the waveguide is a tube.

39. The device of claim 38 wherein the tube comprises a transparent material.

40. The device of claim 38 further comprising a suction means that pulls tissue from a distal end of the tube to a proximal end of the tube.

41. The device of claim 38 wherein the tube leaks electromagnetic radiation along at least a portion of the inside surface of the tube

42. The device of claim 29 wherein a portion of the distal end is shaped.

43. The device of claim 29 wherein a portion of the distal end is bullet shaped.

44. The device of claim 29 wherein the proximal end is disposed in one side of an adaptor, the proximal end of the waveguide is adjacent to a distal end of a fiber disposed in the other side of the adaptor.

45. The device of claim 29 wherein the proximal end is disposed in one side of an adaptor, the proximal end of the waveguide contacts a distal end of a fiber disposed in the other side of the adaptor.

46. The device of claim 44 wherein the adaptor mechanically aligns or optically aligns the waveguide and the fiber.

47. The device of claim 44 wherein the adaptor is disposed within a hand piece.

48. The device of claim 29 wherein an end of the fiber couples to a base unit housing with source of electromagnetic radiation.

49. The device of claim 29 wherein the interface is a first connector adapted to connect to a second connector disposed on a base unit housing a source of electromagnetic radiation.

50. The device of claim 29 wherein the first connector comprises a locking mechanism adapted to prevent waveguide replacement or the first connector comprises a locking mechanism adapted to prevent coupling the first connector multiple times.

51. The device of claim 29 the device further comprising a control tag adapted to limit the length of time the waveguide may be used or adapted to control actuation of the source of electromagnetic radiation.

52. The device of claim 29 further comprising a sheath sized to surround at least a portion of the waveguide, the sheath adapted to contain at least a portion of the waveguide when the waveguide breaks.

53. The device of claim 29 wherein the distal end is configured to deliver multiple wavelengths of electromagnetic radiation.

54. The device of claim 29 wherein the distal end is configured to deliver a first optical radiation wavelength range suitable for melting, coagulating or inducing apoptosis of adipose tissue and a second optical radiation wavelength range suitable for treating skin tissue.

55. The device of claim 29 further comprising a tube adjacent the waveguide.

56. The device of claim 29 wherein the waveguide is external the tube.

57. A device for the treatment of tissue inside a subject's body:
a source of electromagnetic energy;
a waveguide having a distal end configured to deliver electromagnetic radiation in a selective direction and a proximal end configured to receive electromagnetic radiation; and
an interface for connecting the waveguide to a source of electromagnetic radiation.

58. The device of claim 57 wherein the distal end is angled thereby to enable the waveguide to deliver a substantial portion of the electromagnetic radiation at an angle of greater than about 10 degrees relative to the waveguide longitudinal axis.

59. The device of claim 57 wherein one portion of the waveguide is made from a first material and another portion of the waveguide is made from a second material.

60. The device of claim 59 wherein the first portion of the waveguide is made from a material with refractive index of at least 1.51

61. The device of claim 59 wherein the first portion of the waveguide is made from a sapphire material.

62. The device of claim 57 wherein the waveguide substantially side fires in one type of tissue and the waveguide fires substantially along the longitudinal axis in another type of tissue.

63. A method for treatment of tissue with a device, comprising:
inserting a waveguide into an inside surface of a subject's body, wherein the waveguide has a distal end configured to deliver electromagnetic radiation in a selective direction; and
irradiating a soft tissue with at least one wavelength range of electromagnetic radiation.

64. The method of claim 63 further comprising generating electromagnetic radiation.

65. The method of claim 63 wherein the distal end substantially side fires in one type of tissue and the distal end fires substantially along the longitudinal axis in another type of tissue.

66. The method of claim 63 wherein an angled distal end delivers a substantial portion of the electromagnetic radiation at an angle of greater than 10 degrees relative to the waveguide longitudinal axis.

67. The method of claim 63 wherein the at least one wavelength range is suitable for coagulating soft tissue.

68. The method of claim 67 wherein the wavelength range releases tension in at least a portion of the soft tissue.

69. The method of claim 63 wherein the at least one wavelength range is suitable for ablating soft tissue.

70. The method of claim 69 wherein the wavelength range cuts the soft tissue.

71. The method of claim 63 wherein the soft tissue is septa tissue.

72. The method of claim 63 wherein the soft tissue is skin tissue.

73. The method of claim 63 wherein the soft tissue is fibrous septa.

74. The method of claim 63 further comprising:
aligning the soft tissue with the waveguide by an aiming beam.

75. The method of claim 63 further comprising:
applying an aiming beam inside the subject's body for treatment feedback control.
